(19) United States
(12) Patent Application Publication
(10) Pub. No.: US 2026/0078452 A1
(43) Pub. Date: Mar. 19, 2026

(54) METHODS OF CANCER DETECTION USING EXTRAEMBRYONICALLY METHYLATED CPG ISLANDS

(71) Applicants: President and Fellows of Harvard College, Cambridge, MA (US); Dana-Farber Cancer Institute, Inc., Boston, MA (US)

(72) Inventors: Jiantao Shi, Jamaica Plain, MA (US); Zachary D. Smith, Cambridge, MA (US); Alexander Meissner, Cambridge, MA (US); Franziska Michor, Cambridge, MA (US)

(21) Appl. No.: 19/397,424

(22) Filed: Nov. 21, 2025

Related U.S. Application Data
(63) Continuation of application No. 18/268,240.
(60) Provisional application No. 63/126,863, filed on Dec. 17, 2020.
(60) Provisional application No. 63/246,306, filed on Sep. 20, 2021.

(51) Int. Cl. C12Q 1/6886; G16B 20/10; G16B 40/20

(52) U.S. Cl. CPC C12Q 1/6886; G16B 20/10; G16B 40/20; C12Q 2600/154

(57) ABSTRACT

The present invention relates to methods of characterizing cell-free DNA (cfDNA), detecting cancer, detecting the eradication of cancer, and determining a probability distribution of haplotypes. The methods use the data from genomic sequences from CpG Islands (CGI) methylated in the genome of extraembryonic ectoderm (ExE) to determine a proportion of fully methylated haplotypes in order to characterize the cfDNA sample and detect certain cancers.

### RELATED APPLICATION(S)

This application is a continuation application of U.S. application Ser. No. 18/268,240, filed on Jun. 18, 2023, which is a national stage filing under 35 U.S.C. 371 of International Application PCT/US2021/064210, filed Dec. 17, 2021, which claims priority to U.S. Provisional Application No. 63/126,863, filed on Dec. 17, 2020, and U.S. Provisional Application No. 63/246,306, filed on Sep. 20, 2021. The entire teachings of the above applications are incorporated herein by reference. International Application PCT/US2021/064210 was published under PCT Article 21(2) in English.

### GOVERNMENT SUPPORT

This invention was made with government support under GM099117 awarded by the National Institutes of Health (NIH). The government has certain rights in the invention.

### BACKGROUND OF THE INVENTION

The overwhelming majority of cancer-related deaths result from complications of metastatic disease. Modern anti-cancer therapies generally fail on metastatic disease due to tumor evolution [1], allowing heterogeneous cancer cell populations to acquire novel traits that enable them to escape from therapies, colonize new sites, and become more aggressive over time. Early diagnosis of disease leads to much-improved prognosis compared to advanced stage disease and can be based on imaging- or blood-based testing [2]. Although serum-based protein biomarkers such as carcinoma antigen-125 (CA-125) [3], carcinoembryonic antigen (CEA) [4], and prostate-specific antigen (PSA) [5] have been used to track the progression of specific cancer types, they lack the sensitivity and specificity necessary for detection of early stage diseases.

Liquid biopsies based on the analysis of cell-free DNA (cfDNA) have received much interest due to their promise to identify cancer-causing mutations in the plasma of patients with early stage disease. However, inter- and intra-tumor heterogeneity limit the sensitivity of these methods since recurrent clonal mutations are rare. More recent advances are based on methylation profiling of cfDNA in order to detect and classify reads stemming from a certain tumor type. These approaches are promising but need to be optimized for each tumor type. There is, therefore, a need to provide innovative methods for cancer detection with higher sensitivity due to tumor heterogeneity.

### SUMMARY OF THE INVENTION

Cancer screening methods were discovered by detecting certain pan-cancer methylation signatures of cfDNA. Specifically, the pan-cancer methylation signature is based on loci preferentially methylated in extraembryonic ectoderm that is different from epiblast and that is present across most human cancer types.

Based on these findings, an ultra-sensitive identification of tumor-derived cfDNA was developed that allows non-invasive early diagnosis of human cancer. Computation analysis of methylation haplotypes identified from individual bisulfite-converted reads reduced background signal stemming from normal cell types. The result provides an ability to detect the extraembryonic methylation signature in plasma samples of patients with various stages of cancerous disease. The present invention improves over previous screening methods by providing an ultra-sensitive, non-invasive pan-cancer diagnosis of disease based on plasma cell-free methylation patterns.

In an embodiment, the invention is directed to a method of characterizing a cell-free DNA (cfDNA) sample from a subject, comprising receiving sequencing data comprising reads of methylation sequences for a genomic sequence from the cfDNA sample, wherein the genomic sequence comprises a plurality of CpG Islands (CGI) methylated in the genome of extraembryonic ectoderm (ExE) and not methylated in corresponding epiblast or adult tissue, determining a proportion of haplotypes of the genomic sequence that are fully methylated, and characterizing the cfDNA sample as comprising fully methylated cfCDNA if the proportion of haplotypes is greater than a significance threshold.

In certain embodiments, each haplotype comprises five CGI methylated in the genome of ExE and not methylated in corresponding epiblast or adult tissue. In certain embodiments, the cfDNA sample comprises between 0.01% and 0.1% tumor DNA. In certain embodiments, the sequencing data comprises sequence information for less than 0.3% of the genome of the subject. In certain embodiments, the sequencing data comprises sequence information substantially limited to one or more regions of the subject's genome having a plurality of CGI methylated in the genome of ExE and not methylated in corresponding epiblast or adult tissue. In certain embodiments, the fully methylated haplotypes are compared to one or more pre-established fully methylated haplotype signatures and the cfDNA sample is further characterized as corresponding or not corresponding to the pre-established fully methylated haplotype signature. In certain embodiments, the pre-established fully methylated haplotype signature has been identified via a method comprising random forest, support vector machine, or deep learning analysis. In certain embodiments, the sequencing data comprising reads of methylation sequences for a genomic sequence from the cfDNA sample has been enriched for sequences comprising methylation. In certain embodiments, the enrichment comprises an MBD2 protein-based enrichment method. In certain embodiments, the cfDNA sample was obtained from plasma, urine, stool, menstrual fluid, or lymph fluid. In some embodiments, the method further comprises a step of determining a tissue of origin from the sequencing data.

In an embodiment, the invention is directed to a method for detecting cancer in a subject, comprising receiving sequencing data comprising reads of methylation sequences for a genomic sequence from a cfDNA sample from the subject, wherein the genomic sequence comprises a plurality of CpG Islands (CGI) methylated in the genome of extraembryonic ectoderm (ExE) and not methylated in corresponding epiblast or adult tissue, determining a proportion of haplotypes of the genomic sequence that are fully methylated, and detecting cancer in the subject if the proportion of fully methylated haplotypes is greater than a significance threshold.

In certain embodiments, each haplotype comprises five CGI methylated in the genome of ExE and not methylated in corresponding epiblast or adult tissue. In certain embodiments, the cfDNA sample comprises between 0.01% and 0.1% tumor DNA. In certain embodiments, the sequencing data comprises sequence information for less than 0.3% of the genome of the subject. In certain embodiments, the sequencing data comprises sequence information substantially limited to one or more regions of the subject's genome having a plurality of CGI methylated in the genome of ExE and not methylated in corresponding epiblast or adult tissue. In certain embodiments, the fully methylated haplotypes are compared to one or more pre-established fully methylated haplotype signatures corresponding to one or more tumor types, and the presence or absence of the one or more tumor types are detected in the subject.

In certain embodiments, the one or more tumor types comprise one or more of acute myeloid leukemia, bladder cancer, breast cancer, colon cancer, esophageal cancer, kidney cancer, liver cancer, lung cancer, ovarian cancer, pancreatic cancer, prostate cancer, or stomach cancer. In certain embodiments, the pre-established fully methylated haplotype signatures corresponding to one or more tumor types have been identified via a method comprising random forest, support vector machine, or deep learning analysis. In certain embodiments, the sequencing data comprising reads of methylation sequences for a genomic sequence from the cfDNA sample has been enriched for sequences comprising methylation. In certain embodiments, the enrichment comprises an MBD2 protein-based enrichment method. In certain embodiments, the cfDNA sample was obtained from plasma, urine, stool, menstrual fluid, or lymph fluid. In certain embodiments, the presence of cancer is detected in the sample with 100% sensitivity and 95% specificity. In certain embodiments, the cancer is stage I or stage III. In certain embodiments, the cancer is selected from the group comprising adenocarcinoma, acute myeloid leukemia, bladder cancer, breast cancer, colon cancer, esophageal cancer, kidney cancer, liver cancer, lung cancer, ovarian cancer, pancreatic cancer, prostate cancer, stomach cancer, and uterine cancer. In certain embodiments, the method further comprises a step of treating the subject for cancer when cancer is detected in the subject. In some embodiments, the method further comprises a step of determining a tissue of origin from the sequencing data.

In an embodiment, the invention is directed to a method of detecting eradication of cancer from a subject, comprising receiving sequencing data comprising reads of methylation sequences for a genomic sequence from a cfDNA sample from a subject after a cancer treatment, wherein the genomic sequence comprises a plurality of CGIs methylated in the genome of ExE and not methylated in corresponding epiblast or adult tissue, determining a proportion of haplotypes of the genomic sequence that are fully methylated, and detecting cancer in the subject if the proportion of fully methylated haplotypes is greater than a significance threshold, wherein if cancer is not detected in the subject then the cancer has been eradicated from the subject.

In certain aspects, the genomic sequence comprises a contiguous sequence of about 8 megabases of the human genome comprising a plurality of CGIs methylated in the genome of ExE. In certain embodiments, the genomic sequence comprises a contiguous sequence of about 8 megabases of the human genome comprising a plurality of CGIs methylated in the genome of extraembryonic ectoderm (ExE). In certain embodiments, the genomic sequence comprises 50-75 CGIs methylated in the genome of ExE. In certain embodiments, the genomic sequence comprises a contiguous sequence of about 8 megabases of the human genome comprising a plurality of CGIs methylated in the genome of ExE. In certain embodiments, the genomic sequence comprises 50-75 CGIs methylated in the genome of ExE. In certain aspects, the genomic sequence comprises one or more sequences provided in Table 3.

In an embodiment, the invention is directed to a method of determining a probability distribution of haplotypes comprising receiving sequencing data comprising reads of methylation sequences for a genomic sequence from the cfDNA sample, wherein the genomic sequence comprises a plurality of CpG Islands (CGI) methylated in the genome of extraembryonic ectoderm (ExE) and not methylated in corresponding epiblast or adult tissue, assigning a training or validation set based on the methylated ExE CGI data applying a machine learning method to estimate the probability distribution of all haplotypes across ExE sites, and determining one or more classifications of tumor versus normal samples based on a prediction score obtained from the machine learning method.

In certain embodiments, the machine learning method is random forest. In certain embodiments, the machine learning method is a support vector machine. In certain embodiments, the machine learning method is deep learning. In certain embodiments, the method further comprises the method step of evaluating the performance of the prediction comprising performing an in silico simulation by comparing randomly sampled sequencing reads from epiblast or adult tissue with the ExE reads. In some embodiments, the method further comprises a step of determining a tissue of origin from the sequencing data.

Some aspects of the present disclosure are directed to a method of determining a tissue origin comprising receiving targeted bisulfite sequencing data comprising reads of methylation sequences for a genomic sequence from a cfDNA sample, wherein the genomic sequence comprises a plurality of CpG Islands (CGI) methylated in the genome of extraembryonic ectoderm (ExE) and not methylated in corresponding epiblast or adult, and determining a tissue of origin by calculating a relative abundance of haplotypes from the methylated genomic regions by defining a tissue-specific index (TSI) for each haplotype. In some embodiments, the TSI is calculated by the formula:

$$T S I = \frac{{\sum }_{j = 1}^{n} 1 - \frac{{10}^{PKR ( j )}}{{10}^{PKR ( max )}}}{n - 1}$$

wherein n is the number of tissues, PKR (j) is the fraction of a specific haplomer in tissue, and j and PKR max are PKR of the highest methylated tissue. In some embodiments, the sequencing data comprises one or more sequences provided in Table 2.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A shows Mouse E6.5 conceptus that was used to characterize DNA methylation landscapes of embryonic and extraembryonic tissues by comparing Epiblast and ExE (Extraembryonic Ectoderm).

FIG. 1B shows ExE Hyper CGIs that are genetically more conserved. Mean conservation scores (phyloP30-way) were plotted as a function of distance to center of CGIs. Only CGIs that are close to TSS (+/−2000 bp) were included.

FIG. 1C shows mouse ExE hyper CGIs that were lifted over to orthologous CGIs in human.

FIG. 1D shows ExE hyper CGIs that accurately differentiate cancer from normal samples. 13 TCGA cancer types that contain matched normal tissues were used to test performance of ExE hyper CGIs in cancer prediction. Half samples were randomly chosen to be trained by SVM with Gaussian kernel, the resulting model was used to predict the rest half samples either as tumor or normal. The results were presented as a ROC curve and the area under curve (AUC) is shown.

FIG. 1E shows cancer is genetically heterogeneous and epigenetically homogeneous. The results from FIG. 1D are further summarized to show the fraction of samples in each cancer type that was correctly predicted by ExE hyper CGIs. In parallel, the fraction of samples that contain TP53 mutations are also shown.

FIG. 2A shows an illustration of DNA methylation haplotypes. The methylation pattern of CpGs on each sequencing fragment represents a discrete DNA methylation haplotype, which can be classified as unmethylated reads, discordant reads, or fully methylated reads. Proportion of fully methylated reads (PMR) is defined as fraction of fully methylated reads.

FIG. 2B shows that using proportion of fully methylated reads (PMR) significantly reduces background noise in normal cells. Sequencing reads from public WGBS data at OTX2 locus were aggregated to increase coverage for tumor and normal samples, respectively.

FIG. 2C shows an in silico simulation. Sequencing reads from ExE (tumor-like) were spiked into reads from Epiblast (normal-like). The fraction of ExE-derived reads represent 1%, 0.1% or 0.01% in three sets, respectively. In negative controls, all reads were randomly sampled from Epiblast. Prediction results were shown for PMR, MHL and mean methylation-based methods.

FIG. 3A shows a general workflow of targeted bisulfite sequencing used. MBD enrichment is optional but could be used to specifically enrich methylated reads.

FIG. 3B shows evenness of hybrid capture. On-target coverage was normalized by mean coverage in designed regions. This curve describes the fraction of loci that have coverage higher than pre-defined threshold.

FIG. 3C shows efficiency of targeted sequencing. To assess efficiency of targeted sequencing, the same biological sample was profiled by WGBS and targeted BS. Normalized coverages were shown as a function of distance to center of designed CGIs.

FIG. 3D shows enrichment of methylated haplotypes by proteins with methyl-CpG binding domain (MBD). Enrichment efficiency is measured by proportion of methylated reads.

FIG. 4A shows the correlation of normalized counts between two assays, targeted-BS with and without MBD enrichment. Targeted-BS was performed on 4 samples (HuES64, HCT116, normal uterus and uterus cancer) in two conditions, with or without MBD enrichment. Correlation of normalized counts between two assays were assessed for each type of DNA methylation haplotype. All 32 DNA methylation haplotypes were grouped into 6 classes based on length of fully methylated k-mers.

FIG. 4B shows normalized coverage of fully methylated reads that were compared between two assays, targeted BS with and without MBD enrichment, for uterus cancer and uterus normal. Pearson correlation coefficient is also shown in the figure.

FIG. 4C shows normalized coverage of fully methylated reads were compared between two assays, targeted-BS and WGBS, for uterus cancer and normal uterus. Pearson correlation coefficient is also shown in figure.

FIG. 5A shows ultra-sensitive detection of cancer in a dilution sample of HuES64 DNA mixed with HCT116.

FIG. 5B shows ultra-sensitive detection of cancer in a dilution sample of HuES64 DNA mixed with colon cancer DNA spike-in.

FIG. 5C shows ultra-sensitive detection of cancer in a dilution sample of normal uterus DNA mixed with uterus cancer DNA spike-in. Fractions of spike-in in all three experiments include 1%, 0.1% and 0.01%. NMR-based was used to predict the presense of spike-in using increasing numbers of top ranking markers.

FIG. 6 shows ExE hyper CGIs accurately differentiate cancer from normal samples. 13 TCGA cancer types that contain matched normal tissues were used to test performance of ExE hyper CGIs in cancer prediction. The pan-cancer cohort consists of 685 tumor samples and 710 normal samples, which were subdivided into a training and a validation set with equal sample size. Random forest (RF) was implemented using the ‘randomForest’ function of the ‘randomForest’ R package, using default parameter settings. False positive rate and true positive rate were calculated using the ‘roc’ function of the ‘pROC’ R package, based on the ‘out-of-bag’ votes for the training data. RF was able to classify tumor samples with high specificity, and high sensitivity (AUC=0.98).

FIG. 7 shows a comparison of proportion of fully methylated reads (PMR) with three other metrics used in the literature. Five patterns of methylation haplotype combinations (schematic) are used to illustrate the differences between methylation frequency, number of haplotypes, methylation haplotype load (MHL), and PMR.

FIG. 8 shows a schematic illustration of the method for quantification DNA methylation by PMR. Sixteen DNA methylation haplotypes were shown to represent schematic sequencing reads aligned to a locus. For a DNA methylation haplotype, fully methylated k-mers and total number k-mers were counted for a given width of k-mer. PMR is then defined as proportion of fully methylated k-mer across all reads aligned in a locus.

FIG. 9A shows cancer prediction using mean methylation on simulated data. To evaluate the performance of mean methylation in terms of cancer prediction, in silico simulations were performed by randomly sampling sequencing reads from normal-like tissue epiblast as well as tumor-like tissue ExE as a spike-in. The fraction of spike-in ranged from 0.01% to 1%, which matches the fraction of ctDNA in cell-free DNA. ExE was compared to epiblast to identify CGIs that have higher mean methylation in ExE, as indicated in red.

FIG. 9B shows simulated samples that were compared to epiblast using CGIs defined in previous step, the resulting mean methylation difference was represented as a boxplot for each spike-in group.

FIG. 9C shows the number of CGIs with increased or decreased mean methylation that were counted, respectively, and significance p-value that was estimated by one-sided binomial test to predict presence of ExE DNA.

FIG. 10A shows cancer prediction using MHL on simulated data within silico simulations by randomly sampling sequencing reads from normal-like tissue epiblast as well as tumor-like tissue ExE as a spike-in to evaluate the performance of MHL in terms of cancer prediction. The fraction of spike-in ranged from 0.01% to 1%, which matches the fraction of ctDNA in cell-free DNA. ExE was compared to epiblast to identify CGIs that have higher MHL in ExE, as indicated in red.

FIG. 10B shows simulated samples that were compared to epiblast using CGIs defined in previous step, the resulting MHL difference was represented as a boxplot for each spike-in group.

FIG. 10C shows the number of CGIs with increased or decreased MHL that were counted, respectively, and significance p-value that was estimated by one-sided binomial test to predict presence of ExE DNA.

FIG. 11A shows in silico simulations were performed by randomly sampling sequencing reads from normal-like tissue epiblast as well as tumor-like tissue ExE as a spike-in to evaluate the performance of PMR in terms of cancer prediction. The fraction of spike-in ranged from 0.01% to 1%, which matches the fraction of ctDNA in cell-free DNA. ExE was compared to epiblast to identify CGIs that have higher PMR in ExE, as indicated in red.

FIG. 11B shows simulated samples that were compared to epiblast using CGIs defined in previous step, the resulting PMR difference was represented as a boxplot for each spike-in group.

FIG. 11C shows the number of CGIs with increased or decreased PMR that were counted, respectively, and significance p-value was estimated by one-sided binomial tes to predict presence of ExE DNA.

FIG. 12 shows an identification of optimal k-mer length for PMR. PMR is a function of k-mer length. To identify the optimal k-mer for cancer prediction, simulated data with 0.01% ExE spike-in (Methods) using the PMR method were tested. Maximum sensitivity was achieved when k-mer length was set to 5.

FIG. 13 shows that MHL is a biased metric to measure DNA methylation across assays. Targeted-BS was performed on 4 samples (HuES64, HCT116, uterus cancer and uterus normal tissues) in two conditions, with or without MBD enrichment. MHL were compared between two assays, targeted-BS with and without MBD enrichment, for 4 samples respectively.

FIG. 14 shows PMR is a biased metric to measure DNA methylation across assays. Targeted-BS was performed on 4 samples (HuES64, HCT116, uterus cancer and uterus normal tissues) in two conditions, with or without MBD enrichment. PMR were compared between two assays, targeted-BS with and without MBD enrichment, for 4 samples respectively.

FIG. 15 shows NMR as an unbiased metric to measure DNA methylation across assays. Performance targeted-BS was performed on 4 samples (HuES64, HCT116, uterus cancer and uterus normal tissues) in two conditions, with or without MBD enrichment. NMR were compared between two assays, targeted-BS with and without MBD enrichment, for 4 samples respectively. Pearson correlation coefficient of 0.99 is observed for all 4 samples.

FIG. 16A shows detection of cancer in dilution samples using targeted-BS with MBD enrichment. HuES64 DNA was mixed with HCT116 or colon cancer DNA spike-in, and normal uterus DNA was mixed with uterus cancer DNA spike-in. Fractions of spike-in in all three experiments include 1%, 0.1% and 0.01%. Experiment of FIG. 16A was performed in parallel with 1 μg input DNA.

FIG. 16B shows the parallel experiment with 50 ng DNA. NMR-based was used to predict the presence of spike-in using increasing numbers of top-ranking markers.

FIG. 17A shows an example of how the NMR-based cancer prediction pipeline works on HCT116 dilution data. HCT116 was compared to human ES cell (HuES64) to identify CGIs that have higher NMR in HCT116, with a cutoff of 0.1. Then, these CGIs were ranked descendingly based on the difference of NMR between HCT116 and HuES64. The top 200 CGIs were selected as markers. Scatter plots of NMR are shown in which selected markers were highlighted in red. NMR in test sample was compared to that in HuES64.

FIG. 17B shows boxplots of ΔNMR for 1%, 0.1% and 0.1% spike-in.

FIG. 17C shows, to test whether ΔNMR are statistically higher than zero, the number of markers that were counted with increased NMR (ΔNMR>0), decreased NMR (ΔNMR<0). P-values were calculated by one-sided binomial test.

FIG. 18A shows an example to show how the NMR-based cancer prediction pipeline works on colon cancer dilution data. Colon cancer was compared to normal colon to identify CGIs that have higher NMR in colon cancer, with a cutoff of 0.1. Then, these CGIs were ranked descendingly based on the difference of NMR between tumor samples and Hu64ES. The top 200 CGIs were selected as markers. Scatter plots of NMR (Normal) is shown.

FIG. 18B shows scatter plots of NMR(ES).

FIG. 18C shows boxplots of ΔNMR for 1%, 0.1% and 0.1% spike-in.

FIG. 18D shows, to test whether ΔNMR are statistically higher than zero, the number of markers that were counted with increased NMR (ΔNMR>0), decreased NMR (ΔNMR<0). P-values were calculated by one-sided binomial test.

FIG. 19 shows the identification of optimal k-mer length for NMR. NMR is a function of k-mer length. To identify the optimal k-mer for cancer prediction, colon cancer spike-in data was tested with 0.01% colon cancer DNA. Maximum sensitivity was achieved when k-mer length was set to 5.

FIG. 20A shows detection of cancer in dilution samples using mean methylation. HuES64 DNA was mixed with HCT116 or colon cancer DNA spike-in, and uterus normal DNA was mixed with uterus cancer DNA spike-in. Fractions of spike-in in all three experiments include 1%, 0.1% and 0.01%.

FIG. 20B shows MHL-based method to predict the presence of spike-in using increasing numbers of top-ranking markers.

FIG. 21 shows prediction fraction of tumor DNA in colon cancer cohort. The prediction result was shown for each sample as indicated by the vertical dash line.

FIG. 22 shows the prediction fraction of tumor DNA in breast cancer cohort. Prediction result was shown in figure for each sample as indicated by the vertical dash line.

FIG. 23 shows a diagram of different CGI regions that were analyzed for cancer screening methods.

### DETAILED DESCRIPTION OF THE INVENTION

Methods of Characterizing a Cell-Free DNA (cfDNA) Sample

In one aspect, a method disclosed herein is directed to characterizing a cell-free DNA (cfDNA) sample from a subject, comprising receiving sequencing data comprising reads of methylation sequences for a genomic sequence from the cfDNA sample, wherein the genomic sequence comprises a plurality of CpG Islands (CGI) methylated in the genome of extraembryonic ectoderm (ExE) and not methylated in corresponding epiblast or adult tissue, determining a proportion of haplotypes of the genomic sequence that are fully methylated, and characterizing the cfDNA sample as comprising fully methylated cfCDNA if the proportion of haplotypes is greater than a significance threshold.

In certain aspects, the genomic sequence comprises a contiguous sequence of about 8 megabases of the human genome comprising a plurality of CGIs methylated in the genome of ExE. In certain embodiments, the genomic sequence comprises a contiguous sequence of about 8 megabases of the human genome comprising a plurality of CGIs methylated in the genome of ExE and comprising bases 57,258,577-57,282,377 of chr14 (human). In certain embodiments, the genomic sequence comprises a contiguous sequence of up to 8 megabases of the human genome comprising a plurality of CGIs methylated in the genome of extraembryonic ectoderm (ExE). In certain embodiments, the genomic sequence comprises a contiguous sequence of 6.1 megabases of the human genome comprising a plurality of CGIs methylated in the genome of extraembryonic ectoderm (ExE). In certain aspects, the genomic sequence comprises one or more sequences provided in Table 3.

In certain embodiments, the genomic sequence comprises 50-75 CGIs methylated in the genome of ExE. In certain embodiments, the genomic sequence comprises a contiguous sequence of about 8 megabases of the human genome comprising a plurality of CGIs methylated in the genome of ExE. In certain embodiments, the genomic sequence comprises 50-75 CGIs methylated in the genome of ExE. In certain embodiments, the genomic sequence comprises up to 100 CGIs methylated in the genome of ExE. In certain embodiments, the genomic sequence comprises up to 500 CGIs methylated in the genome of ExE. In certain embodiments, the genomic sequence comprises up to 1000 CGIs methylated in the genome of ExE. In certain embodiments, the genomic sequence comprises up to 1500 CGIs methylated in the genome of ExE. In a more particular embodiment, the genomic sequence comprises about 1,265 CGIs hypermethylated in ExE tissues. In a more particular embodiment, the genomic sequence comprises about 473 CGIs hypermethylated in ExE tissues.

As used herein, the significance threshold refers to an observed significance value known as a significance prediction value (p-value) estimated by a one-sided binomial test to predict presence of ExE DNA. In certain embodiments, for a 5% fraction of ctDNA in cell-free DNA the P-value (i.e., the minimum p-value signifying significance) is 5.3×10⁻¹⁴⁵. In certain embodiments, for a 1% fraction of ctDNA in cell-free DNA the P-value is 3.9×10⁻⁷⁸. In certain embodiments, for a 0.1% fraction of ctDNA in cell-free DNA the P-value is 6.5×10⁻¹⁹. In certain embodiments, for a 0.01% fraction of ctDNA in cell-free DNA the P-value is 6.3×10⁻⁴. In certain embodiments, for a 5% fraction of ctDNA in cell-free DNA the P-value is 1.9×10⁻⁷⁸. In certain embodiments, for a 1% fraction of ctDNA in cell-free DNA the P-value is 7.4×10⁻³⁴. In certain embodiments, for a 0.1% fraction of ctDNA in cell-free DNA the P-value is 4.2×10⁻¹⁰. In certain embodiments, for a 0.01% fraction of ctDNA in cell-free DNA the P-value is 3.1×10⁻². In certain embodiments, for a 5% fraction of ctDNA in cell-free DNA the P-value is 4.5×10⁻²⁶. In certain embodiments, for a 1% fraction of ctDNA in cell-free DNA the P-value is 3.4×10⁻¹⁵. In certain embodiments, for a 0.1% fraction of ctDNA in cell-free DNA the P-value is 1.1×10⁻⁸. In certain embodiments, for a 0.01% fraction of ctDNA in cell-free DNA the P-value is 4.5×10⁻⁶. In certain embodiments, at a 1% fraction, the P-value is 1.3×10⁻⁵⁸. In certain embodiments, at a 0.1% fraction, the P-value is 2.0×10⁻³⁷. In certain embodiments, at a 0.01% fraction, the P-value is 3.9×10⁻⁹. In certain embodiments, at a 1% fraction, the P-value is 1.6×10⁻⁵⁴. In certain embodiments, at a 0.1% fraction, the P-value is 3.3×10⁻²⁶. In certain embodiments, at a 0.01% fraction, the P-value is 1.1×10⁻⁵.

In certain aspects, the cfDNA sample comprises between 0.01% and 0.1% of tumor DNA. In certain aspects, the cfDNA sample comprises 0.01% of tumor DNA. In certain aspects, the cfDNA sample comprises 0.02% of tumor DNA. In certain aspects, the cfDNA sample comprises 0.03% of tumor DNA. In certain aspects, the cfDNA sample comprises 0.04% of tumor DNA. In certain aspects, the cfDNA sample comprises 0.05% of tumor DNA. In certain aspects, the cfDNA sample comprises 0.06% of tumor DNA. In certain aspects, the cfDNA sample comprises 0.07% of tumor DNA. In certain aspects, the cfDNA sample comprises 0.08% of tumor DNA. In certain aspects, the cfDNA sample comprises 0.09% of tumor DNA. In certain aspects, the cfDNA sample comprises 0.1% of tumor DNA. In certain aspects, the cfDNA sample comprises 0.15% of tumor DNA. In certain aspects, the cfDNA sample comprises 0.2% of tumor DNA. In certain aspects, the cfDNA sample comprises 0.25% of tumor DNA. In certain aspects, the cfDNA sample comprises 0.3% of tumor DNA. In certain aspects, the cfDNA sample comprises 0.35% of tumor DNA. In certain aspects, the cfDNA sample comprises 0.25% of tumor DNA. In certain aspects, the cfDNA sample comprises 0.3% of tumor DNA. In certain aspects, the cfDNA comprises 0.4% of tumor DNA. In certain aspects, the cfDNA comprises 0.5% or more of tumor DNA. In certain aspects, the cfDNA comprises 1% or more of tumor DNA. In certain aspects, the cfDNA comprises 1.5% or more of tumor DNA. In certain aspects, the cfDNA comprises 2% or more of tumor DNA. In certain aspects, the cfDNA comprises 3% or more of tumor DNA. In certain aspects, the cfDNA comprises 4% or more of tumor DNA. In certain aspects, the cfDNA comprises 5% or more of tumor DNA.

In certain aspects, the sequencing data comprises sequence information for less than 0.01% of the genome of the subject. In certain aspects, the sequencing data comprises sequence information for less than 0.05% of the genome of the subject. In certain aspects, the sequencing data comprises sequence information for less than 0.1% of the genome of the subject. In certain aspects, the sequencing data comprises sequence information for less than 0.2% of the genome of the subject. In certain aspects, the sequencing data comprises sequence information for less than 0.3% of the genome of the subject. In certain aspects, the sequencing data comprises sequence information for less than 0.4% of the genome of the subject. In certain aspects, the sequencing data comprises sequence information for less than 0.5% of the genome of the subject. In certain aspects, the sequencing data comprises sequence information for less than 0.6% of the genome of the subject. In certain aspects, the sequencing data comprises sequence information for less than 0.7% of the genome of the subject. In certain aspects, the sequencing data comprises sequence information for less than 0.8% of the genome of the subject. In certain aspects, the sequencing data comprises sequence information for less than 0.9% of the genome of the subject. In certain aspects, the sequencing data comprises sequence information for less than 1% of the genome of the subject. In certain aspects, the sequencing data comprises sequence information for less than 1.1% of the genome of the subject. In certain aspects, the sequencing data comprises sequence information for less than 1.2% of the genome of the subject. In certain aspects, the sequencing data comprises sequence information for less than 1.3% of the genome of the subject. In certain aspects, the sequencing data comprises sequence information for less than 1.4% of the genome of the subject. In certain aspects, the sequencing data comprises sequence information for less than 1.5% of the genome of the subject. In certain aspects, the sequencing data comprises sequence information for less than 1.6% of the genome of the subject. In certain aspects, the sequencing data comprises sequence information for less than 1.7% of the genome of the subject. In certain aspects, the sequencing data comprises sequence information for less than 1.8% of the genome of the subject. In certain aspects, the sequencing data comprises sequence information for less than 1.9% of the genome of the subject. In certain aspects, the sequencing data comprises sequence information for less than 2% of the genome of the subject. In certain aspects, the sequencing data comprises sequence information for less than 5% of the genome of the subject. In certain aspects, the sequencing data comprises sequence information for less than 10% of the genome of the subject.

In certain aspects, each haplotype comprises five CGI methylated in the genome of ExE not methylated in corresponding epiblast or adult tissue. In certain aspects, each haplotype comprises four CGI methylated in the genome of ExE not methylated in corresponding epiblast or adult tissue. In certain aspects, each haplotype comprises three CGI methylated in the genome of ExE not methylated in corresponding epiblast or adult tissue. In certain aspects, each haplotype comprises two CGI methylated in the genome of ExE not methylated in corresponding epiblast or adult tissue. In certain aspects, each haplotype comprises one CGI methylated in the genome of ExE not methylated in corresponding epiblast or adult tissue. In certain aspects, each haplotype comprises six CGI methylated in the genome of ExE not methylated in corresponding epiblast or adult tissue. In certain aspects, each haplotype comprises seven CGI methylated in the genome of ExE not methylated in corresponding epiblast or adult tissue. In certain aspects, each haplotype comprises eight CGI methylated in the genome of ExE not methylated in corresponding epiblast or adult tissue. In certain aspects, each haplotype comprises nine CGI methylated in the genome of ExE not methylated in corresponding epiblast or adult tissue. In certain aspects, each haplotype comprises ten CGI methylated in the genome of ExE not methylated in corresponding epiblast or adult tissue.

In certain aspects, the sequencing data comprises sequence information substantially limited to one or more regions of the subject's genome having a plurality of CGI methylated in the genome of ExE and is not methylated in corresponding epiblast or adult tissue. In certain aspects, the one or more regions of the subject genome are about 1200 CGIs as a pan-cancer methylation signature (e.g., as shown in Table 3). In certain aspects, the one or more regions are one to five CGI patterns representing a discrete DNA methylation haplotype. In certain aspects, the region is an 8 megabase region. In certain aspects, the 8 megabase region comprises CHR14: 57,258,577-57,282,337. In certain aspects, the genomic regions comprise one or more sequences provided in Table 3.

In certain aspects, fully methylated haplotypes are compared to one or more pre-established fully methylated haplotype signatures. The cfDNA sample is further characterized as corresponding or not corresponding to the pre-established fully methylated haplotype signature. In some embodiments, the fully methylated haplotypes are globally normalized for the number of haplotypes in a region by total number of haplotypes across all regions (i.e., to obtain an NMR).

In certain aspects, the pre-established fully methylated haplotype signature has been identified via a method comprising random forest, support vector machine, or deep learning analysis. As used herein, random forest algorithm operates by constructing a multitude of decision trees at training time and outputting the classification or mean/average prediction/regression of the individual trees.

As used herein, support vector machine is a machine learning method that constructs a set of hyperplanes that can be used for classification, regression, or detection of multidimensional data. As used herein, deep learning analysis refers to a class of machine learning algorithms that use multiple layers to progressively extract higher-level features from the raw input.

In certain aspects, the sequencing data includes reads of methylation sequences for a genomic sequence from the cfDNA sample that has been enriched for methylation sequences. In certain aspects, the enrichment includes a methyl-DNA binding protein-based enrichment method. In certain aspects, the methyl-DNA binding protein of the enrichment method is a methyl-binding domain (MBD) selected from MBD1, MBD2, MBD3, and MBD4

As used herein, “sample” is not limited and may be any suitable fluid disclosed herein. In some embodiments, the sample is blood, serum, plasma, urine, stool, menstrual fluid, lymph fluid, and other bodily fluids.

As used herein, “CpG” and “CpG dinucleotide” are used interchangeably and refer to a dinucleotide sequence containing an adjacent guanine and cytosine where the cytosine is located 5′ of guanine.

As used herein, “CpG island” or “CGI” refers to a region with a high frequency of CpG sites. The region is at least 200 bp, with a GC percentage greater than 50%, and an observed-to-expected CpG ratio greater than 60%.

As used herein, a “haplotype” refers to a combination of CpG sites found on the same chromosome. Similarly, a “DNA methylation haplotype” represents the DNA methylation status of CpG sites on the same chromosome.

In certain embodiments, a sample (e.g., a fluid sample) is screened using whole-genome bisulfite sequencing (WGBS), TCGA Illumina Infinium HumanMethylation450K BeadChip sequencing (TCGA), and/or reduced representation bisulfite sequencing (RRBS), or by other suitable methylation detection assays known in the art.

In certain embodiments, the inventions disclosed herein relate to methods of using proportion of concordantly methylated reads (PMR) (i.e., fully methylated haplotypes) to detect circulating tumor DNA (ctDNA) in a sample. In certain aspects, a methylation sequence for a sample is obtained and at least one CpG Island (CGI) is identified on the methylation sequence. PMR for the identified CpG Island is calculated and then compared to a control background of a normal tissue or epiblast. The presence of ctDNA is detected in the sample when the PMR of the sample is larger than the control background (e.g., signal is higher by bank sum test).

The presence of ctDNA may be detected in the cfDNA with a greater sensitivity and specificity than methods previously known by those of skill in the art. For example, ctDNA may be detected in the sample using PMR with a sensitivity of greater than 75%, 80%, 85%, 90%, 95%, or 99%. In certain aspects, ctDNA is detected in the sample using PMR with 100% sensitivity. ctDNA may be detected in the sample using PMR with a specificity of greater than 50%, 55%, 60%, 65%, 70%, 75%, 80%, 85%, 90%, or 95%. In certain aspects, ctDNA is detected in the sample using PMR with 95% specificity. In some aspects, ctDNA is detected in the sample using PMR with at least 90% sensitivity and at least 90% specificity. In some aspects, ctDNA is detected in the sample using PMR with at least 100% sensitivity and at least 95% specificity.

As used herein, “sensitivity” measures the proportion of positives (i.e., the presence of ctDNA) that are correctly identified in the cfDNA.

As used herein, “specificity” measures the proportion of negatives (i.e., non-ctDNA) that are correctly identified in the cfDNA.

The amount of ctDNA detected in the sample may be measured and quantified. In some aspects, the sample comprises 0.005% to 1.5% ctDNA, 0.01% to 1% ctDNA, 0.05% to 0.5% ctDNA, 0.1% to 0.3% ctDNA. In some embodiments, the sample comprises 0.01% ctDNA. In certain aspects, the presence of 0.01% ctDNA is detected in cfDNA using PMR with about 100% sensitivity and about 95% specificity, with a p-value cutoff of 10⁻⁴.

In some embodiments, the inventions disclosed herein relate to methods of screening for cancer by using PMR to detect ctDNA in a sample as described herein, wherein the presence of ctDNA in the sample is indicative of the subject having cancer.

The methods described herein may be applied to a subject who is at risk of cancer or at risk of cancer recurrence. The subject is not limited and may be any suitable subject. In some embodiments, the subject is an individual diagnosed with, suffering from, at risk of developing, or suspected of having cancer. In some embodiments, the subject is a human. In some embodiments, the subject is a non-human mammal. In some embodiments, the subject is a non-mammal vertebrate animal. In some embodiments, the subject is a common lab animal. A subject at risk of cancer may be, e.g., a subject who has not been diagnosed with cancer but has an increased risk of developing cancer. Determining whether a subject is considered “at increased risk” of cancer is within the skill of the ordinarily skilled medical practitioner. Any suitable test(s) and/or criteria can be used. For example, a subject may be considered “at increased risk” of developing cancer if any one or more of the following apply: (i) the subject has an inherited mutation or genetic polymorphism that is associated with increased risk of developing or having cancer relative to other members of the general population not having such mutation or genetic polymorphism (e.g., inherited mutations in certain TSGs are known to be associated with increased risk of cancer); (ii) the subject has a gene or protein expression profile, and/or presence of particular substance(s) in a sample obtained from the subject (e.g., blood), that is/are associated with increased risk of developing or having cancer relative to the general population; (iii) the subject has one or more risk factors such as a family history of cancer, exposure to a tumor-promoting agent or carcinogen (e.g., a physical carcinogen, such as ultraviolet or ionizing radiation; a chemical carcinogen such as asbestos, tobacco or smoke components, aflatoxin, arsenic; a biological carcinogen such as certain viruses or parasites); (iv) the subject is over a specified age, e.g., over 60 years of age. A subject suspected of having cancer may be a subject who has one or more symptoms of cancer or who has had a diagnostic procedure performed that suggested or was consistent with the possible existence of cancer. A subject at risk of cancer recurrence may be a subject who has been treated for cancer and appears to be free of cancer, e.g., as assessed by an appropriate method.

As used herein, the phrase “cancer” is intended to broadly apply to any cancerous condition.

In certain aspects, the cancer is stage I, stage II, stage III, or stage IV. In certain aspects, the cancerous cells are present but have not spread to nearby tissue.

Illustrative examples of cancers include, but are not limited to, adrenal cancer, adrenocortical carcinoma, anal cancer, appendix cancer, astrocytoma, atypical teratoid/rhabdoid tumor, basal cell carcinoma, bile duct cancer, bladder cancer, bone cancer, brain/CNS cancer, breast cancer, bronchial tumors, cardiac tumors, cervical cancer, cholangiocarcinoma, chondrosarcoma, chordoma, colon cancer, colorectal cancer, craniopharyngioma, ductal carcinoma in situ (DCIS) endometrial cancer, ependymoma, esophageal cancer, esthesioneuroblastoma, Ewing's sarcoma, extracranial germ cell tumor, extragonadal germ cell tumor, eye cancer, fallopian tube cancer, fibrous histiosarcoma, fibrosarcoma, gallbladder cancer, gastric cancer, gastrointestinal carcinoid tumors, gastrointestinal stromal tumor (GIST), germ cell tumors, glioma, glioblastoma, head and neck cancer, hemangioblastoma, hepatocellular cancer, hypopharyngeal cancer, intraocular melanoma, kaposi sarcoma, kidney cancer, laryngeal cancer, leiomyosarcoma, lip cancer, liposarcoma, liver cancer, lung cancer, non-small cell lung cancer, lung carcinoid tumor, malignant mesothelioma, medullary carcinoma, medulloblastoma, menangioma, melanoma, Merkel cell carcinoma, midline tract carcinoma, mouth cancer, myxosarcoma, myelodysplastic syndrome, myeloproliferative neoplasms, nasal cavity and paranasal sinus cancer, nasopharyngeal cancer, neuroblastoma, oligodendroglioma, oral cancer, oral cavity cancer, oropharyngeal cancer, osteosarcoma, ovarian cancer, pancreatic cancer, pancreatic islet cell tumors, papillary carcinoma, paraganglioma, parathyroid cancer, penile cancer, pharyngeal cancer, pheochromocytoma, pinealoma, pituitary tumor, pleuropulmonary blastoma, primary peritoneal cancer, prostate cancer, rectal cancer, retinoblastoma, renal cell carcinoma, renal pelvis and ureter cancer, rhabdomyosarcoma, salivary gland cancer, sebaceous gland carcinoma, skin cancer, soft tissue sarcoma, squamous cell carcinoma, small cell lung cancer, small intestine cancer, stomach cancer, sweat gland carcinoma, synovioma, testicular cancer, throat cancer, thymus cancer, thyroid cancer, urethral cancer, uterine cancer, uterine sarcoma, vaginal cancer, vascular cancer, vulvar cancer, and Wilms Tumor. In some embodiments of the methods described herein, the cancer is adrenocortical carcinoma, bladder urothelial carcinoma, breast invasive carcinoma, cervical and endocervical cancers, cholangiocarcinoma, colon adenocarcinoma, colorectal adenocarcinoma, lymphoid neoplasm diffuse large B-cell lymphoma, esophageal carcinoma, FFPE Pilot Phase II, glioblastoma multiforme, glioma, head and neck squamous cell carcinoma, kidney chromophobe, pan-kidney cohort (KICH+KIRC+KIRP), kidney renal clear cell carcinoma, kidney renal papillary cell carcinoma, acute myeloid leukemia, brain lower grade glioma, liver hepatocellular carcinoma, lung adenocarcinoma, lung squamous cell carcinoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, stomach and esophageal carcinoma, testicular germ cell tumors, thyroid carcinoma, thymoma, uterine corpus endometrial carcinoma, uterine carcinosarcoma, and uveal melanoma. In other embodiments, the invention provides methods of treating a subject in need of treatment for cancer.

In some embodiments, PMR is used to detect ctDNA in a sample as described herein, where the presence of the ctDNA is indicative of the subject having cancer. The individual is then treated for cancer using any methods of treatment generally known to those of skill in the art (e.g., therapeutics or procedures).

For example, therapies or anticancer agents that may be used for treating the subject include anti-cancer agents, chemotherapeutic drugs, surgery, radiotherapy (e.g., γ-radiation, neutron beam radiotherapy, electron beam radiotherapy, proton therapy, brachytherapy, and systemic radioactive isotopes), endocrine therapy, biologic response modifiers (e.g., interferons, interleukins), hyperthermia, cryotherapy, agents to attenuate any adverse effects, or combinations thereof, useful for treating a subject in need of treatment for a cancer. Non-limiting examples of cancer chemotherapeutic agents that may be used include, e.g., alkylating and alkylating-like agents such as nitrogen mustards (e.g., chlorambucil, chlormethine, cyclophosphamide, ifosfamide, and melphalan), nitrosoureas (e.g., carmustine, fotemustine, lomustine, streptozocin); platinum agents (e.g., alkylating-like agents such as carboplatin, cisplatin, oxaliplatin, BBR3464, satraplatin), busulfan, dacarbazine, procarbazine, temozolomide, thioTEPA, treosulfan, and uramustine; antimetabolites such as folic acids (e.g., aminopterin, methotrexate, pemetrexed, raltitrexed); purines such as cladribine, clofarabine, fludarabine, mercaptopurine, pentostatin, thioguanine; pyrimidines such as capecitabine, cytarabine, fluorouracil, floxuridine, gemcitabine; spindle poisons/mitotic inhibitors such as taxanes (e.g., docetaxel, paclitaxel), vincas (e.g., vinblastine, vincristine, vindesine, and vinorelbine), epothilones; cytotoxic/anti-tumor antibiotics such anthracyclines (e.g., daunorubicin, doxorubicin, epirubicin, idarubicin, mitoxantrone, pixantrone, and valrubicin), compounds naturally produced by various species of Streptomyces (e.g., actinomycin, bleomycin, mitomycin, plicamycin) and hydroxyurea; topoisomerase inhibitors such as camptotheca (e.g., camptothecin, topotecan, irinotecan) and podophyllums (e.g., etoposide, teniposide); monoclonal antibodies for cancer therapy such as anti-receptor tyrosine kinases (e.g., cetuximab, panitumumab, trastuzumab), anti-CD20 (e.g., rituximab and tositumomab), and others for example alemtuzumab, aevacizumab, gemtuzumab; photosensitizers such as aminolevulinic acid, methyl aminolevulinate, porfimer sodium, and verteporfin; tyrosine and/or serine/threonine kinase inhibitors, e.g., inhibitors of Abl, Kit, insulin receptor family member(s), VEGF receptor family member(s), EGF receptor family member(s), PDGF receptor family member(s), FGF receptor family member(s), mTOR, Raf kinase family, phosphatidyl inositol (PI) kinases such as PI3 kinase, PI kinase-like kinase family members, cyclin dependent kinase (CDK) family members, Aurora kinase family members (e.g., kinase inhibitors that are on the market or have shown efficacy in at least one phase III trial in tumors, such as cediranib, crizotinib, dasatinib, erlotinib, gefitinib, imatinib, lapatinib, nilotinib, sorafenib, sunitinib, vandetanib), growth factor receptor antagonists, and others such as retinoids (e.g., alitretinoin and tretinoin), altretamine, amsacrine, anagrelide, arsenic trioxide, asparaginase (e.g., pegasparagase), bexarotene, bortezomib, denileukin diftitox, estramustine, ixabepilone, masoprocol, mitotane, and testolactone, Hsp90 inhibitors, proteasome inhibitors (e.g., bortezomib), angiogenesis inhibitors, e.g., anti-vascular endothelial growth factor agents such as bevacizumab (Avastin) or VEGF receptor antagonists, matrix metalloproteinase inhibitors, various pro-apoptotic agents (e.g., apoptosis inducers), Ras inhibitors, anti-inflammatory agents, cancer vaccines, or other immunomodulating therapies, etc. It will be understood that the preceding classification is non-limiting.

In some embodiments, the method further comprises a step of determining a tissue of origin from the sequencing data.

### Methods for Detecting Cancer

In another aspect, a method as described herein is directed to a method for detecting cancer in a subject comprising receiving sequencing data comprising reads of methylation sequences for a genomic sequence from a cfDNA sample from the subject wherein the genomic sequence comprises a plurality of CpG Islands (CGI) methylated in the genome of extraembryonic ectoderm (ExE) and that are not methylated in corresponding epiblast or adult tissue, determining a proportion of haplotypes of the genomic sequence that are fully methylated, and detecting cancer in the subject if the proportion of fully methylated haplotypes is greater than a significance threshold.

The cancer is not limited and may be any cancer described herein. In certain aspects, the cancer is selected from acute myeloid leukemia, bladder cancer, breast cancer, colon cancer, esophageal cancer, kidney cancer, liver cancer, lung cancer, ovarian cancer, pancreatic cancer, prostate cancer, and stomach cancer.

In certain aspects, each haplotype comprises five CGI methylated in the genome of ExE not methylated in corresponding epiblast or adult tissue. In certain aspects, each haplotype comprises four CGI methylated in the genome of ExE not methylated in corresponding epiblast or adult tissue. In certain aspects, each haplotype comprises three CGI methylated in the genome of ExE not methylated in corresponding epiblast or adult tissue. In certain aspects, each haplotype comprises two CGI methylated in the genome of ExE not methylated in corresponding epiblast or adult tissue. In certain aspects, each haplotype comprises one CGI methylated in the genome of ExE not methylated in corresponding epiblast or adult tissue. In certain aspects, each haplotype comprises six CGI methylated in the genome of ExE not methylated in corresponding epiblast or adult tissue. In certain aspects, each haplotype comprises seven CGI methylated in the genome of ExE not methylated in corresponding epiblast or adult tissue. In certain aspects, each haplotype comprises eight CGI methylated in the genome of ExE not methylated in corresponding epiblast or adult tissue. In certain aspects, each haplotype comprises nine CGI methylated in the genome of ExE not methylated in corresponding epiblast or adult tissue. In certain aspects, each haplotype comprises ten CGI methylated in the genome of ExE not methylated in corresponding epiblast or adult tissue.

In certain aspects, the cfDNA sample comprises between 0.01% and 0.1% of tumor DNA. In certain aspects, the cfDNA sample comprises 0.01% of tumor DNA. In certain aspects, the cfDNA sample comprises 0.02% of tumor DNA. In certain aspects, the cfDNA sample comprises 0.03% of tumor DNA. In certain aspects, the cfDNA sample comprises 0.04% of tumor DNA. In certain aspects, the cfDNA sample comprises 0.05% of tumor DNA. In certain aspects, the cfDNA sample comprises 0.06% of tumor DNA. In certain aspects, the cfDNA sample comprises 0.07% of tumor DNA. In certain aspects, the cfDNA sample comprises 0.08% of tumor DNA. In certain aspects, the cfDNA sample comprises 0.09% of tumor DNA. In certain aspects, the cfDNA sample comprises 0.1% of tumor DNA.

In certain aspects, the sequencing data comprises sequence information for less than 0.1% of the genome of the subject. In certain aspects, the sequencing data comprises sequence information for less than 0.2% of the genome of the subject. In certain aspects, the sequencing data comprises sequence information for less than 0.3% of the genome of the subject. In certain aspects, the sequencing data comprises sequence information for less than 0.4% of the genome of the subject. In certain aspects, the sequencing data comprises sequence information for less than 0.5% of the genome of the subject. In certain aspects, the sequencing data comprises sequence information for less than 0.6% of the genome of the subject. In certain aspects, the sequencing data comprises sequence information for less than 0.7% of the genome of the subject. In certain aspects, the sequencing data comprises sequence information for less than 0.8% of the genome of the subject. In certain aspects, the sequencing data comprises sequence information for less than 0.9% of the genome of the subject. In certain aspects, the sequencing data comprises sequence information for less than 1% of the genome of the subject. In certain aspects, the sequencing data comprises sequence information for less than 1.1% of the genome of the subject. In certain aspects, the sequencing data comprises sequence information for less than 1.2% of the genome of the subject. In certain aspects, the sequencing data comprises sequence information for less than 1.3% of the genome of the subject. In certain aspects, the sequencing data comprises sequence information for less than 1.4% of the genome of the subject. In certain aspects, the sequencing data comprises sequence information for less than 1.5% of the genome of the subject. In certain aspects, the sequencing data comprises sequence information for less than 1.6% of the genome of the subject. In certain aspects, the sequencing data comprises sequence information for less than 1.7% of the genome of the subject. In certain aspects, the sequencing data comprises sequence information for less than 1.8% of the genome of the subject. In certain aspects, the sequencing data comprises sequence information for less than 1.9% of the genome of the subject. In certain aspects, the sequencing data comprises sequence information for less than 2% of the genome of the subject.

In certain aspects, the sequencing data comprises sequence information substantially limited to one or more regions of the subject's genome having a plurality of CGI methylated in the genome of ExE and not methylated in corresponding epiblast or adult tissue.

In certain aspects, fully methylated haplotypes are compared to one or more pre-established fully methylated haplotype signatures corresponding to one or more tumor types. The method includes determining the presence or absence of the one or more tumor types that are detected in the subject.

In certain aspects, the pre-established fully methylated haplotype signatures corresponding to one or more tumor types have been identified via a method comprising random forest, support vector machine, or deep learning analysis.

In certain aspects, the sequencing data comprising reads of methylation sequences for a genomic sequence from the cfDNA sample has been enriched for sequences comprising methylation. In certain aspects, the enrichment includes a methyl-DNA binding protein-based enrichment method. In certain aspects, the methyl-DNA binding protein of the enrichment method is a methyl-binding domain (MBD) selected from MBD1, MBD2, MBD3, and MBD4. In certain aspects, the enrichment method further comprises targeted bisulfite sequencing (targeted-BS). In certain aspects, up to 6.2 Mb of ExE hyper CGIs are enriched. In certain aspects, the enrichment method achieved greater than 50-fold enrichment compared to whole-genome bisulfite sequencing (WGBS). In certain aspects, the enrichment method achieved greater than 100-fold enrichment compared to WGBS. In certain aspects, the enrichment method achieved greater than 400-fold enrichment compared to WGBS.

In certain aspects, the cfDNA sample was obtained from plasma, urine, stool, menstrual fluid, or lymph fluid.

In certain aspects, the presence of cancer is detected in the sample with 100% sensitivity and 95% specificity. The presence of ctDNA may be detected in the cfDNA with a greater sensitivity and specificity than methods previously known by those of skill in the art. For example, ctDNA may be detected in the sample using PMR with a sensitivity of greater than 75%, 80%, 85%, 90%, 95%, or 99%. In certain aspects, ctDNA is detected in the sample using PMR with 100% sensitivity. ctDNA may be detected in the sample using PMR with a specificity of greater than 50%, 55%, 60%, 65%, 70%, 75%, 80%, 85%, 90%, or 95%. In certain aspects, ctDNA is detected in the sample using PMR with 95% specificity. In some aspects, ctDNA is detected in the sample using PMR with at least 90% sensitivity and at least 90% specificity. In some aspects, ctDNA is detected in the sample using PMR with at least 100% sensitivity and at least 95% specificity.

In certain aspects, the method further includes the step of treating the subject for cancer when cancer is detected in the subject. The method of treating is not limited and may be any method described herein. In some embodiments, the method of treating is with a chemotherapeutic agent. In some embodiments, the method further comprises a step of determining a tissue of origin from the sequencing data.

### Methods of Detecting Eradication of Cancer

In another aspect, a method disclosed herein is directed to detecting eradication of a cancer from a subject, comprising receiving sequencing data comprising reads of methylation sequences for a genomic sequence from a cfDNA sample from a subject after a cancer treatment, wherein the genomic sequence comprises a plurality of CpG Islands (CGI) methylated in the genome of extraembryonic ectoderm (ExE) and not methylated in corresponding epiblast or adult tissue, determining a proportion of haplotypes of the genomic sequence that are fully methylated, and detecting cancer in the subject if the proportion of fully methylated haplotypes is greater than a significance threshold, wherein if cancer is not detected in the subject then the cancer has been eradicated from the subject. The cancer is not limited and may be any suitable cancer described herein. The subject is not limited and also may be any subject described herein. In some aspects, the subject is human.

In certain aspects, the genomic sequence comprises 1-1300 CGIs methylated in the genome of ExE. In certain aspects, the genomic sequence comprises 1-25 CGIs methylated in the genome of ExE. In certain aspects, the genomic sequence comprises 25-50 CGIs methylated in the genome of ExE. In certain aspects, the genomic sequence comprises 50-75 CGIs methylated in the genome of ExE. In certain aspects, the genomic sequence comprises 50-75 CGIs methylated in the genome of ExE. In certain aspects, the genomic sequence comprises 75-100 CGIs methylated in the genome of ExE. In certain aspects, the genomic sequence comprises 100-200 CGIs methylated in the genome of ExE. In certain aspects, the genomic sequence comprises 200-300 CGIs methylated in the genome of ExE. In certain aspects, the genomic sequence comprises 300-400 CGIs methylated in the genome of ExE. In certain aspects, the genomic sequence comprises 400-500 CGIs methylated in the genome of ExE. In certain aspects, the genomic sequence comprises 500-600 CGIs methylated in the genome of ExE. In certain aspects, the genomic sequence comprises 600-700 CGIs methylated in the genome of ExE. In certain aspects, the genomic sequence comprises 700-800 CGIs methylated in the genome of ExE. In certain aspects, the genomic sequence comprises 800-900 CGIs methylated in the genome of ExE. In certain aspects, the genomic sequence comprises 900-1000 CGIs methylated in the genome of ExE. In certain aspects, the genomic sequence comprises 1000-1100 CGIs methylated in the genome of ExE. In certain aspects, the genomic sequence comprises 1100-1200 CGIs methylated in the genome of ExE. In certain aspects, the genomic sequence comprises 1200-1300 CGIs methylated in the genome of ExE.

As used herein, eradication of the cancer refers to a substantial reduction in cancerous cells as compared to an original sample. In certain embodiments, the substantial reduction means a reduction of 90% or more of cancerous cells. In certain embodiments, the substantial reduction means a reduction of 95% or more of cancerous cells. In certain embodiments, the substantial reduction means a reduction of 98% or more of cancerous cells. In certain embodiments, the substantial reduction means a reduction of 99% or more of cancerous cells. In certain embodiments, the substantial reduction means a reduction of 99.5% or more of cancerous cells. In certain embodiments, the substantial reduction means a reduction of 99.9% or more of cancerous cells. In certain embodiments, the substantial reduction means a reduction of 99.99% or more of cancerous cells. In certain embodiments, the substantial reduction means a reduction of 99.999% or more of cancerous cells. In certain embodiments, the substantial reduction means a reduction of 100% of cancerous cells. In certain embodiments, the substantial reduction means only a trace amount cancerous cells exist.

### Methods for Determining Probability Distribution

In another aspect, the invention is directed to a method of determining a probability distribution of haplotypes comprising the steps of receiving sequencing data comprising reads of methylation sequences for a genomic sequence from the cfDNA sample, wherein the genomic sequence comprises a plurality of CpG Islands (CGI) methylated in the genome of extraembryonic ectoderm (ExE) and not methylated in corresponding epiblast or adult tissue, assigning a training or validation set based on the methylated ExE CGI data applying a machine learning method to estimate the probability distribution of all haplotypes across ExE sites, and determining one or more classifications of tumor versus normal samples based on a prediction score (P-score) as used herein is obtained from the machine learning method.

In certain aspects, the machine learning method is random forest. In certain aspects, the machine learning method is a support vector machine. In certain aspects, the machine learning method is deep learning.

In certain aspects, the above methods further include a method of evaluating the performance of the prediction comprising performing an in silico simulation by comparing randomly sampled sequencing reads from epiblast or adult tissue with the ExE reads. In some embodiments, the method further comprises a step of determining a tissue of origin from the sequencing data.

### Determining Tissue of Origin

Some aspects of the present disclosure are directed to a method of determining a tissue origin comprising receiving targeted bisulfite sequencing data comprising reads of methylation sequences for a genomic sequence from a cfDNA sample, wherein the genomic sequence comprises a plurality of CpG Islands (CGI) methylated in the genome of extraembryonic ectoderm (ExE) and not methylated in corresponding epiblast or adult tissue, and determining a tissue of origin by calculating a relative abundance of haplotypes from the methylated genomic regions by defining a tissue-specific index (TSI) for each haplotype. In some embodiments, the TSI is calculated by the formula:

$$T S I = \frac{{\sum }_{j = 1}^{n} 1 - \frac{{10}^{PKR ( j )}}{{10}^{PKR ( max )}}}{n - 1}$$

wherein n is the number of tissues, PKR (j) is the fraction of a specific haplomer in tissue, and j and PKR max are PKR of the highest methylated tissue. In some embodiments, the sequences comprise one or more sequences provided in Table 2.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. These and other changes can be made to the disclosure in light of the detailed description.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

All patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or prior publication, or for any other reason. All statements as to the date or representation as to the contents of these documents is based on the information available to the applicants and does not constitute any admission as to the correctness of the dates or contents of these documents.

One skilled in the art readily appreciates that the present invention is well adapted to carry out the objects and obtain the ends and advantages mentioned, as well as those inherent therein. The details of the description and the examples herein are representative of certain embodiments, are exemplary, and are not intended as limitations on the scope of the invention. Modifications therein and other uses will occur to those skilled in the art. These modifications are encompassed within the spirit of the invention. It will be readily apparent to a person skilled in the art that varying substitutions and modifications may be made to the invention disclosed herein without departing from the scope and spirit of the invention.

The articles “a” and “an” as used herein in the specification and in the claims, unless clearly indicated to the contrary, should be understood to include the plural referents. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process. Furthermore, it is to be understood that the invention provides all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. It is contemplated that all embodiments described herein are applicable to all different aspects of the invention where appropriate. It is also contemplated that any of the embodiments or aspects can be freely combined with one or more other such embodiments or aspects whenever appropriate. Where elements are presented as lists, e.g., in Markush group or similar format, it is to be understood that each subgroup of the elements is also disclosed, and any element(s) can be removed from the group. It should be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not in every case been specifically set forth in so many words herein. It should also be understood that any embodiment or aspect of the invention can be explicitly excluded from the claims, regardless of whether the specific exclusion is recited in the specification. For example, any one or more active agents, additives, ingredients, optional agents, types of organism, disorders, subjects, or combinations thereof, can be excluded.

Where the claims or description relate to a composition of matter, it is to be understood that methods of making or using the composition of matter according to any of the methods disclosed herein, and methods of using the composition of matter for any of the purposes disclosed herein are aspects of the invention, unless otherwise indicated or unless it would be evident to one of ordinary skill in the art that a contradiction or inconsistency would arise. Where the claims or description relate to a method, e.g., it is to be understood that methods of making compositions useful for performing the method, and products produced according to the method, are aspects of the invention, unless otherwise indicated or unless it would be evident to one of ordinary skill in the art that a contradiction or inconsistency would arise.

Where ranges are given herein, the invention includes embodiments in which the endpoints are included, embodiments in which both endpoints are excluded, and embodiments in which one endpoint is included and the other is excluded. It should be assumed that both endpoints are included unless indicated otherwise. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise. It is also understood that where a series of numerical values is stated herein, the invention includes embodiments that relate analogously to any intervening value or range defined by any two values in the series, and that the lowest value may be taken as a minimum and the greatest value may be taken as a maximum. Numerical values, as used herein, include values expressed as percentages. For any embodiment of the invention in which a numerical value is prefaced by “about” or “approximately”, the invention includes an embodiment in which the exact value is recited. For any embodiment of the invention in which a numerical value is not prefaced by “about” or “approximately”, the invention includes an embodiment in which the value is prefaced by “about” or “approximately”.

“Approximately” or “about” generally includes numbers that fall within a range of 1% or in some embodiments within a range of 5% of a number or in some embodiments within a range of 10% of a number in either direction (greater than or less than the number) unless otherwise stated or otherwise evident from the context (except where such number would impermissibly exceed 100% of a possible value). It should be understood that, unless clearly indicated to the contrary, in any methods claimed herein that include more than one act, the order of the acts of the method is not necessarily limited to the order in which the acts of the method are recited, but the invention includes embodiments in which the order is so limited. It should also be understood that unless otherwise indicated or evident from the context, any product or composition described herein may be considered “isolated”.

### EXAMPLES

### Introduction

Recently, a new generation of biomarkers have been established with the discovery of genetic alterations that are responsible for the initiation and progression of human cancers. These alterations include single-base substitutions, insertions, deletions and translocations. These somatic mutations can also be detected in cell-free circulating tumor DNA (cfDNA) [6]. The development of non-invasive liquid biopsy methods based on the analysis of ctDNA provides an opportunity for a new generation of diagnostic approaches. A recently developed blood test was able to detect eight common cancer types through the assessment of the levels of circulating proteins and mutations in cfDNA, with a sensitivity ranging from 69-98% and specificity higher than 99% [7]. However, mutation-based liquid biopsy tests suffer from low sensitivity due to intra- and inter-tumor heterogeneity [8] since not all samples of one cancer type contain the same genetic driver alterations. For instance, analysis of lung adenocarcinoma samples has led to the identification of 22 drivers [9] but up to 25% of patients contain no genetic alterations in any of those genes [10, 11]. Furthermore, the existence of low frequency sub-clones renders mutation-based diagnostics even more complicated: in stage I disease, the fraction of cfDNA is around 0.1% and thus, detection of sub-clonal mutations with a frequency of 5% in early stage disease challenges the detection limit of current sequencing technologies [13].

In recent years, DNA methylation profiling has been adopted as a promising approach for liquid biopsies [14]. Aberrant DNA methylation is ubiquitous in human cancer and has been shown to occur early during carcinogenesis, thus providing attractive potential biomarkers for the early detection of cancer [15]. Compared to a normal genome, cancer genomes are globally hypomethylated and locally hypermethylated in CpG Islands (CGI) [16, 17]. Markers associated with these two features have been extensively used for methylation-based ctDNA detection [18, 19]. For instance, FBN1, FBN2, HLTF, PHACTR3, SEPT9, SNCA, SST, TAC1, VIM have been used individually for colorectal cancer (CRC) detection [20]. However, single gene-based diagnosis suffers from low accuracy due to tumor heterogeneity. Genome-wide assays such as whole-genome bisulfite sequencing (WGBS) and reduced representation bisulfite sequencing (RRBS) thus have been tested to improve prediction performance. For instance, plasma hypomethylation gave a sensitivity and specificity of 74% and 94%, respectively, for the detection of nonmetastatic cancer cases, when a mean of 93 million WGBS reads per case were obtained [18]. Recently, methylated DNA immunoprecipitation sequencing (MeDIP-seq), a genome-wide assay, was demonstrated for sensitive tumour detection and classification using plasma cell-free DNA methylomes [21]. In terms of analytical methods, since CpG mean methylation-based methods are not sufficiently sensitive for early cancer detection, methylation haplotype blocks (MHB; i.e. co-methylated stretches of DNA) have been used instead and are able to detect 2% tumor DNA [22]. This approach has led to the development of a novel methylation haplotype analysis tool, CancerDetector, which is able to detect 0.1% tumor DNA as demonstrated by spike-in experiments [23]. Genome-wide assays are promising in terms of both sensitive early cancer detection and cancer type classification, but in general suffer from higher cost and longer turnaround-time. Targeted assays which only interrogate a set of predefined genomic regions represent a solution that balances information obtain and cost. For instance, padlock-based targeted sequencing have been evaluated for noninvasive detection of hepatocellular carcinoma (HCC) with a sensitivity of 83.3% and specificity of 90.5% using as few as 10 markers [25]. Detection HCC is relatively easy compared to other cancer types since up to 20% of cfDNA derives from liver tissue even in normal controls [26]. Recently, a marker with 4 consecutive CpG sites were characterized with amplicon-based bisulfite sequencing in breast cancer and a fully methylated pattern was identified for early identification of metastasis [27]. Though with sensitivity as low as 25%, this method represents a novel way for joint analysis of multiple CpG sites in a single locus. The published studies that use targeted sequencing were mainly to address the detection of single cancer type, thus ultrasensitive methods for non-invasive detection of multiple cancer types remain to be developed. Epigenetic restriction of extraembryonic lineages mirrors the somatic transition to cancer [28]. An extraembryonic methylation signature was discovered to distinguish cancer samples from matched normal tissues for almost all cancer types tested. Based on these findings, the extraembryonic signature, coupled with DNA methylation haplotype analysis, represents a universal framework for ultra-sensitive non-invasive early cancer diagnosis.

### Results

### Extraembryonically Hyper-Methylated CGIs Provide a Universal Cancer Signature

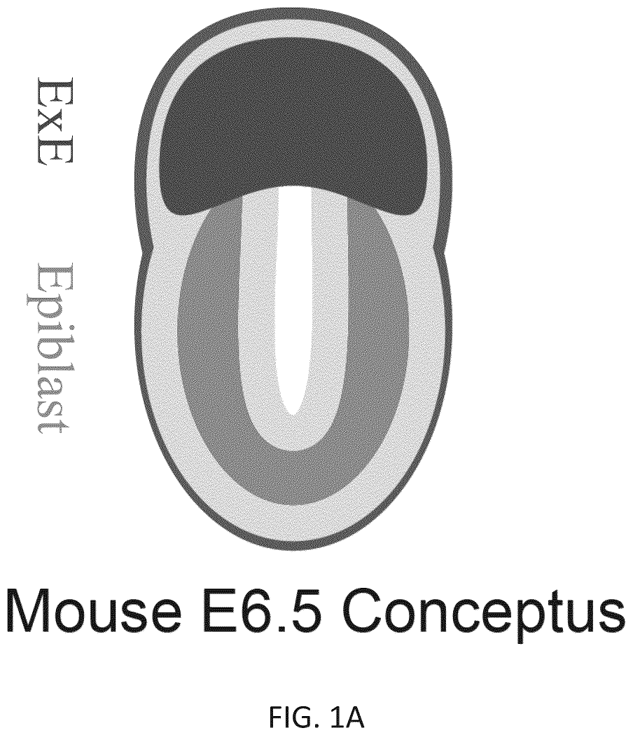

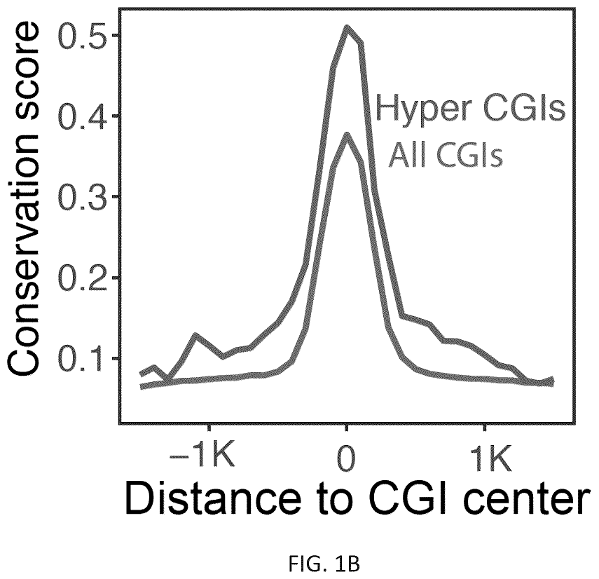

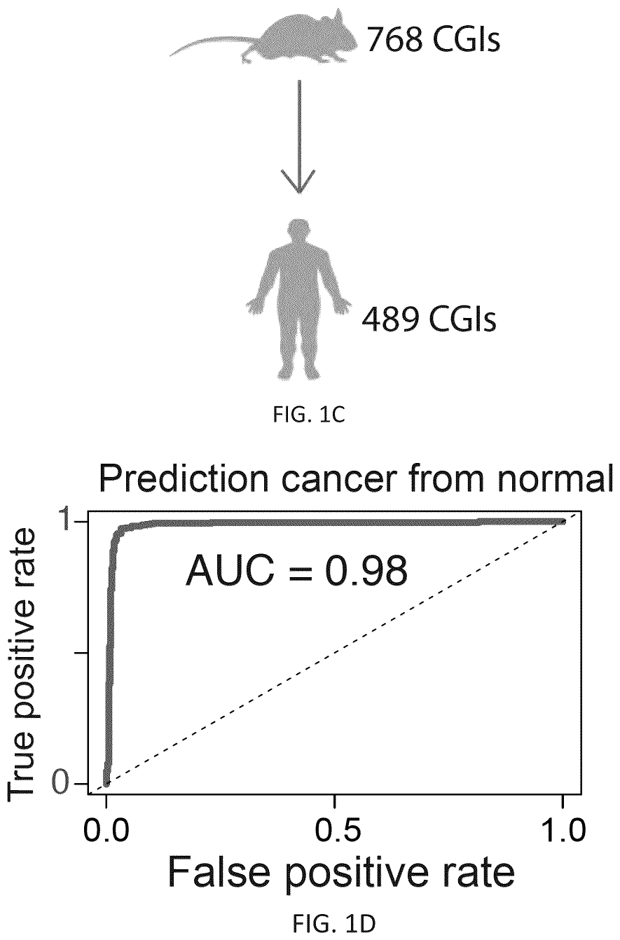

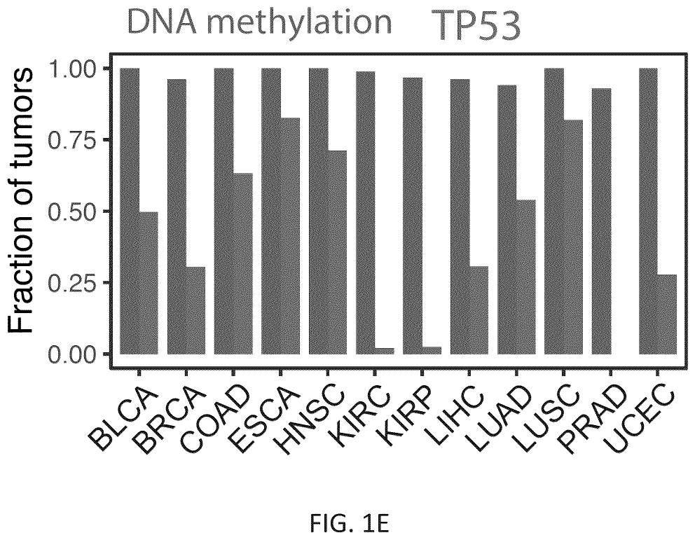

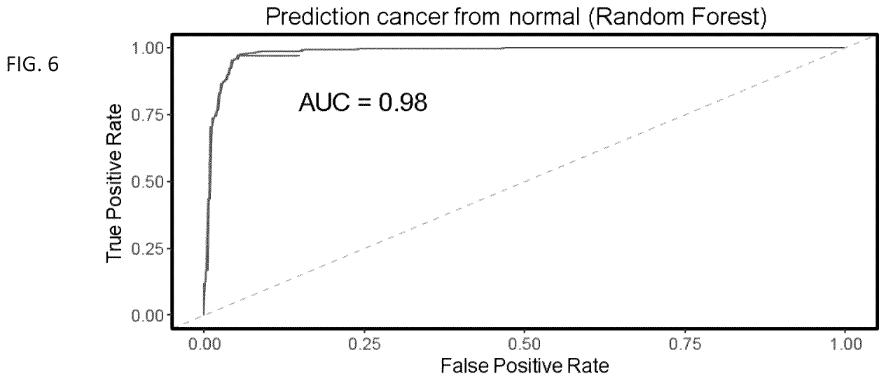

Placenta has long been considered to be a tissue of pseudo-malignancy [29], with several phenotypes, such as its angiogenic, immune suppressive and invasive abilities, reminiscent of human cancer. The DNA methylation landscape of extraembryonic ectoderm (ExE), the progenitor of placenta, was compared with that of the epiblast of a mouse E6.5 conceptus (FIG. 1A). Using this data, ExE hyper-methylated CGIs (ExE Hyper CGIs) was identified as a DNA methylation signature able to distinguish these two tissue types. Interestingly, ExE Hyper CGIs are more conserved on the sequence level than the genomic background (FIG. 1B), and the majority of ExE Hyper CGIs in mouse have a human ortholog that is localized near a CGI (FIG. 1C). Strikingly, it was found that the ExE Hyper CGI signature is hypermethylated in 14 cancer type profiled within The Cancer Genome Atlas (TCGA) project that contain matched normal tissues [28]. The only exception is thyroid cancer, which could potential be explained by the observation that FGF and WNT pathways are shared during tissue specification of ExE and normal thyroid epithelia [30]. Performance of ExE hyper CGIs was next tested in cancer prediction using TCGA pan-cancer data sets. When TCGA samples were randomly assigned into a training and validation set, ExE hyper CGIs were able to classify tumor versus normal samples with high sensitivity and specificity using support vector machine (SVM) classification method (Methods, AUC=0.98, FIG. 1D). Similar result was obtained when an independent method random forest was applied on the same data sets (AUC=0.98, Methods and FIG. 6). This observation suggests that a vast majority of cases of each tumor type can be correctly identified when using ExE hyper CGIs, and that human cancer types are significantly more homogenous when analyzed for their methylation status of ExE hyper CGIs than when profiled for the mutation status of any driver genes (FIG. 1E). For example, although somatic mutations in TP53 represent the most frequent genetic alterations in human cancer, many cancer types such as kidney renal papillary cell carcinoma (KIRP) and kidney renal clear cell carcinoma (KIRC) demonstrate low mutation frequencies in TP53 (FIG. 1E). ExE hyper CGIs thus represent a novel DNA methylation signature for pan-cancer diagnosis and the basis for developing the instant non-invasive liquid biopsy platform.f.

### DNA Methylation Haplotypes Improve Detection Sensitivity

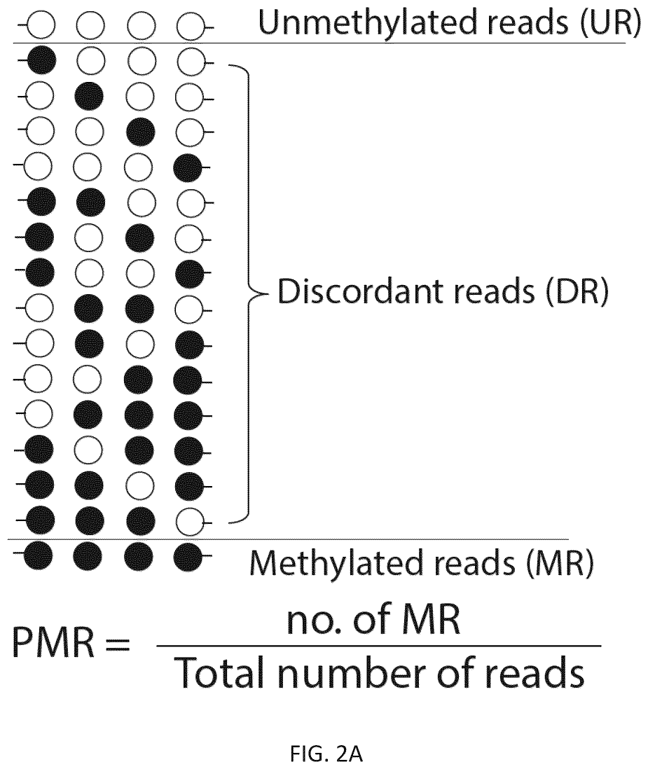

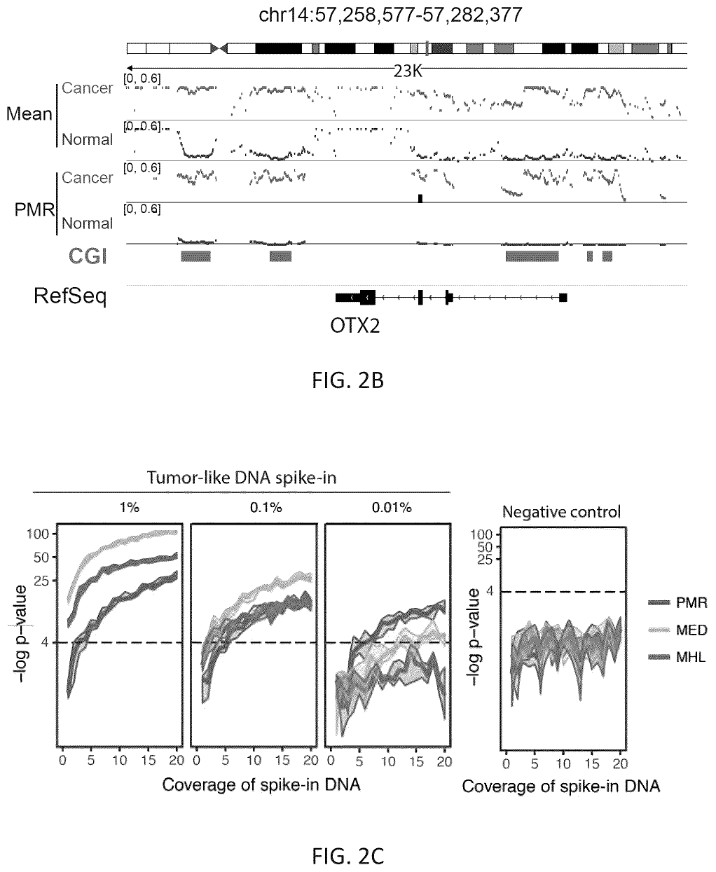

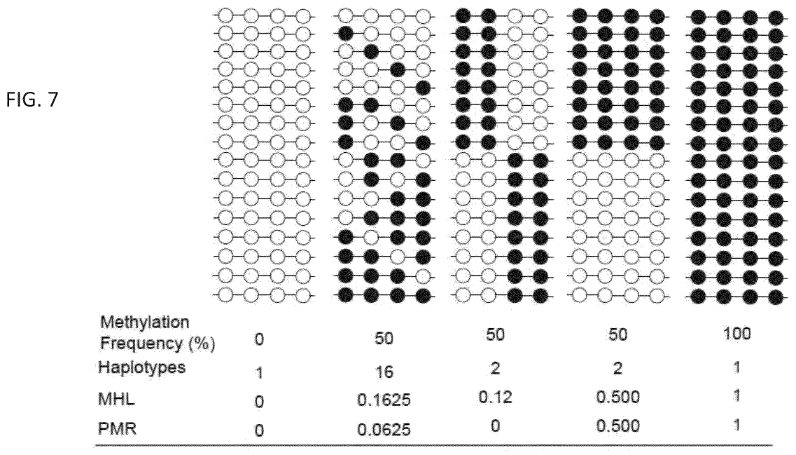

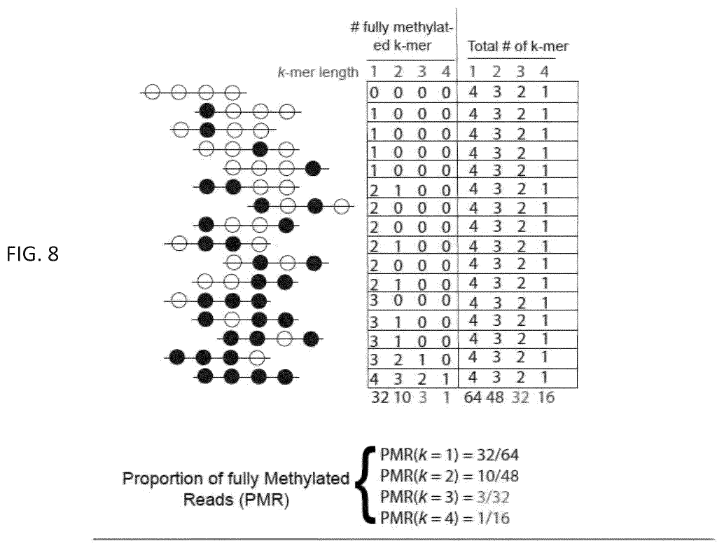

The development of non-invasive liquid biopsy methods based on the DNA methylation of ctDNA has revolutionized cancer diagnosis [21]; however, several challenges remain. First, disordered methylation is frequently observed in cancer [31], which is one of the reasons why single CpG-based diagnostic platforms suffer from low sensitivity. The overall sensitivity of SEPT9, for example, is only 60% for colorectal cancer (CRC) detection [32]. Second, the fraction of ctDNA among cell-free DNA is as low as 0.01% in early stage diseases [33], which requires nearly zero background contributed by normal cells to make tumor cell detection possible. However, normal cells acquire low-level methylation (˜1%) when measured at single CpG sites due to noise, aging and other stochastic processes [35]. To overcome these issues, a novel approach was developed based on the observation that DNA methylation haplotypes, measured in phase on the same molecule, provide a better choice for diagnostic purposes. Even when measured from bulk data, DNA methylation information obtained from a single sequenced fragment is guaranteed to stem from a single chromosome and a single cell. Thus, the methylation pattern of CpGs of each fragment represents a discrete DNA methylation haplotype (FIG. 2A). In normal somatic tissues, fully methylated reads are very rare when analyzing ExE Hyper CGIs. Thus, the proportion of fully methylated reads (PMR) calculated from sequencing data represents a novel way to quantify the extent of DNA methylation (FIGS. 7 and 8). This approach significantly reduces background noise as compared to standard approaches. For example, OTX2 is a developmental regulator and hypermethylated in ExE and placenta, and also serves as one of the ExE hyper CGI markers. When its mean methylation level was used, a considerable extent of background noise was observed in normal samples. In contrast, PMR-based quantification at this locus significantly reduced background noise (FIG. 2B).

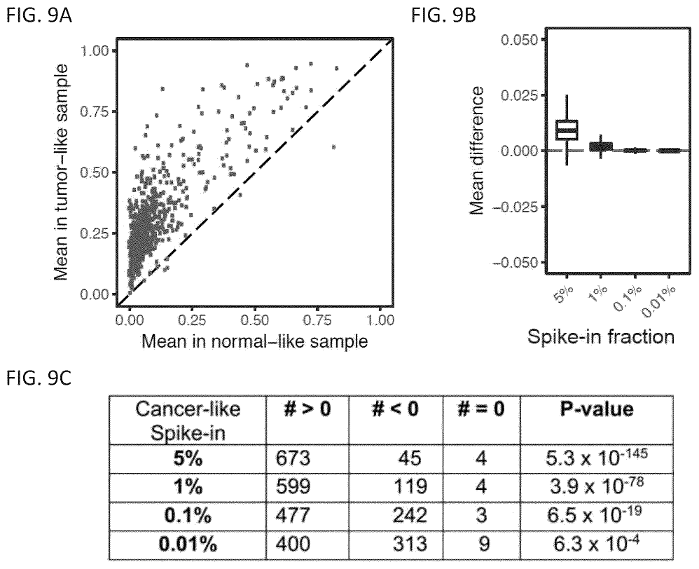

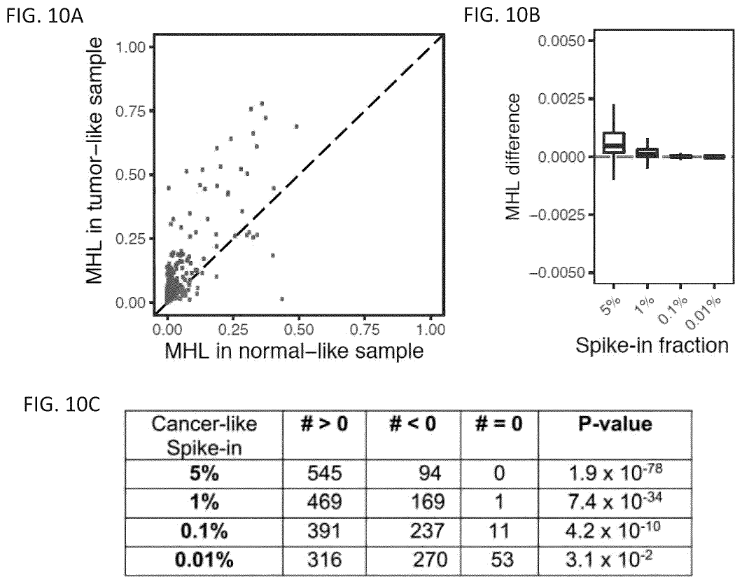

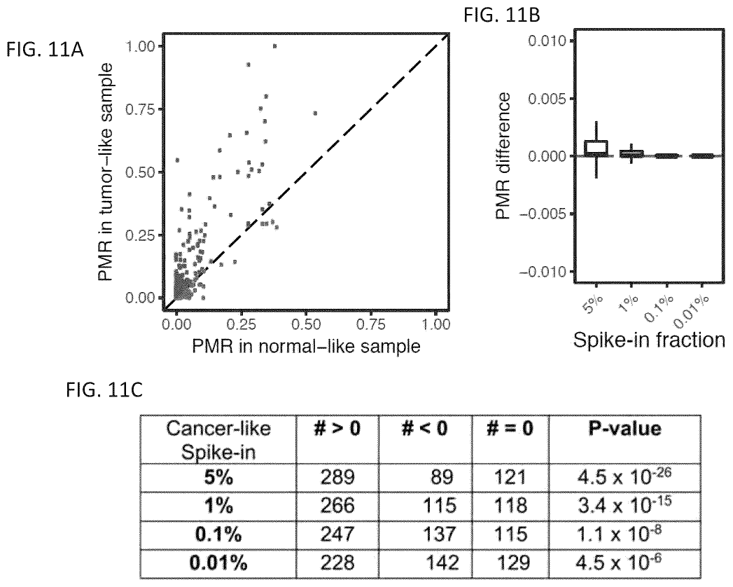

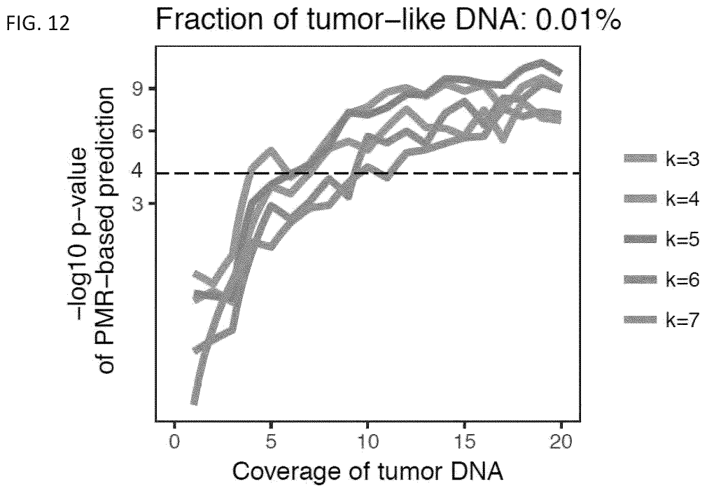

To evaluate the performance of PMR, silico simulations were performed by randomly sampling sequencing reads from normal-like tissue epiblast as well as tumor-like tissue ExE as a spike-in. The fraction of spike-in ranged from 0.01% to 1%, which matches the fraction of ctDNA in cell-free DNA (Methods). Besides mean methylation and PMR, DNA methylation haplotype load (MHL), which quantifies level of co-methylation [22], was also included for comparison (FIGS. 9, 10 and 11). Using this approach, all three methods had significant predictive power in both the 1% and 0.1% spike-in groups; however, when the fraction of spike-in decreased to 0.01%, only the PMR-based prediction reached significance when the mean coverage of the spike-in was 5× or higher (FIG. 2C). It is noted that PMR is a k-mer-based approach, and highest sensitivity was achieved with a k of 5 when it was tested on simulated 0.01% spike-in group (FIG. 12).

### An Efficient Workflow to Enrich for DNA Methylation Haplotypes

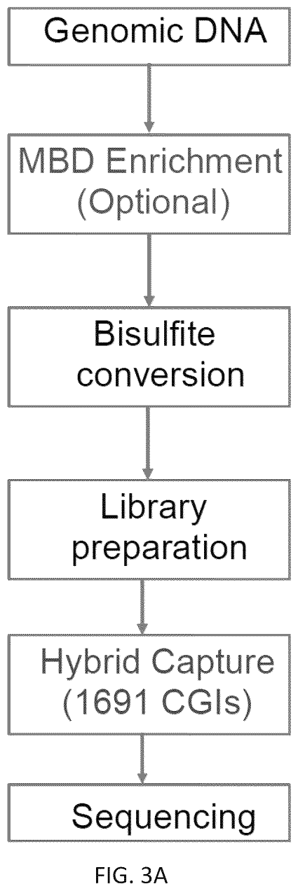

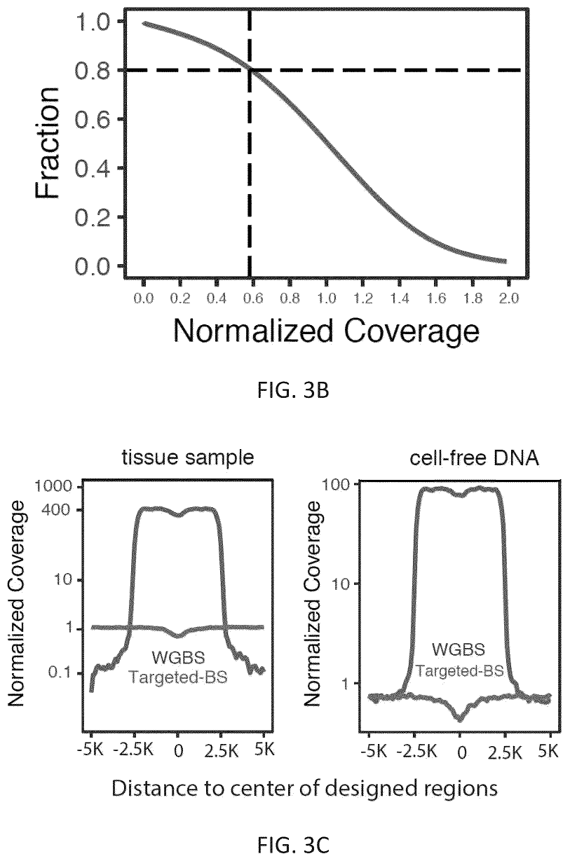

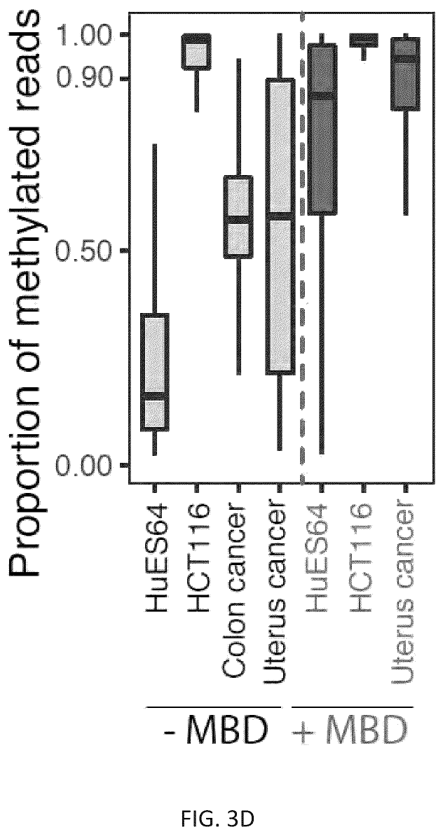

Several recent studies have adopted either reduced-representation bisulfite sequencing (RRBS) [22], whole-genome bisulfite sequencing (WGBS) or methylated DNA immunoprecipitation sequencing (MeDIP-seq) approaches to profile cell-free DNA, all of which suffer from poor coverage in regions of interest in exchange for the availability of genome-wide information. Instead of these approaches, targeted bisulfite sequencing (targeted-BS) were used since this assay produces data with a stronger signal from regions of interest, associated with a lower cost as compared to the other methods. To this end, a highly specific target-capture pipeline was established using the SeqCap Epi technology [36], which is able to enrich ExE hyper CGIs (6.2 Mb in total; Methods) with an on-target rate of ˜80%. Given the low fraction of tumor-derived DNA in plasma, most sequencing reads obtained from plasma samples stem from normal DNA, which is largely unmethylated in the target regions. Methylated DNA fragments were further specifically enriched using the MBD2 protein, followed by targeted-BS, to analyze tumor-derived DNA (FIG. 3A). The customized probe set exhibits similar performance as commercial probe sets in terms of enrichment uniformity; specifically, 80% of loci have a coverage higher than 60% of the median coverage (FIG. 3B). When tested on biopsy samples of both tumor and normal tissues, the targeted-BS approach achieved>400 fold enrichment compared to WGBS; even on challenging samples such as cell-free DNA, >100 fold enrichment was observed (FIG. 3C). When coupling this workflow with MBD enrichment before bisulfite conversion, high specificity was achieved with on average more than 90% of reads partially or fully methylated (FIG. 3D).

### Unbiased Measurement of DNA Methylation Across Assays

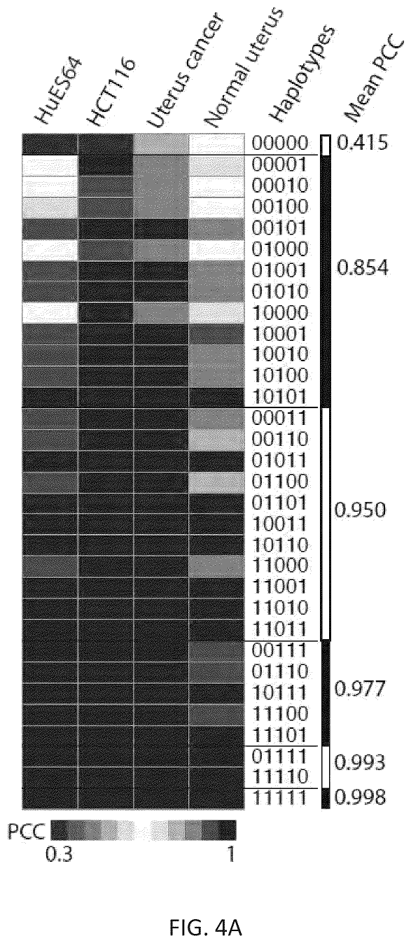

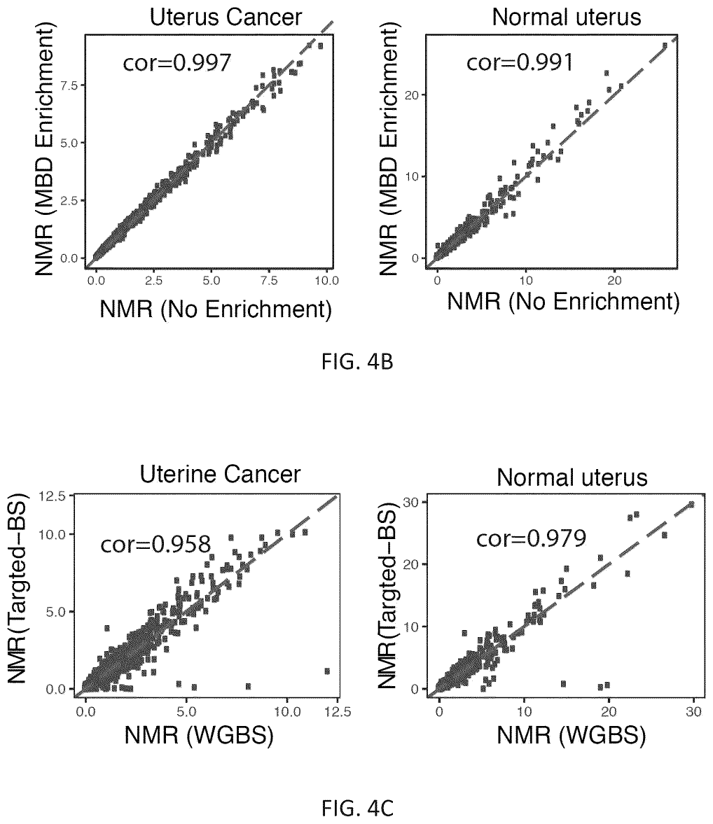

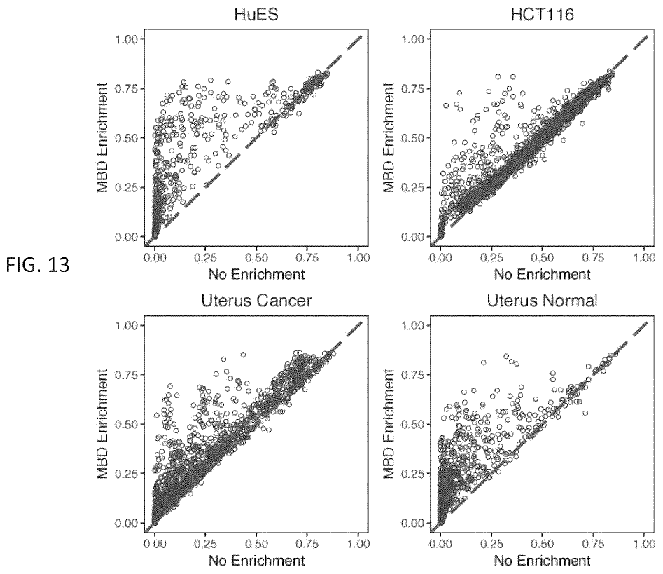

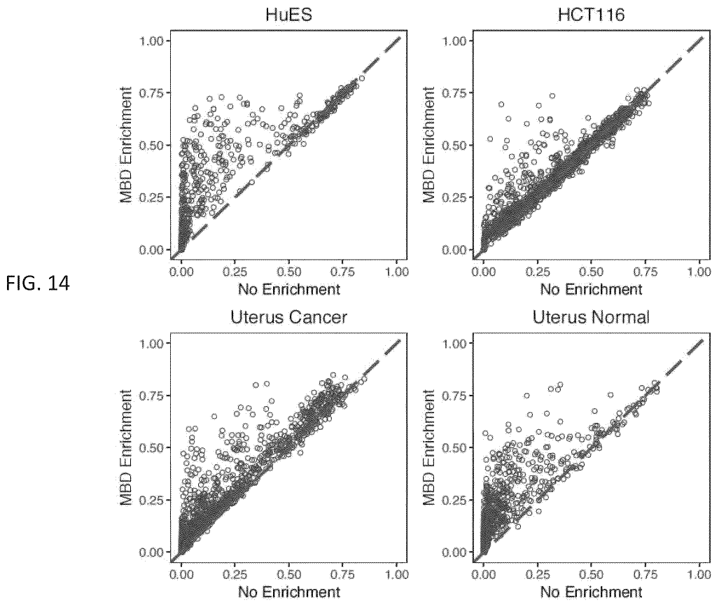

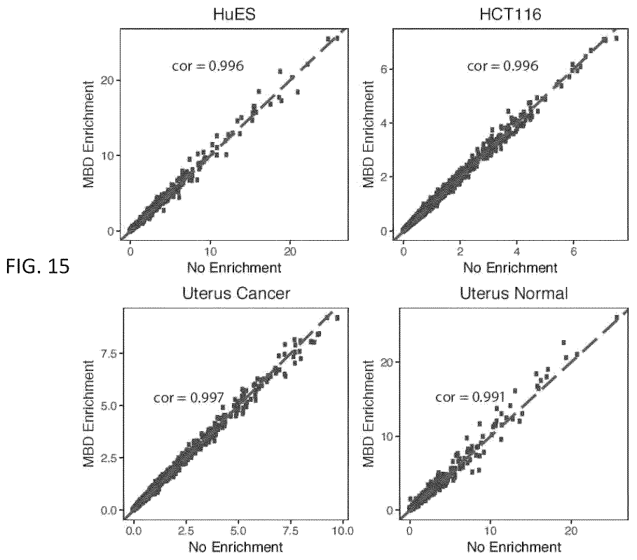

By definition, PMR is the number of fully methylated k-mer haplotypes divided by the total number of k-mers in each genomic feature such as a CpG island, where it was set to 5 to maximize sensitivity (FIG. 12). Similarly, MHL is the normalized PMR at different k-mer lengths (Methods, k=1 to 10). Thus both PMR and MHL are haplotype-based methods that are locally normalized, but neither of them can be applied without bias across assays: when the same sample was profiled by targeted-BS with or without MBD enrichment, neither PMR nor MHL were comparable between these two assays (FIGS. 13 and 14). An alternative method of global normalization normalizes the number of haplotypes in a region by total number of haplotypes across all regions. For a given haplotype width k (i.e. k=5), the globally normalized coverage of each type of DNA methylation haplotype was compared for the same samples that were profiled by both assays—with or without MBD enrichment. Two cell lines (HuES64 and HCT116) and two primary tissues (normal uterus and uterine cancer) were profiled using this approach. The highest Pearson correlation coefficient (PCC) was observed between these two approaches when using the number of fully methylated DNA methylation haplotypes (mean PCC=0.998) (FIG. 4A). For example, when the normalized coverage of fully methylated reads (NMR) was assessed for normal uterus and uterine cancer, nearly perfect correlation was observed between assays with or without MBD enrichment (PCC>0.99, p-value<10⁻¹⁶) (FIG. 4B and FIG. 15). As expected, unbiased measurements were also observed when comparing targeted-BS and WGBS, though there were larger variations due to lower sequencing depth in WGBS-assayed samples (PCC=0.958 for uterine cancer and PCC=0.979 for normal uterus, p-value<10⁻¹⁶) (FIG. 4C). Taking together, NMR is an unbiased metric to quantify haplotype-level DNA methylation across WGBS and targeted-BS approaches with or without MBD enrichment. This methodological improvement led to the development of markers from existing data and validate them on new data.

### Ultra-Sensitive Cancer Detection Using DNA Methylation Haplotypes

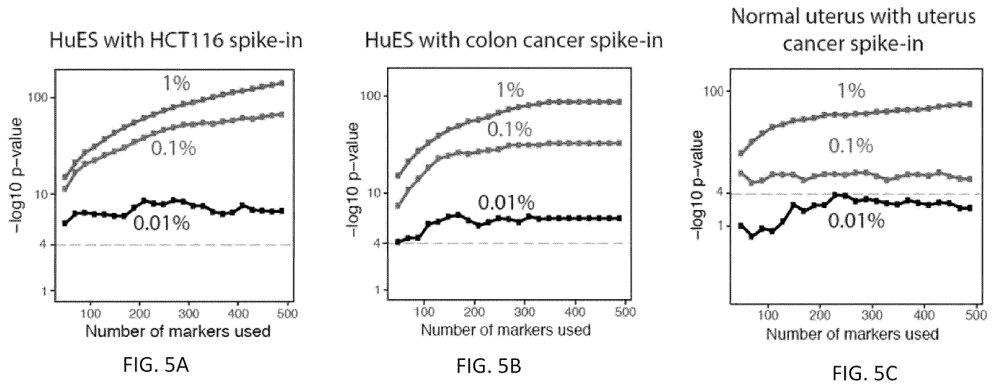

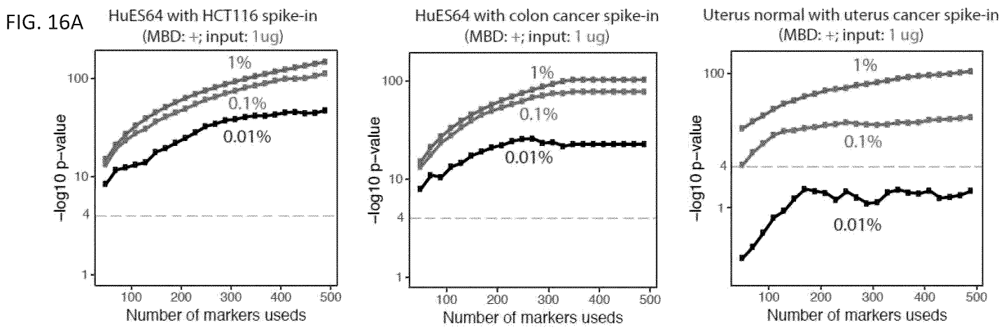

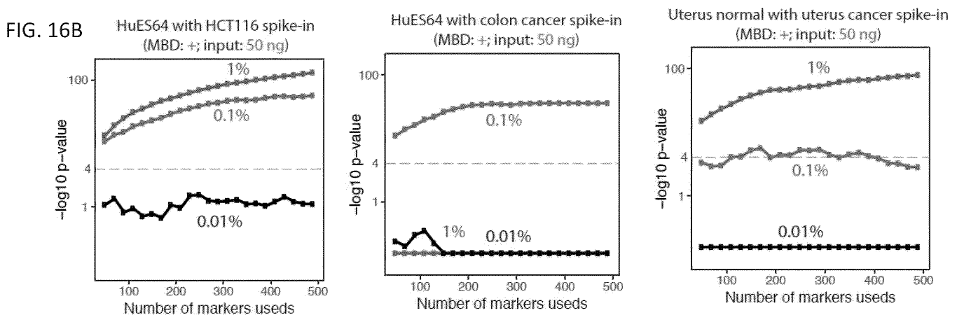

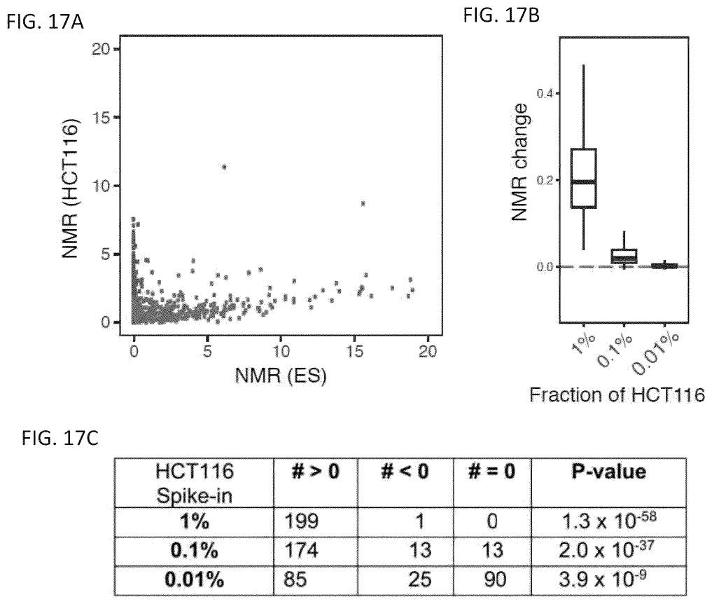

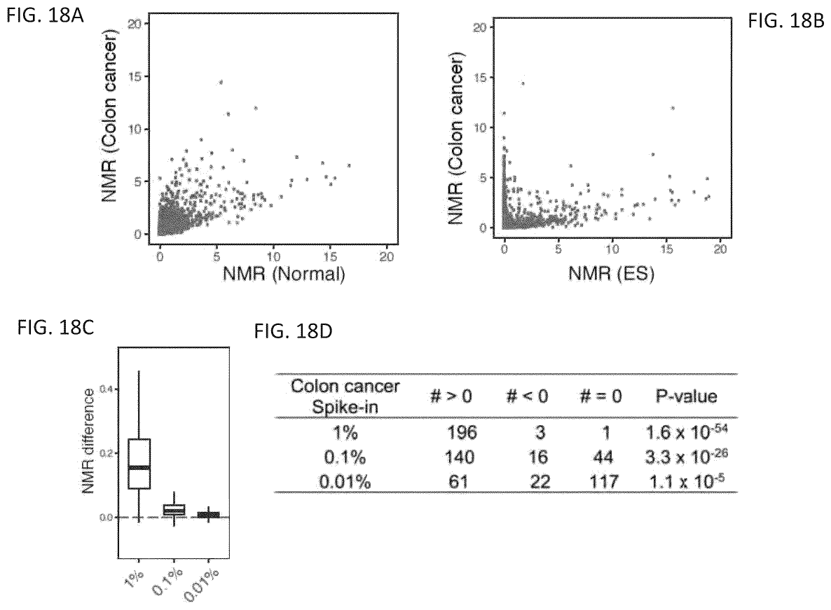

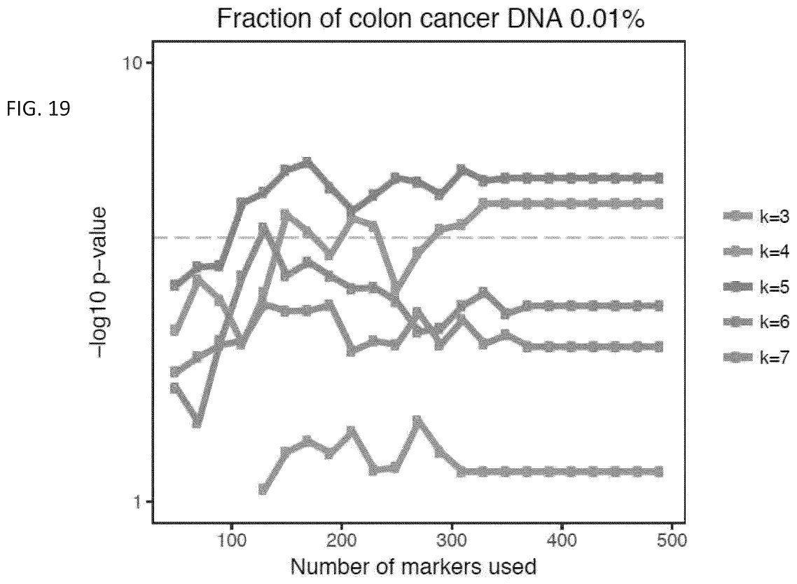

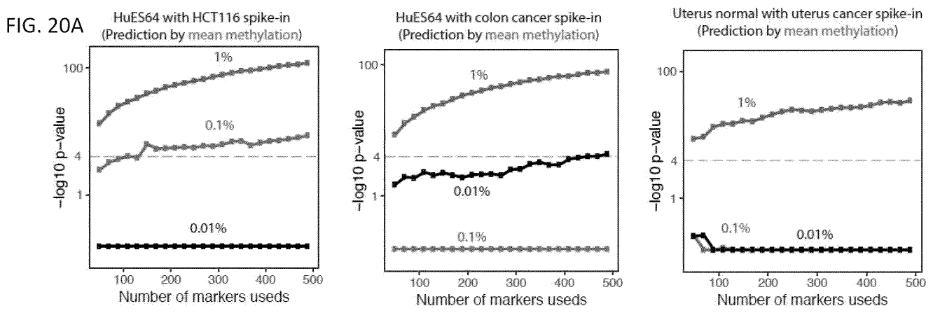

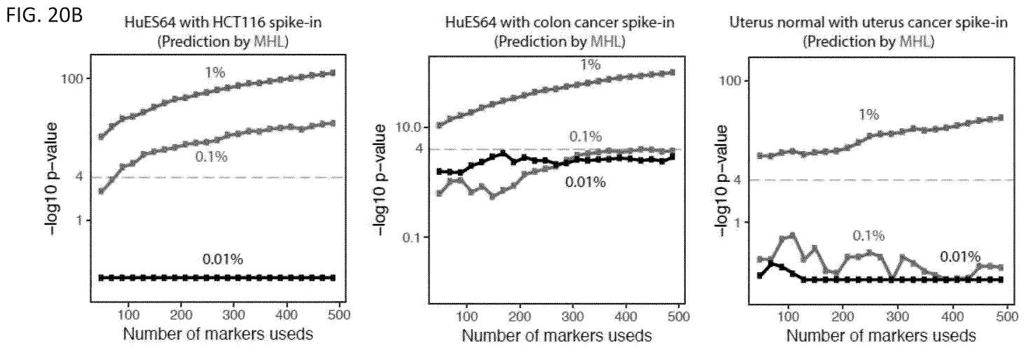

Since ctDNA levels are very low in most early-stage and many advanced stage cancer patients [6], a major challenge is how to identify a trace amount of cfDNAs out of total cfDNAs. To test the sensitivity of the MBD enrichment-based workflow, experiments mixing DNA from ES cells (HuES64) were first performed with DNA from a colon cancer cell line, HCT116, as spike-in. The NMR-based method confidently predicted 0.01% spike-in when at least 1 μg of total input DNA was used (FIG. 16A). However, when 50 ng of total input DNA was analyzed, the prediction limit dropped to 0.1% (FIG. 16B). Novel analytical approaches such as NMR could improve sensitivity on targeted-BS data even without MBD enrichment which performs well with lower input DNA. When testing the targeted-BS workflow without MBD enrichment with 50 ng DNA as input, conditions with 0.01% spike-in were correctly identified with as few as 50 CGIs (FIG. 5A and FIG. 17). In contrast, mean methylation and MHL-based methods were only able to correctly identify the tumor signature when the fraction of spike-in DNA was larger than 0.1% (FIG. 20A). Detection of HCT116 DNA is easier than that of other samples, since its genome is almost fully methylated, next performed were similar dilution experiments with primary colon cancer tissue as the spike-in. Again, NMR-based method confidently detected cancer DNA spiked-in at 0.01% (FIGS. 5B and 18), while mean methylation and MHL-based methods only detected 1% cancer DNA spiked-in (FIG. 20B). Note that the detection sensitivity depends on the background noise stemming from normal cells; for example, when uterine cancer DNA was spiked-in with normal uterus DNA, the NMR-based method was able to detect 0.1% cancer DNA (FIG. 5C), while both mean methylation and MHL-based methods only detected 1% cancer DNA (FIG. 20C). Detection sensitivity also depends on choices of parameters; for example, highest sensitivity was achieved when k-mer length was set to 5 for NMR method (FIG. 19).

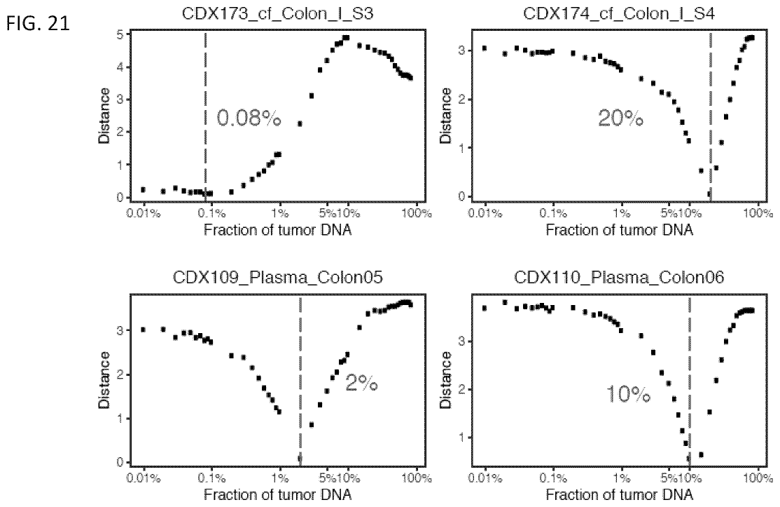

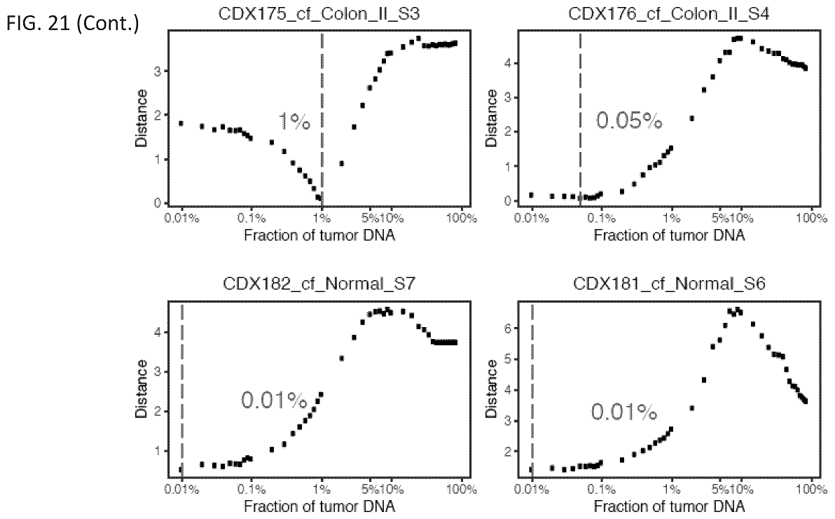

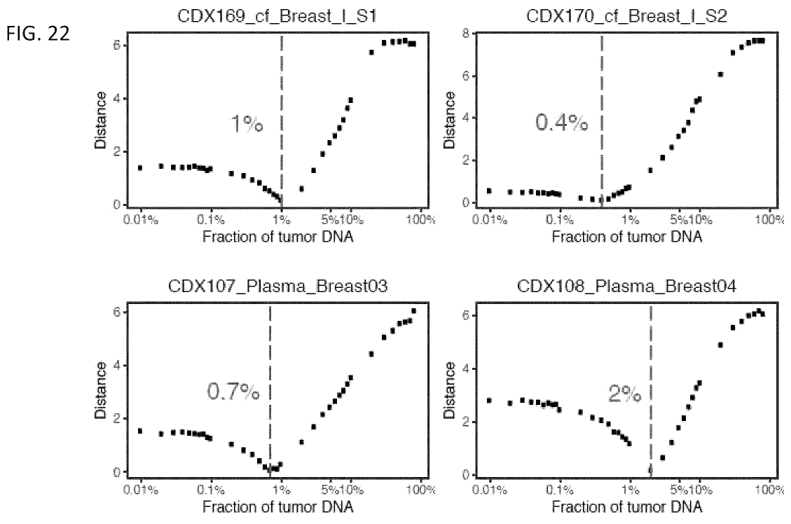

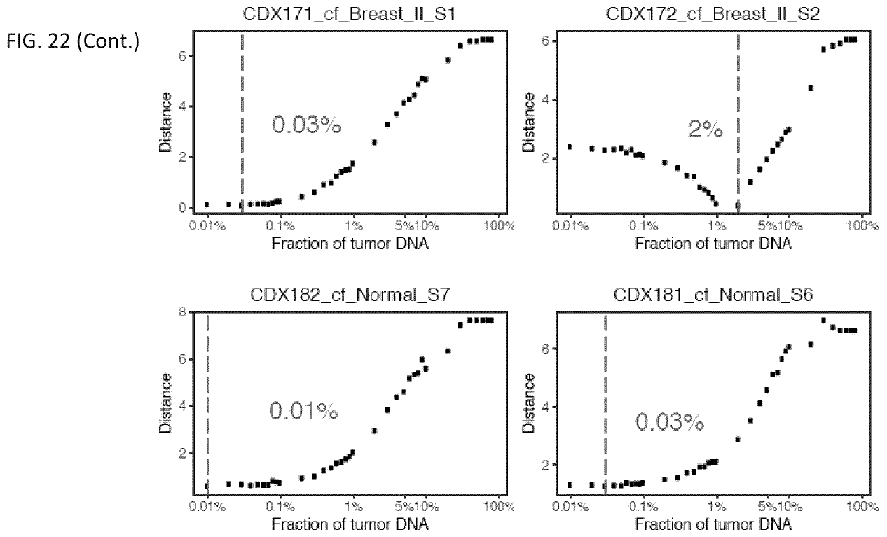

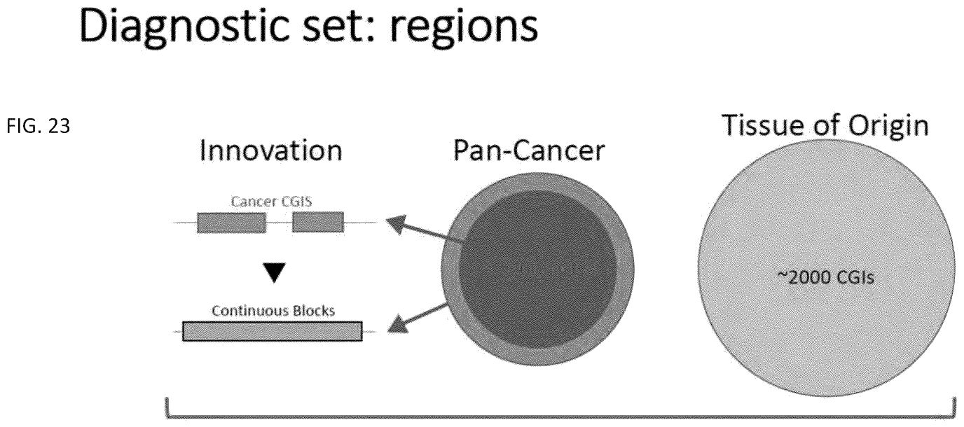

Finally, the experimental and computational pipeline on plasma samples obtained from patients with colon adenocarcinoma were tested using age-matched normal individuals as negative controls. Included were two samples each from stage I, II and III patients, respectively. The platform was capable of detecting all cancers, including those in stage I, with high confidence (FDR<1%), and no false positives were observed (Table 1A). To further assess the sensitivity of the method, the fraction of reads predicted to stem from tumor cells were estimated. In the colon cancer cohort, the estimated fraction of cancer DNA ranged from 0.05% to 20% (Methods; FIG. 21), suggesting a prediction resolution of 0.05% for colon cancer. A breast cancer patient cohort (Infiltrating ductal carcinoma) was next tested, including two cases each for stages I, II and III. The NMR-based method detected 5 of 6 cancer samples, with one false negative of a stage II sample, CDX171 (FDR<1%, Table 1B), while mean methylation and MHL-based methods correctly identified only one sample each. The false negative can likely be attributed to a low tumor DNA fraction, as the estimated tumor fraction for CDX171 was around 0.03%, which is similar to background noise (Methods and FIG. 22).

### Machine Learning Methods

Extensive prediction models using machine learning approaches (random forest, support vector machines, and deep learning) was developed to estimate the full probability distribution of all haplotypes across ExE sites with regard to each tumor type. These methods will improve the prediction accuracy of the cell type of origin based on cfDNA samples.

### Pan-Cancer Associated Methylation Sites are Provided in Table 3

### Discussion

DNA methylation haplotypes have been used for many years, but only recently was shown to be useful for cancer diagnosis; for instance, Guo et. al. demonstrated that a DNA methylation haplotype-based metric, MHL, combined with methylation haplotype blocks (MHB). An experimental and computational framework for ultra-sensitive, non-invasive early cancer detection using fully methylated DNA methylation haplotypes was proposed. As demonstrated by dilution experiments, this framework outperformed mean methylation and MHL-based methods and was able to detect 0.01% colon cancer spike-in with as few as 50 CGIs. When tested on human plasma samples, both colon and breast cancer samples were correctly detected at early stages, with a detection limit of 0.05%; this threshold is sufficiently sensitive to detect most stage I tumors. This is the first study that utilizes a universal cancer signature for non-invasive pan-cancer diagnosis, which is potentially cost effective compared to genome-wide assays [21].

### Cohort

As described below, tumor and normal samples from 12 cancer types, with the exception of bladder and prostate cancer, in which only normal samples were included. For cancer types, different major subtypes were included whenever possible, featured by breast invasive carcinoma. All samples were processed uniformly in Broad Institute and profiled by targeted bisulfite sequencing with customized probe design that covers 8M of genomic regions which are mainly hyper-methylated in human cancer.

| Tissue | Status | Subtype | # Samples |
|---|---|---|---|
| AML | Tumor | | 8 |
| Bladder | Tumor | | 0 |
| | Normal | | 2 |
| Breast | Tumor | breast (TN) | 8 |
| | | breast PR − ER − her2+ | 3 |
| | | breast PR − ER + her2− | 1 |
| | | breast PR − ER + her2+ | 3 |
| | | breast PR + ER − her2− | 1 |
| | | breast PR + ER − her2+ | 2 |
| | | breast PR + ER + her2− | 8 |
| | | breast PR + ER + her2+ | 6 |
| | Normal | | 4 |
| Colon | Tumor | Colon adenocarcinoma | 10 |
| | Normal | | 2 |
| Esophagus | Tumor | Esophageal carcinoma | 10 |
| | Normal | | 2 |
| Kidney | Tumor | Kidney carcinoma | 14 |
| | | (renal clear cell, renal | |
| | | papillary cell) | |
| | Normal | | 2 |
| Liver | Tumor | Liver hepatocellular | 0 |
| | | carcinoma | |
| | Normal | | 2 |
| Lung | Tumor | adenocarcinoma | 13 |
| | | squamous | 6 |
| | Normal | | 2 |
| Ovary | Tumor | Adenocarcinoma of | 15 |
| | | ovary | |
| | Normal | | 2 |
| Pancreas | Tumor | Pancreatic | 11 |
| | | adenocarcinoma | |
| | Normal | | 2 |
| Prostate | Tumor | | 0 |
| | Normal | | 2 |
| Stomach | Tumor | Adenocarcinoma of | 5 |
| | | stomach | |
| | Normal | | 2 |

### Tissue of Origin

An ultra-sensitive method was developed based on DNA methylation haplotypes of extraembryonically methylated CpG islands. This method could detect 0.05% of tumor DNA from cell-free DNA of patient plasma. To further develop this method and predict tissue of origin with high sensitivity, the method includes identifying cancer specific DNA methylation haplotypes. For each CpG position in designed regions, the relative abundance of all possible k-mer haplotypes (k=5) were calculated across all tissue samples, which includes tumor and normal samples. Then a tissue-specific index (TSI) was defined for each k-mer as:

$$T S I = \frac{{\sum }_{j = 1}^{n} 1 - \frac{{10}^{PKR ( j )}}{{10}^{PKR ( max )}}}{n - 1}$$

Where n indicate the number tissues, PKR (j) denotes fraction of a specific k-mer in tissue j and PKR max denotes PKR of the highest methylation tissue. Cancer specific DNA methylation haplotypes were selected by TSI with a cutoff of 0.6. The addition of cancer-specific DNA methylation haplotypes to the original signature enables the prediction of tissue of origin with high sensitivity.

Identified regions of cancer-specific DNA methylation are provided in Table 2.

### Methods

### Targeted-BS and MBD Enrichment

Genomic DNA from cultured cells was extracted using Genomic DNA Clean & Concentrator kit (Zymo Research). Human tumor DNA was purchased from OriGene Technologies or BioChain Institute. Genomic DNA was sheared to average fragment size of 180-220 bp in 130 μl microTUBE using S2 focused-ultrasonicator (Covaris) for 300 sec at intensity 5, duty cycle 10 and 200 cycles per burst. The sheared DNA was concentrated with 1.8 volumes of Agencourt AMPure XP beads (Beckman Coulter) prior to bisulfite conversion. Purified human cell-free DNA and frozen human plasma from cancer patients were obtained from the BioChain Institute. Free circulating DNA was isolated from 4 ml human plasma using QIAamp MinElute ccfDNA Mini Kit (Qiagen) scaling up the reactions as described in manufacturer's manual. In order to enrich for methylated DNA, selected samples were processed with MethylMiner Methylated DNA Enrichment Kit (Thermo Fisher Scientific). DNA bound to MBD2 protein coupled to streptavidin beads was eluted with provided high-salt buffer in a single elution step and DNA was ethanol-precipitated. Pellets were dissolved in 20 μl water. Sheared genomic DNA, cfDNA and MBD-enriched DNA was bisulfite-converted using EpiTect Fast bisulfite conversion kit (Qiagen) following kit's instructions and extending the two 60° C. cycles to 20 min. Illumina library construction was performed post-bisulfite conversion using Accel-NGS Methyl-Seq kit (Swift Biosciences) following the manufacturer's recommendations for NimbleGen SepCap Epi Hybridization Capture (Appendix Section A). Libraries were amplified by 8-14 cycles of PCR using Accel-NGS Methyl-Seq Unique Dual Indexing primers (Swift Biosciences). SeqCap Epi hybridization reactions contained a total of 1 μg of a pool of 3-4 PCR-amplified pre-capture libraries, 2 μl of xGen Universal BlockersTS Mix (Integrated DNA Technologies) blocking oligonucleotides, and the custom SeqCap probe pool. After hybridization at 47° C. (typically ˜70 h), streptavidin pull-down and washes, the entire bead-bound captured material was amplified by 9-10 cycles of PCR. Hybrid-selected libraries were sequenced on an Illumina HiSeq 2500 instrument in rapid mode together with a 10% spike-in of a non-indexed PhiX174 library.

### Probe Set Design for Targeted-BS

1,265 CGIs were selected which are hypermethylated in extraembryonic tissues for targeted bisulfite-sequencing. Specifically, 473 CGIs are hypermethylated in mouse extraembryonic ectoderm and were lifted over to human genome; the rest is hypermethylated in 8 out of 14 TCGA cancer types and also human placenta. To cover loci with multiple hypermethylated CGIs, such as the OTX2 locus, CGIs that are 20k bp apart were merged. The resulting regions were extended 2k upstream and downstream, respectively, to cover CpG shores. Probes were designed by NimbleDesign with default parameters (design.nimblegen.com). The resulting design covers 6.1 Mbps with an estimated coverage of 98.2%.

### Data Processing

Raw sequencing reads were pre-processed by ‘trim_galore (v0.4.4)’, with the following parameters: ‘--clip_R1 5--three_prime_clip_R1 2--clip_R2 10--three_prime_clip_R2 2’. Low-quality base calls and adapters were trimmed off from the 3′ end of the reads by default. Trimmed reads were aligned to human reference genome GRCh37 using Bismark (v0.19.0) with default parameters. Duplicate reads were identified and removed using tools in Bismark. DNA methylation haplotypes were extracted using an in-house tool called mHaplotype (github.com/JiantaoShi/mHaplotype). Reads with methylated cytosines in a non-CpG context (CHG, CHH) were removed to eliminate potential bias caused by incomplete bisulfite conversion.

### In Silico Simulation

ExE and Epiblast represent typical tumor-like and normal-like genomes, respectively, in terms of DNA methylation landscapes. To evaluate the performance of different cancer prediction methods, in silico simulations were performed by randomly sampling sequencing reads from ExE and epiblast samples. Briefly, ExE and epiblast RRBS data were obtained from the public data set GSE98963, which contains 4 biological replicates for each tissue. DNA methylation haplotypes were extracted by the in-house tool ‘mHaplotype’ and biological replicates were pooled. Sequencing reads were randomly sampled from epiblast as well as ExE as spike-in, representing 1%, 0.1% and 0.01% of total reads, in three groups of simulations, respectively. In each group, the mean coverages of spike-in DNA ranged from 1 to 20, each with 10 replicates. Negative controls were also included, in which spike-in reads were sampled from epiblast.

### Estimating Methylation Levels

Mean methylation levels were estimated as the number of sites reporting a C, divided by the total number of sites reporting a C or T. The methylation pattern of CpGs on each fragment represents a discrete DNA methylation haplotype. Methylation haplotype load (MHL), the normalized fraction of methylated haplotypes at different lengths, was calculate as previously described [22]:

$$M H L = \frac{{\sum }_{k = 1}^{10} {w}_{k} * {PMR}_{k}}{{\sum }_{k = 1}^{10} {w}_{k}}$$

$${w}_{k} = k$$

Where k is the length of haplotypes, and for a haplotype of length L, all substrings with length from 1 to a maximum of 10 in this calculation was considered. wₖ is the weight for k-mer haplotype. In the present study, wₖ=k was applied. PMRₖ is the fraction of fully successive methylated CpGs for haplotypes of length k (k-mer) (FIG. 8). In this study, k was set to 5 to maximize detection sensitivity (FIG. 12). To calculate the normalized coverage of fully methylated reads (NMR), the number of fully methylated k-mers was determined in each CGI which is then divided by the total number of fully methylated k-mers in all designed regions, followed by a mean scaling. Again, k was set to 5 to maximize detection sensitivity (FIG. 19).

### Prediction the Presence of Cancer DNA

Presence of cancer-specific DNA methylation suggests presence of cancer DNA in a mixture. As described above, four metrics, mean methylation, MHL, PMR and NMR, were used for DNA methylation quantification and cancer prediction. Four types of samples were used for prediction: tumor tissue samples, normal tissue samples, normal cfDNA samples and patient cfDNA samples. For a given CGI, the DNA methylation in these groups were represented as Me₍ₜ₎, Me₍ₙ₎, Me_{(f)}, Me₍ₚ₎, respectively. Regardless of metrics used, the general steps for cancer prediction are quite similar.

### Marker Identification

ExE hyper CGIs are largely hyper-methylated in cancer vs. normal. Markers were redefined for each cancer type and metric used to maximize detection sensitivity. Specifically, tumor tissue samples were compared to normal tissue samples to define markers that are hypermethylated in tumors with a threshold of 0.1 (Me₍ₜ₎-Me₍ₙ₎>0.1).

### Marker Refinement

Selected markers were then ranked in descending order based on the difference of methylation between tumor samples and normal cfDNA (Me(t)-Me(f)). The top 200 regions were chosen as markers for cancer prediction.

### Significance Test

The test samples were compared to normal cfDNA samples using cancer markers defined above, the resulting difference of methylation was defined as ΔMe=Me₍ₚ₎-Me_{(f)}. Instead of using actual values of methylation difference, the number of markers with increased methylation (ΔMe>0) and decreased methylation (ΔMe<0) were counted. The higher the number of markers with increased methylation, the more likely a cancer sample is detected. P-value is computed by one-sided binomial tested and corrected for multiple testing using Benjamini-Hochberg procedure.

### Predicting the Fraction of Tumor DNA

The fraction of tumor DNA was predicted by comparing the observed data to simulated normal cfDNA data with tumor DNA as spike-in, the fractions of which ranged from 0.01% to 100%. NMR was compared between observed (NMPₛ) and simulated samples (NMPₛ) using pre-defined markers for each cancer type, the resulting difference was denoted as ΔNMR=NMRs−NMRₒ. Then a distance metric was calculated as follows:

$$d = abs ( log 2 ( \frac{sum ( Δ NMR > 0 )}{sum ( Δ NMR < 0 )} ) )$$

The predicted tumor fraction was defined as the value that minimized the distance d.

### Cancer Prediction Using TCGA 450K Array Data

In order to evaluate performance of ExE hyper CGIs in cancer prediction, 14 TCGA cancer types were tested that contain matched normal tissues in TCGA. Samples from thyroid cancer data set were removed, since thyroid cancer and normal thyroid tissue cannot be distinguished by ExE hyper CGIs [28]. This pan-cancer cohort consists of 685 tumor samples and 710 normal samples.

Half of the samples were randomly chosen as a training set, and the remainder were used for validation. Support vector machine (SVM) with a Gaussian kernel from the R package kernlab was used for classification. To resolve dependence between ExE hyper CGIs, 50 CGIs were randomly chosen for classification and this process was repeated 200 times, the resulting prediction scores were averaged as final concensus scores. Receiver operating characteristic (ROC) curves were generated by R package ROCR.

Similarly, random forest (RF) was implemented using the ‘randomForest’ function of the ‘randomForest’ R package, using default parameter settings. Classification accuracy was calculated as the proportion of samples in the validation set that the trained model correctly classified. False positive rate and true positive rate were calculated using the ‘roc’ function of the ‘PROC’ R package, based on the ‘out-of-bag’ votes for the training data. Area under the ROC curve (AUC) was calculated based on these values using the ‘auc’ function, also from the ‘PROC’ package.

### Data Availability

All datasets have been deposited in the Gene Expression Omnibus and are accessible under GSE84236. Additional data include: TCGA DNA methylation, mutation data, and the full name of tumor types from the Broad Firehose (gdac.broadinstitute.org).

| Disease Name | Cohort | Cases |
|---|---|---|
| Adrenocortical carcinoma | ACC | 92 |
| Bladder urothelial carcinoma | BLCA | 412 |
| Breast invasive carcinoma | BRCA | 1098 |
| Cervical and endocervical cancers | CESC | 307 |
| Cholangiocarcinoma | CHOL | 51 |
| Colon adenocarcinoma | COAD | 460 |
| Colorectal adenocarcinoma | COADREAD | 631 |
| Lymphoid Neoplasm Diffuse | DLBC | 58 |
| Large B-cell Lymphoma | | |
| Esophageal carcinoma | ESCA | 185 |
| FFPE Pilot Phase II | FPPP | 38 |
| Glioblastoma multiforme | GBM | 613 |
| Glioma | GBMLGG | 1129 |
| Head and Neck squamous | HNSC | 528 |
| cell carcinoma | | |
| Kidney Chromophobe | KICH | 113 |
| Pan-kidney cohort | KIPAN | 973 |
| (KICH + KIRC + KIRP) | | |
| Kidney renal clear cell carcinoma | KIRC | 537 |
| Kidney renal papillary | KIRP | 323 |
| cell carcinoma | | |
| Acute Myeloid Leukemia | LAML | 200 |
| Brain Lower Grade Glioma | LGG | 516 |
| Liver hepatocellular carcinoma | LIHC | 377 |
| Lung adenocarcinoma | LUAD | 585 |
| Lung squamous cell carcinoma | LUSC | 504 |
| Mesothelioma | MESO | 87 |
| Ovarian serous | OV | 602 |
| cystadenocarcinoma | | |
| Pancreatic adenocarcinoma | PAAD | 185 |
| Pheochromocytoma and | PCPG | 179 |
| Paraganglioma | | |
| Prostate adenocarcinoma | PRAD | 499 |
| Rectum adenocarcinoma | READ | 171 |
| Sarcoma | SARC | 261 |
| Skin Cutaneous Melanoma | SKCM | 470 |
| Stomach adenocarcinoma | STAD | 443 |
| Stomach and Esophageal carcinoma | STES | 628 |
| Testicular Germ Cell Tumors | TGCT | 150 |
| Thyroid carcinoma | THCA | 503 |
| Thymoma | THYM | 124 |
| Uterine Corpus Endometrial | UCEC | 560 |
| Carcinoma | | |
| Uterine Carcinosarcoma | UCS | 57 |
| Uveal Melanoma | UVM | 80 |

### REFERENCES

1. McGranahan, N. and C. Swanton, Clonal Heterogeneity and Tumor Evolution: Past, Present, and the Future. Cell, 2017. 168 (4): p. 613-628.
2. Winawer, S. J., et al., Prevention of colorectal cancer by colonoscopic polypectomy. The National Polyp Study Workgroup. N Engl J Med, 1993. 329 (27): p. 1977-81.
3. Karam, A. K. and B. Y. Karlan, Ovarian cancer: the duplicity of CA125 measurement. Nat Rev Clin Oncol, 2010. 7 (6): p. 335-9.
4. Gao, Y., et al., Evaluation of Serum CEA, CA19-9, CA72-4, CA125 and Ferritin as Diagnostic Markers and Factors of Clinical Parameters for Colorectal Cancer. Sci Rep, 2018. 8(1): p. 2732.
5. Nordstrom, T., et al., Prostate-specific antigen (PSA) density in the diagnostic algorithm of prostate cancer. Prostate Cancer Prostatic Dis, 2018. 21 (1): p. 57-63.
6. Bettegowda, C., et al., Detection of circulating tumor DNA in early- and late-stage human malignancies. Sci Transl Med, 2014. 6 (224): p. 224ra24.
7. Cohen, J. D., et al., Detection and localization of surgically resectable cancers with a multi-analyte blood test. Science, 2018.
8. Yates, L. R. and P. J. Campbell, Evolution of the cancer genome. Nat Rev Genet, 2012. 13(11): p. 795-806.
9. Lawrence, M. S., et al., Discovery and saturation analysis of cancer genes across 21 tumour types. Nature, 2014. 505 (7484): p. 495-501.
10. Pao, W. and K. E. Hutchinson, Chipping away at the lung cancer genome. Nat Med, 2012. 18 (3): p. 349-51.
11. Cancer Genome Atlas Research, N., Comprehensive molecular profiling of lung adenocarcinoma. Nature, 2014. 511 (7511): p. 543-50.
12. Phallen, J., et al., Direct detection of early-stage cancers using circulating tumor DNA. Sci Transl Med, 2017. 9 (403).
13. Corcoran, R. B. and B. A. Chabner, Application of Cell-free DNA Analysis to Cancer Treatment. N Engl J Med, 2018. 379 (18): p. 1754-1765.
14. Laird, P. W., The power and the promise of DNA methylation markers. Nat Rev Cancer, 2003. 3 (4): p. 253-66.
15. Baylin, S. B., et al., Aberrant patterns of DNA methylation, chromatin formation and gene expression in cancer. Hum Mol Genet, 2001. 10 (7): p. 687-92.
16. Berman, B. P., et al., Regions of focal DNA hypermethylation and long-range hypomethylation in colorectal cancer coincide with nuclear lamina-associated domains. Nat Genet, 2011. 44 (1): p. 40-6.
17. Zhou, W., et al., DNA methylation loss in late-replicating domains is linked to mitotic cell division. Nat Genet, 2018. 50 (4): p. 591-602.
18. Chan, K. C., et al., Noninvasive detection of cancer-associated genome-wide hypomethylation and copy number aberrations by plasma DNA bisulfite sequencing. Proc Natl Acad Sci USA, 2013. 110 (47): p. 18761-8.
19. Kang, S., et al., Cancer Locator: non-invasive cancer diagnosis and tissue-of-origin prediction using methylation profiles of cell-free DNA. Genome Biol, 2017. 18 (1): p. 53.
20. Leygo, C., et al., DNA Methylation as a Noninvasive Epigenetic Biomarker for the Detection of Cancer. Dis Markers, 2017. 2017: p. 3726595.
21. Shen, S. Y., et al., Sensitive tumour detection and classification using plasma cell-free DNA methylomes. Nature, 2018.
22. Guo, S., et al., Identification of methylation haplotype blocks aids in deconvolution of heterogeneous tissue samples and tumor tissue-of-origin mapping from plasma DNA. Nat Genet, 2017. 49 (4): p. 635-642.
23. Li, W., et al., CancerDetector: ultrasensitive and non-invasive cancer detection at the resolution of individual reads using cell-free DNA methylation sequencing data. Nucleic Acids Res, 2018.
24. Diep, D., et al., Library-free methylation sequencing with bisulfite padlock probes. Nat Methods, 2012. 9 (3): p. 270-2.
25. Xu, R. H., et al., Circulating tumour DNA methylation markers for diagnosis and prognosis of hepatocellular carcinoma. Nat Mater, 2017. 16 (11): p. 1155-1161.
26. Sun, K., et al., Plasma DNA tissue mapping by genome-wide methylation sequencing for noninvasive prenatal, cancer, and transplantation assessments. Proc Natl Acad Sci USA, 2015. 112 (40): p. E5503-12.
27. Widschwendter, M., et al., Methylation patterns in serum DNA for early identification of disseminated breast cancer. Genome Med, 2017. 9 (1): p. 115.
28. Smith, Z. D., et al., Epigenetic restriction of extraembryonic lineages mirrors the somatic transition to cancer. Nature, 2017. 549 (7673): p. 543-547.
29. Novakovic, B. and R. Saffery, Placental pseudo-malignancy from a DNA methylation perspective: unanswered questions and future directions. Front Genet, 2013. 4: p. 285.
30. Kurmann, A. A., et al., Regeneration of Thyroid Function by Transplantation of Differentiated Pluripotent Stem Cells. Cell Stem Cell, 2015. 17 (5): p. 527-42.
31. Landau, D. A., et al., Locally disordered methylation forms the basis of intratumor methylome variation in chronic lymphocytic leukemia. Cancer Cell, 2014. 26 (6): p. 813-825.
32. Nian, J., et al., Diagnostic Accuracy of Methylated SEPT9 for Blood-based Colorectal Cancer Detection: A Systematic Review and Meta-Analysis. Clin Transl Gastroenterol, 2017. 8(1): p. e216.
33. Aravanis, A. M., M. Lee, and R. D. Klausner, Next-Generation Sequencing of Circulating Tumor DNA for Early Cancer Detection. Cell, 2017. 168 (4): p. 571-574.
34. Gentilini, D., et al., Stochastic epigenetic mutations (DNA methylation) increase exponentially in human aging and correlate with X chromosome inactivation skewing in females. Aging (Albany NY), 2015. 7 (8): p. 568-78.
35. Wahlberg, P., et al., DNA methylome analysis of acute lymphoblastic leukemia cells reveals stochastic de novo DNA methylation in CpG islands. Epigenomics, 2016. 8 (10): p. 1367-1387.
36. Li, Q., et al., Post-conversion targeted capture of modified cytosines in mammalian and plant genomes. Nucleic Acids Res, 2015. 43 (12): p. e81.
37. Krueger, F. and S. R. Andrews, Bismark: a flexible aligner and methylation caller for Bisulfite-Seq applications. Bioinformatics, 2011. 27 (11): p. 1571-2.

TABLE 1A

| | | | # > | # < | # = | | |
|---|---|---|---|---|---|---|---|
| ID | Stages | Fraction | 0 | 0 | 0 | P-value | FDR |
| CDX173 | I | 0.08% | 124 | 61 | 15 | 2.11E−06 | 8.42E−06 |
| CDX174 | I | 20% | 190 | 10 | 0 | 1.47E−44 | 1.03E−43 |
| CDX175 | II | 1% | 162 | 31 | 7 | 6.65E−23 | 3.33E−22 |
| CDX176 | II | 0.05% | 117 | 74 | 9 | 1.14E−03 | 3.43E−03 |
| CDX109 | III | 2% | 176 | 22 | 2 | 2.53E−31 | 1.52E−30 |
| CDX110 | III | 10% | 192 | 8 | 0 | 3.58E−47 | 2.86E−46 |
| CDX182 | Normal | 0.01% | 85 | 102 | 13 | 9.06E−01 | 1.00E+00 |
| CDX181 | Normal | 0.01% | 63 | 120 | 17 | 1.00E+00 | 1.00E+00 |

TABLE 1B

| | | | # > | # < | # = | | |
|---|---|---|---|---|---|---|---|
| ID | Stages | Fraction | 0 | 0 | 0 | P-value | FDR |
| CDX169 | I | 1% | 150 | 45 | 5 | 1.07E−14 | 6.42E−14 |
| CDX170 | I | 0.4% | 122 | 72 | 6 | 2.03E−04 | 8.14E−04 |
| CDX171 | II | 0.03% | 77 | 119 | 4 | 9.99E−01 | 1.00E+00 |
| CDX172 | II | 2% | 162 | 36 | 2 | 1.38E−20 | 9.66E−20 |
| CDX107 | III | 0.7% | 125 | 72 | 3 | 9.75E−05 | 4.88E−04 |
| CDX108 | III | 2% | 169 | 29 | 2 | 1.57E−25 | 1.26E−24 |
| CDX182 | Normal | 0.01% | 75 | 116 | 9 | 9.99E−01 | 1.00E+00 |
| CDX181 | Normal | 0.03% | 65 | 127 | 8 | 1.00E+00 | 1.00E+00 |

TABLE 2
TOO Methylation Sites

| | | |
|---|---|---|
| chr1: 1072370-1072847 | chr11: 65190825-65191058 | chr16: 72821141-72821592 |
| chr1: 10895896-10896117 | chr11: 65222491-65222750 | chr16: 73099813-73100791 |
| chr1: 109203594-109204378 | chr11: 65341621-65342501 | chr16: 743925-745943 |
| chr1: 1093212-1093476 | chr11: 65343330-65343849 | chr16: 78079753-78080166 |
| chr1: 110185962-110186164 | chr11: 65553750-65555573 | chr16: 80574742-80575090 |
| chr1: 110626529-110627484 | chr11: 65779312-65779767 | chr16: 80965953-80966478 |
| chr1: 110880395-110880624 | chr11: 66034752-66035054 | chr16: 84029457-84029710 |
| chr1: 111505882-111507007 | chr11: 66035217-66035447 | chr16: 84328520-84328720 |
| chr1: 111746338-111747303 | chr11: 66049751-66050229 | chr16: 84346477-84346931 |
| chr1: 113044411-113044992 | chr11: 66314208-66314455 | chr16: 84401958-84402497 |
| chr1: 113392143-113392807 | chr11: 66335576-66336151 | chr16: 85171020-85171323 |
| chr1: 113497987-113498206 | chr11: 67232299-67232558 | chr16: 85783863-85785131 |
| chr1: 1141671-1142150 | chr11: 67770427-67771629 | chr16: 85863382-85863601 |
| chr1: 11538670-11540342 | chr11: 67806252-67806611 | chr16: 85932122-85932942 |
| chr1: 116694665-116694983 | chr11: 68611251-68611807 | chr16: 86546360-86546632 |
| chr1: 116710838-116711260 | chr11: 69258150-69258544 | chr16: 87902455-87903460 |
| chr1: 11710460-11710788 | chr11: 69924339-69925197 | chr16: 88292764-88293010 |
| chr1: 11779567-11780016 | chr11: 705795-706534 | chr16: 88716990-88717606 |
| chr1: 118727817-118728097 | chr11: 70962174-70964161 | chr16: 88803803-88804112 |
| chr1: 120835962-120839391 | chr11: 71954817-71955659 | chr16: 88850205-88850537 |
| chr1: 12655927-12656248 | chr11: 720562-721369 | chr16: 89070647-89070904 |
| chr1: 1362955-1363299 | chr11: 72301303-72301746 | chr16: 89267824-89268087 |
| chr1: 1370768-1371449 | chr11: 72463093-72463717 | chr16: 89268493-89268865 |
| chr1: 13839506-13840613 | chr11: 72492282-72492644 | chr16: 89323281-89323661 |
| chr1: 13909607-13909842 | chr11: 74022429-74022703 | chr16: 89632593-89632799 |
| chr1: 14026482-14027200 | chr11: 75236190-75237781 | chr16: 90014251-90014613 |
| chr1: 14219351-14219737 | chr11: 75917272-75917926 | chr17: 10632790-10633490 |
| chr1: 146556313-146556676 | chr11: 77122737-77123088 | chr17: 11501632-11502328 |
| chr1: 14924611-14925993 | chr11: 78673008-78673213 | chr17: 1163342-1163773 |
| chr1: 149605515-149605903 | chr11: 789872-790133 | chr17: 12692738-12693690 |
| chr1: 150254366-150254637 | chr11: 8102359-8102913 | chr17: 1390457-1390786 |
| chr1: 150266477-150266689 | chr11: 826942-827625 | chr17: 1395120-1395372 |
| chr1: 151300523-151300724 | chr11: 8284103-8285032 | chr17: 14212364-14212788 |
| chr1: 151445872-151446142 | chr11: 86382696-86383586 | chr17: 15244706-15245126 |
| chr1: 151693992-151694282 | chr11: 87908244-87908614 | chr17: 15466360-15466843 |
| chr1: 151812254-151812525 | chr11: 9025096-9026315 | chr17: 1546743-1547324 |
| chr1: 151966633-151966893 | chr11: 93583375-93583717 | chr17: 1551731-1553249 |
| chr1: 152079998-152081705 | chr11: 94473536-94474338 | chr17: 15847758-15849513 |
| chr1: 154298206-154298544 | chr11: 94501367-94502696 | chr17: 16283928-16284768 |
| chr1: 154732823-154733436 | chr11: 9634970-9636065 | chr17: 17685017-17687240 |
| chr1: 154971871-154972404 | chr11: 9779593-9780470 | chr17: 18965478-18965728 |
| chr1: 155043413-155043922 | chr11: 98891544-98891821 | chr17: 2627241-2628302 |
| chr1: 155830196-155830489 | chr12: 103350090-103350422 | chr17: 26578273-26578682 |
| chr1: 156051240-156051461 | chr12: 103351580-103352695 | chr17: 26645291-26645614 |
| chr1: 156616554-156616946 | chr12: 103359249-103359629 | chr17: 26698360-26699557 |
| chr1: 156646293-156647260 | chr12: 104850254-104852395 | chr17: 26711384-26712311 |
| chr1: 156814882-156815792 | chr12: 105478090-105478517 | chr17: 27038085-27038919 |
| chr1: 156893520-156894232 | chr12: 106532107-106533696 | chr17: 27332269-27333188 |
| chr1: 157963541-157963947 | chr12: 107711604-107714107 | chr17: 27503599-27504014 |
| chr1: 158119489-158119704 | chr12: 108297427-108297743 | chr17: 27942533-27945388 |
| chr1: 159141203-159141718 | chr12: 109162409-109162722 | chr17: 27949430-27950277 |
| chr1: 15929824-15930289 | chr12: 109729573-109729826 | chr17: 29298047-29298606 |
| chr1: 160040129-160040668 | chr12: 110150048-110150262 | chr17: 29718231-29719291 |
| chr1: 16085148-16085862 | chr12: 110156268-110156496 | chr17: 29814615-29815662 |
| chr1: 161228478-161229028 | chr12: 111471961-111473546 | chr17: 30593199-30594033 |
| chr1: 162760251-162760722 | chr12: 112204499-112204979 | chr17: 30845904-30846702 |
| chr1: 162792177-162792574 | chr12: 115104849-115105548 | chr17: 32953154-32953801 |
| chr1: 16543684-16544307 | chr12: 115120775-115122945 | chr17: 33787402-33787845 |
| chr1: 166134259-166136448 | chr12: 115135926-115136350 | chr17: 33814235-33814947 |
| chr1: 167789397-167789647 | chr12: 115889598-115889995 | chr17: 34091137-34091919 |
| chr1: 17033769-17034728 | chr12: 116354788-116355187 | chr17: 3438842-3439046 |
| chr1: 171810468-171811325 | chr12: 116946196-116946607 | chr17: 35060323-35060692 |
| chr1: 179555402-179555770 | chr12: 117316390-117317611 | chr17: 35303285-35303572 |
| chr1: 180881317-180882592 | chr12: 117536291-117537421 | chr17: 36102034-36104766 |
| chr1: 182584178-182584545 | chr12: 120031495-120033212 | chr17: 36105335-36105583 |
| chr1: 184633224-184633663 | chr12: 120799373-120799912 | chr17: 36575500-36575782 |
| chr1: 1875618-1875877 | chr12: 122277302-122277539 | chr17: 36584421-36585453 |
| chr1: 18971730-18972097 | chr12: 122667649-122668038 | chr17: 36728634-36729284 |
| chr1: 19970256-19971923 | chr12: 123380334-123380894 | chr17: 37365987-37366539 |
| chr1: 200860077-200860576 | chr12: 126018101-126018365 | chr17: 37856449-37856891 |
| chr1: 200992283-200992839 | chr12: 128850550-128850755 | chr17: 38020382-38020645 |
| chr1: 201368561-201369032 | chr12: 129787736-129788160 | chr17: 38347534-38347765 |
| chr1: 201450881-201451105 | chr12: 130526916-130527117 | chr17: 38497528-38498963 |
| chr1: 201475886-201476516 | chr12: 13152820-13153084 | chr17: 38501397-38501839 |
| chr1: 201708788-201709429 | chr12: 132312440-132315739 | chr17: 39683909-39684599 |
| chr1: 202936046-202936252 | chr12: 132689881-132690197 | chr17: 39705046-39705332 |
| chr1: 203456785-203457059 | chr12: 132690340-132690571 | chr17: 40250273-40250591 |
| chr1: 203598472-203598853 | chr12: 133463808-133464858 | chr17: 40332598-40333471 |
| chr1: 204159599-204159833 | chr12: 14927292-14928023 | chr17: 40440189-40441014 |
| chr1: 204797611-204797930 | chr12: 175667-176400 | chr17: 40805675-40805957 |
| chr1: 20512361-20512797 | chr12: 1770702-1771476 | chr17: 40912817-40913553 |
| chr1: 205537752-205538443 | chr12: 1905278-1906765 | chr17: 40932330-40933299 |
| chr1: 206223538-206224028 | chr12: 20521617-20523122 | chr17: 41723220-41723826 |
| chr1: 2064629-2064855 | chr12: 21680409-21680982 | chr17: 41791111-41791476 |
| chr1: 206730398-206730908 | chr12: 21810489-21810766 | chr17: 41984149-41985012 |
| chr1: 20810463-20813511 | chr12: 22486836-22488666 | chr17: 42015422-42015707 |
| chr1: 209848444-209849428 | chr12: 24714957-24716243 | chr17: 42015844-42016069 |
| chr1: 209979317-209979666 | chr12: 26348261-26349130 | chr17: 42030174-42030941 |
| chr1: 210465710-210466212 | chr12: 2800140-2801062 | chr17: 42061047-42061643 |
| chr1: 211306668-211307675 | chr12: 28127891-28128575 | chr17: 42082028-42084972 |
| chr1: 211688462-211689104 | chr12: 29935996-29937433 | chr17: 42091713-42091948 |
| chr1: 213123648-213125092 | chr12: 3862069-3862606 | chr17: 42092144-42092432 |
| chr1: 214161198-214161415 | chr12: 4273820-4274491 | chr17: 42287693-42288392 |
| chr1: 2144200-2144497 | chr12: 4378367-4382222 | chr17: 42392324-42393079 |
| chr1: 215256052-215256636 | chr12: 4383194-4384405 | chr17: 42402788-42403266 |
| chr1: 219347110-219347572 | chr12: 49318487-49319476 | chr17: 44026528-44026738 |
| chr1: 220960017-220960603 | chr12: 49363665-49364443 | chr17: 44848309-44849912 |
| chr1: 2222199-2222569 | chr12: 49390618-49392441 | chr17: 45400875-45401440 |
| chr1: 225117221-225117781 | chr12: 49487964-49488202 | chr17: 45928212-45928710 |
| chr1: 226270724-226271841 | chr12: 49688874-49691360 | chr17: 46089637-46089851 |
| chr1: 22668639-22668862 | chr12: 49735720-49736875 | chr17: 46114574-46115059 |
| chr1: 226736355-226737412 | chr12: 50297581-50297988 | chr17: 46507345-46507778 |
| chr1: 227729516-227730492 | chr12: 50349080-50349525 | chr17: 46655216-46655604 |
| chr1: 228565950-228567121 | chr12: 51785280-51785821 | chr17: 46687528-46688730 |
| chr1: 230561104-230562702 | chr12: 51818461-51819166 | chr17: 46710813-46711419 |
| chr1: 231175063-231176317 | chr12: 52444554-52445421 | chr17: 46719361-46720234 |
| chr1: 231176786-231177009 | chr12: 52545938-52546363 | chr17: 46723732-46724383 |
| chr1: 232941055-232941707 | chr12: 52701963-52702560 | chr17: 46755566-46756006 |
| chr1: 233749374-233750314 | chr12: 53267860-53268290 | chr17: 46827436-46827641 |
| chr1: 236687072-236687608 | chr12: 53273232-53273498 | chr17: 47209812-47210740 |
| chr1: 23750509-23751663 | chr12: 53297443-53297824 | chr17: 47572346-47575316 |
| chr1: 23884843-23885087 | chr12: 53441385-53441706 | chr17: 47647377-47647660 |
| chr1: 240254960-240257063 | chr12: 53448009-53448406 | chr17: 47967874-47968409 |
| chr1: 244012713-244013245 | chr12: 53613717-53615103 | chr17: 48619112-48619794 |
| chr1: 244213398-244213619 | chr12: 53718633-53719778 | chr17: 49021857-49022279 |
| chr1: 2460761-2462010 | chr12: 54332806-54333731 | chr17: 4981358-4981979 |
| chr1: 24648203-24648985 | chr12: 54343623-54343848 | chr17: 52977867-52978307 |
| chr1: 24739858-24740262 | chr12: 54346778-54347101 | chr17: 53315619-53316198 |
| chr1: 2477564-2478363 | chr12: 54387826-54388732 | chr17: 53342199-53343061 |
| chr1: 26490523-26491015 | chr12: 54389114-54389520 | chr17: 54674159-54674366 |
| chr1: 26686517-26687281 | chr12: 54393375-54394648 | chr17: 54910497-54912470 |
| chr1: 27338880-27339441 | chr12: 54399413-54399654 | chr17: 55939089-55939591 |
| chr1: 27854417-27854963 | chr12: 54399907-54400495 | chr17: 56832962-56833986 |
| chr1: 27894928-27895524 | chr12: 54408427-54408713 | chr17: 58228062-58228361 |
| chr1: 27960568-27961023 | chr12: 54784901-54785238 | chr17: 58498712-58499332 |
| chr1: 27986306-27986808 | chr12: 5541211-5542973 | chr17: 59473061-59483266 |
| chr1: 29101791-29102069 | chr12: 56522944-56523285 | chr17: 59539363-59539834 |
| chr1: 2929156-2929376 | chr12: 56881402-56882702 | chr17: 5973316-5974449 |
| chr1: 31158010-31158261 | chr12: 58013172-58013652 | chr17: 60704543-60705943 |
| chr1: 31380845-31381078 | chr12: 58021295-58022037 | chr17: 60729646-60730269 |
| chr1: 32169538-32169869 | chr12: 58258987-58259496 | chr17: 60885409-60885857 |
| chr1: 32180132-32180487 | chr12: 59989652-59990507 | chr17: 61523001-61524470 |
| chr1: 32226147-32226535 | chr12: 63025589-63026213 | chr17: 61615393-61616035 |
| chr1: 32237828-32238661 | chr12: 6419604-6420024 | chr17: 6460072-6460302 |
| chr1: 3239916-3240261 | chr12: 6472661-6473322 | chr17: 64961008-64962321 |
| chr1: 32410189-32410630 | chr12: 6492890-6493522 | chr17: 68164914-68165720 |
| chr1: 32892429-32892835 | chr12: 65672392-65673250 | chr17: 6945390-6947521 |
| chr1: 3310103-3311035 | chr12: 6664426-6665336 | chr17: 7108305-7108654 |
| chr1: 33219428-33220028 | chr12: 71003625-71004057 | chr17: 71639029-71641670 |
| chr1: 33358470-33359449 | chr12: 8123347-8123561 | chr17: 7164286-7166245 |
| chr1: 33438459-33438979 | chr12: 81330609-81331514 | chr17: 71897971-71898420 |
| chr1: 33938026-33938328 | chr12: 82152321-82152674 | chr17: 72270302-72270512 |
| chr1: 3447450-3447950 | chr12: 8849964-8851403 | chr17: 72352828-72353798 |
| chr1: 35331704-35332409 | chr12: 96883287-96883737 | chr17: 72442928-72443194 |
| chr1: 35350879-35351854 | chr12: 98850691-98851002 | chr17: 72449983-72450617 |
| chr1: 35394748-35396206 | chr12: 99288223-99289374 | chr17: 72667196-72667710 |
| chr1: 36042433-36043444 | chr13: 100631753-100635526 | chr17: 72732849-72733545 |
| chr1: 3662964-3664085 | chr13: 102068117-102069258 | chr17: 72848167-72848901 |
| chr1: 36771831-36773009 | chr13: 107186469-107189024 | chr17: 72889350-72890060 |
| chr1: 3688554-3689684 | chr13: 110958892-110960590 | chr17: 72931730-72932601 |
| chr1: 37498378-37500624 | chr13: 110960925-110961143 | chr17: 73030677-73031160 |
| chr1: 38229839-38230888 | chr13: 113548644-113549127 | chr17: 73083867-73084495 |
| chr1: 41847265-41849204 | chr13: 113597554-113598303 | chr17: 73520957-73522540 |
| chr1: 43832815-43833073 | chr13: 113622738-113623660 | chr17: 74496993-74497632 |
| chr1: 44401758-44402423 | chr13: 113807289-113807865 | chr17: 74706466-74707067 |
| chr1: 44871110-44874047 | chr13: 114017867-114018463 | chr17: 75524590-75525152 |
| chr1: 44883137-44884272 | chr13: 20175967-20176766 | chr17: 7608377-7608956 |
| chr1: 46767426-46769036 | chr13: 20702852-20703122 | chr17: 76127522-76128406 |
| chr1: 46859725-46860291 | chr13: 20806017-20806867 | chr17: 77093292-77093805 |
| chr1: 46913787-46914343 | chr13: 23489631-23490058 | chr17: 77783810-77789842 |
| chr1: 47489227-47489633 | chr13: 26760422-26760892 | chr17: 78977614-78978447 |
| chr1: 47690981-47691727 | chr13: 28491489-28492518 | chr17: 79366807-79374742 |
| chr1: 47915640-47915952 | chr13: 28501860-28502090 | chr17: 79448219-79448827 |
| chr1: 47998900-47999517 | chr13: 28542222-28543544 | chr17: 79485600-79486913 |
| chr1: 48058794-48059230 | chr13: 28554428-28555065 | chr17: 79614851-79615559 |
| chr1: 48190757-48190992 | chr13: 29105412-29105676 | chr17: 79917287-79920022 |
| chr1: 48449871-48450144 | chr13: 29292671-29293329 | chr17: 80329067-80330208 |
| chr1: 48462132-48462976 | chr13: 30995686-30995947 | chr17: 8054551-8055835 |
| chr1: 48937305-48937683 | chr13: 31019861-31020137 | chr17: 9142981-9143839 |
| chr1: 49242372-49242810 | chr13: 31377146-31377493 | chr18: 10131854-10132080 |
| chr1: 50513645-50514320 | chr13: 36705041-36705707 | chr18: 12271658-12271920 |
| chr1: 50798668-50799536 | chr13: 36871668-36872059 | chr18: 12407716-12408242 |
| chr1: 53386618-53387523 | chr13: 36919738-36921004 | chr18: 13136597-13137564 |
| chr1: 55446088-55446846 | chr13: 37005582-37006453 | chr18: 13824002-13824215 |
| chr1: 57110664-57111337 | chr13: 37247950-37248463 | chr18: 14132001-14132482 |
| chr1: 57887964-57890637 | chr13: 43597505-43597856 | chr18: 14430639-14431567 |
| chr1: 59280952-59281194 | chr13: 44359859-44361696 | chr18: 19928417-19929329 |
| chr1: 60280625-60281048 | chr13: 44453208-44454295 | chr18: 20139485-20140246 |
| chr1: 6086245-6086494 | chr13: 45885755-45886103 | chr18: 21199432-21199798 |
| chr1: 61508643-61509282 | chr13: 50070023-50070719 | chr18: 21269270-21270349 |
| chr1: 61519353-61519971 | chr13: 53174029-53174674 | chr18: 22006311-22007007 |
| chr1: 6208717-6209039 | chr13: 53424942-53425995 | chr18: 24443201-24443458 |
| chr1: 6241032-6241251 | chr13: 67804594-67805459 | chr18: 25755415-25758175 |
| chr1: 6265826-6266778 | chr13: 88323570-88324640 | chr18: 2846566-2848175 |
| chr1: 6301696-6302856 | chr13: 95201561-95202293 | chr18: 28681321-28682455 |
| chr1: 6484504-6485327 | chr13: 95354190-95355184 | chr18: 2905951-2907348 |
| chr1: 6507208-6509186 | chr13: 95619884-95620977 | chr18: 29077553-29078526 |
| chr1: 6545144-6545559 | chr13: 96204692-96205496 | chr18: 31802359-31803792 |
| chr1: 65468273-65468828 | chr13: 98794399-98796241 | chr18: 31804481-31804875 |
| chr1: 68696640-68697628 | chr14: 100204041-100204262 | chr18: 34823799-34824017 |
| chr1: 70032968-70034495 | chr14: 100625320-100626461 | chr18: 3498942-3499358 |
| chr1: 72748472-72749736 | chr14: 100680270-100680947 | chr18: 42258984-42260795 |
| chr1: 76080455-76080808 | chr14: 101012841-101013117 | chr18: 43355319-43355698 |
| chr1: 76540148-76540653 | chr14: 101121009-101121365 | chr18: 43913346-43914477 |
| chr1: 77333112-77334534 | chr14: 102247661-102248279 | chr18: 44526867-44527137 |
| chr1: 8002409-8002699 | chr14: 103010785-103011625 | chr18: 45662315-45663520 |
| chr1: 8013994-8014651 | chr14: 103389017-103390248 | chr18: 47087076-47087446 |
| chr1: 805199-805628 | chr14: 103394398-103397070 | chr18: 47087880-47088528 |
| chr1: 82265999-82269048 | chr14: 103589354-103590246 | chr18: 48085839-48087589 |
| chr1: 8277196-8277822 | chr14: 103739579-103740892 | chr18: 5237724-5238674 |
| chr1: 84326268-84326838 | chr14: 105293561-105293879 | chr18: 52495605-52496007 |
| chr1: 85462586-85463435 | chr14: 105309691-105310500 | chr18: 52626518-52626849 |
| chr1: 87617154-87617417 | chr14: 105330515-105332186 | chr18: 53447075-53447842 |
| chr1: 87617693-87617986 | chr14: 105512056-105512307 | chr18: 5542848-5543054 |
| chr1: 90308840-90309606 | chr14: 105662778-105663403 | chr18: 55862654-55862873 |
| chr1: 9258566-9258956 | chr14: 105952604-105954296 | chr18: 56932714-56932994 |
| chr1: 94702691-94703344 | chr14: 21100839-21101043 | chr18: 56934834-56936827 |
| chr1: 95285603-95286319 | chr14: 21537083-21537355 | chr18: 5890293-5891682 |
| chr1: 9749296-9750228 | chr14: 21539712-21540045 | chr18: 59992070-59993556 |
| chr1: 999679-999911 | chr14: 23355739-23356402 | chr18: 63417776-63418640 |
| chr10: 100227439-100227832 | chr14: 24457865-24458100 | chr18: 65183238-65184014 |
| chr10: 101293016-101293238 | chr14: 24550318-24551148 | chr18: 70208975-70211790 |
| chr10: 102416497-102416716 | chr14: 24835811-24836220 | chr18: 72123607-72124717 |
| chr10: 102430698-102431119 | chr14: 24837873-24838324 | chr18: 74514041-74514292 |
| chr10: 102440601-102441011 | chr14: 24898780-24899288 | chr18: 74843361-74845426 |
| chr10: 102469807-102470341 | chr14: 25518425-25519612 | chr18: 7566558-7568830 |
| chr10: 102501701-102502232 | chr14: 27066314-27066578 | chr18: 77724353-77724836 |
| chr10: 102586126-102588109 | chr14: 32670418-32671092 | chr18: 811650-812709 |
| chr10: 102590123-102590402 | chr14: 34419509-34420438 | chr18: 8367274-8367599 |
| chr10: 102778605-102778922 | chr14: 37641233-37641879 | chr18: 9333897-9334244 |
| chr10: 102792043-102792266 | chr14: 38052646-38054166 | chr19: 10077032-10077444 |
| chr10: 102807775-102808271 | chr14: 38057139-38057798 | chr19: 10120751-10121231 |
| chr10: 102809888-102810282 | chr14: 38060842-38062119 | chr19: 10526983-10527755 |
| chr10: 102882978-102883551 | chr14: 38063664-38065665 | chr19: 10529628-10532004 |
| chr10: 103326283-103326712 | chr14: 38067447-38069207 | chr19: 1068439-1068764 |
| chr10: 103538816-103539988 | chr14: 45431629-45432115 | chr19: 10697787-10698099 |
| chr10: 103985853-103986246 | chr14: 52780688-52781969 | chr19: 1074727-1075071 |
| chr10: 104000255-104001741 | chr14: 54413305-54413764 | chr19: 11353961-11354362 |
| chr10: 105211951-105212306 | chr14: 55595698-55596692 | chr19: 11491560-11492740 |
| chr10: 106028543-106029047 | chr14: 58862542-58863209 | chr19: 11529214-11529588 |
| chr10: 110225928-110226465 | chr14: 60097209-60097553 | chr19: 11529723-11529966 |
| chr10: 11059443-11060524 | chr14: 60794577-60794867 | chr19: 11531279-11531590 |
| chr10: 11207179-11207980 | chr14: 61114103-61116552 | chr19: 11533199-11533619 |
| chr10: 113943284-113943657 | chr14: 61746805-61748141 | chr19: 11689418-11689768 |
| chr10: 115860680-115861093 | chr14: 61787881-61789467 | chr19: 11909403-11909841 |
| chr10: 116391467-116391827 | chr14: 62583680-62584279 | chr19: 11959578-11960064 |
| chr10: 116527525-116528474 | chr14: 67878535-67879167 | chr19: 11998805-11999131 |
| chr10: 116852262-116854094 | chr14: 70041214-70041662 | chr19: 12076030-12076366 |
| chr10: 118976050-118976706 | chr14: 73704055-73705106 | chr19: 12098572-12098950 |
| chr10: 119293918-119297675 | chr14: 73706300-73706677 | chr19: 12146031-12146649 |
| chr10: 122708511-122708899 | chr14: 73957751-73958889 | chr19: 12163327-12163560 |
| chr10: 122739053-122739391 | chr14: 74036128-74036662 | chr19: 12175461-12176057 |
| chr10: 124638744-124639793 | chr14: 74058401-74059116 | chr19: 12266999-12267686 |
| chr10: 125851152-125853233 | chr14: 74100344-74101230 | chr19: 12305553-12306304 |
| chr10: 126135810-126138896 | chr14: 75389713-75390133 | chr19: 12444033-12444548 |
| chr10: 133879006-133879255 | chr14: 75894309-75895469 | chr19: 12476050-12476372 |
| chr10: 134973511-134974311 | chr14: 76819146-76819379 | chr19: 12943347-12943661 |
| chr10: 135075002-135075452 | chr14: 88789601-88790493 | chr19: 12978360-12978785 |
| chr10: 135089970-135090491 | chr14: 89493136-89494382 | chr19: 13207376-13207621 |
| chr10: 13933483-13934184 | chr14: 90167783-90168352 | chr19: 13208344-13210525 |
| chr10: 17270431-17272617 | chr14: 91883362-91883566 | chr19: 1324970-1325349 |
| chr10: 17495408-17496721 | chr14: 92302202-92303078 | chr19: 13266390-13267030 |
| chr10: 18429126-18430234 | chr14: 92413582-92414324 | chr19: 1401093-1401784 |
| chr10: 20104452-20106105 | chr14: 96505312-96506402 | chr19: 14168143-14168512 |
| chr10: 20106644-20106946 | chr14: 96890431-96890633 | chr19: 14542769-14543102 |
| chr10: 21462129-21463808 | chr14: 97685060-97685946 | chr19: 1455227-1455544 |
| chr10: 22518027-22518334 | chr14: 99711840-99713431 | chr19: 1456078-1456347 |
| chr10: 25241111-25241900 | chr14: 99739802-99740920 | chr19: 1456886-1457312 |
| chr10: 25463757-25465639 | chr15: 100880958-100882438 | chr19: 1503512-1503791 |
| chr10: 26222922-26224022 | chr15: 100890243-100890494 | chr19: 15090017-15090873 |
| chr10: 26500608-26501126 | chr15: 101389733-101390260 | chr19: 15288315-15288911 |
| chr10: 26680635-26680999 | chr15: 101548676-101548898 | chr19: 15334123-15334822 |
| chr10: 28030183-28035211 | chr15: 23207950-23208796 | chr19: 15619224-15619613 |
| chr10: 29698363-29699044 | chr15: 24123232-24123432 | chr19: 15662114-15662358 |
| chr10: 31422710-31423316 | chr15: 28982670-28983497 | chr19: 15695299-15695535 |
| chr10: 35103125-35105243 | chr15: 30113706-30113940 | chr19: 16186790-16188275 |
| chr10: 43571940-43573434 | chr15: 31733082-31733489 | chr19: 1675922-1676137 |
| chr10: 44185040-44186132 | chr15: 32828333-32829181 | chr19: 17007102-17008857 |
| chr10: 45470023-45470291 | chr15: 34728788-34729495 | chr19: 1725416-1726154 |
| chr10: 45914375-45914883 | chr15: 37179517-37179782 | chr19: 17346166-17346886 |
| chr10: 49812853-49813352 | chr15: 37393667-37394248 | chr19: 17392384-17393775 |
| chr10: 51572199-51572620 | chr15: 40728263-40728466 | chr19: 17403040-17403570 |
| chr10: 52177725-52178182 | chr15: 41851724-41851970 | chr19: 17487896-17488125 |
| chr10: 57390344-57391215 | chr15: 41913334-41914360 | chr19: 17501956-17502250 |
| chr10: 60272468-60273456 | chr15: 41952536-41953222 | chr19: 17580198-17580598 |
| chr10: 60935828-60937049 | chr15: 45427297-45428206 | chr19: 1762373-1762848 |
| chr10: 62492786-62493376 | chr15: 45722562-45722939 | chr19: 1766145-1766364 |
| chr10: 62703308-62704290 | chr15: 48009419-48011044 | chr19: 17716645-17717531 |
| chr10: 64133790-64134420 | chr15: 48470008-48470628 | chr19: 1776028-1776730 |
| chr10: 71077947-71079377 | chr15: 51633725-51634318 | chr19: 17791068-17791301 |
| chr10: 71812319-71813693 | chr15: 52587354-52588172 | chr19: 17905580-17906317 |
| chr10: 71892093-71892802 | chr15: 60690709-60690930 | chr19: 18059850-18060148 |
| chr10: 72043447-72043886 | chr15: 61519622-61520031 | chr19: 18209912-18210268 |
| chr10: 72218131-72218484 | chr15: 61520424-61521716 | chr19: 18319146-18319363 |
| chr10: 72647739-72648317 | chr15: 62456016-62457648 | chr19: 18499098-18499697 |
| chr10: 72977124-72977774 | chr15: 64443588-64444821 | chr19: 18539782-18540341 |
| chr10: 734708-735606 | chr15: 65066917-65068352 | chr19: 1862257-1862600 |
| chr10: 74020774-74021023 | chr15: 65127965-65128185 | chr19: 18722359-18724001 |
| chr10: 74079337-74079861 | chr15: 65669311-65670617 | chr19: 18811562-18811791 |
| chr10: 75670697-75671379 | chr15: 65714743-65715571 | chr19: 19280999-19281560 |
| chr10: 77191062-77191571 | chr15: 66274584-66274838 | chr19: 19335772-19336151 |
| chr10: 79396096-79398495 | chr15: 66544433-66546134 | chr19: 19729128-19729814 |
| chr10: 8091375-8098329 | chr15: 68126023-68126569 | chr19: 19738573-19739821 |
| chr10: 81892223-81892640 | chr15: 68723368-68724691 | chr19: 19843483-19843943 |
| chr10: 85954200-85955303 | chr15: 69366322-69366796 | chr19: 19971578-19971863 |
| chr10: 86300400-86300953 | chr15: 71145996-71146820 | chr19: 20011911-20012238 |
| chr10: 88730555-88731632 | chr15: 72476677-72476969 | chr19: 20149856-20150175 |
| chr10: 90342529-90343221 | chr15: 72564443-72565165 | chr19: 20162773-20163107 |
| chr10: 93647053-93647393 | chr15: 72611947-72612802 | chr19: 2041824-2042593 |
| chr10: 94351357-94351615 | chr15: 74044562-74045885 | chr19: 21949904-21950217 |
| chr10: 94459255-94459591 | chr15: 74425092-74428821 | chr19: 22018595-22018827 |
| chr10: 94831513-94832312 | chr15: 74537810-74538114 | chr19: 22034395-22034943 |
| chr10: 95326230-95327108 | chr15: 75470912-75471212 | chr19: 22193331-22193583 |
| chr10: 95360390-95361387 | chr15: 75628251-75628677 | chr19: 2273694-2274047 |
| chr10: 95753348-95754201 | chr15: 78633323-78634089 | chr19: 2302467-2302999 |
| chr10: 97802872-97804262 | chr15: 79102716-79104628 | chr19: 23253797-23254382 |
| chr10: 98479726-98480487 | chr15: 83348840-83349865 | chr19: 23386650-23387069 |
| chr10: 99473085-99473291 | chr15: 83620951-83621727 | chr19: 23456575-23456868 |
| chr11: 100998031-100999774 | chr15: 83775862-83776922 | chr19: 2478866-2479244 |
| chr11: 101918074-101918493 | chr15: 83875649-83877079 | chr19: 2613933-2614133 |
| chr11: 102188180-102188440 | chr15: 84047823-84048105 | chr19: 267086-267660 |
| chr11: 106888324-106890203 | chr15: 84115763-84116921 | chr19: 28284529-28285129 |
| chr11: 109963241-109964677 | chr15: 84322323-84323771 | chr19: 295115-295354 |
| chr11: 112832525-112834490 | chr15: 84976071-84977044 | chr19: 30215233-30215594 |
| chr11: 113185333-113185663 | chr15: 89438177-89438850 | chr19: 30363203-30363527 |
| chr11: 113258444-113258821 | chr15: 89942593-89943853 | chr19: 31839636-31843049 |
| chr11: 113345069-113346328 | chr15: 89959743-89959990 | chr19: 31843881-31844183 |
| chr11: 113929634-113932190 | chr15: 90039465-90039984 | chr19: 31847947-31848209 |
| chr11: 113953621-113953839 | chr15: 91427541-91427905 | chr19: 3275609-3275970 |
| chr11: 116371183-116371606 | chr15: 93198375-93199181 | chr19: 33685147-33686085 |
| chr11: 116706482-116706910 | chr15: 93615623-93617185 | chr19: 34112280-34114353 |
| chr11: 119227097-119227758 | chr15: 96883942-96884533 | chr19: 3434930-3435417 |
| chr11: 119455154-119456102 | chr15: 96888959-96889417 | chr19: 34396472-34398079 |
| chr11: 119612092-119612476 | chr15: 99091356-99091812 | chr19: 34850072-34850587 |
| chr11: 120039602-120040210 | chr15: 99557979-99559170 | chr19: 35531781-35531991 |
| chr11: 120110498-120110719 | chr16: 10479687-10480254 | chr19: 36164078-36164286 |
| chr11: 120856726-120857174 | chr16: 1098523-1098875 | chr19: 36351441-36352167 |
| chr11: 121322539-121323302 | chr16: 11327009-11327234 | chr19: 3671903-3672121 |
| chr11: 122854931-122855582 | chr16: 1202415-1204624 | chr19: 3687787-3688262 |
| chr11: 123301050-123302149 | chr16: 12897366-12897889 | chr19: 36909282-36909854 |
| chr11: 124709223-124709957 | chr16: 1392545-1392753 | chr19: 37063893-37064749 |
| chr11: 124713006-124713395 | chr16: 1429051-1430104 | chr19: 37095681-37096589 |
| chr11: 124932731-124933657 | chr16: 1458144-1458435 | chr19: 37157633-37158119 |
| chr11: 126225356-126226073 | chr16: 19421817-19422498 | chr19: 3721465-3721788 |
| chr11: 126286452-126286870 | chr16: 203754-204866 | chr19: 37263382-37264083 |
| chr11: 12695415-12696981 | chr16: 2054012-2054288 | chr19: 37328897-37329518 |
| chr11: 128391713-128392611 | chr16: 2069719-2070865 | chr19: 37340919-37341262 |
| chr11: 129243999-129244567 | chr16: 2077043-2077470 | chr19: 37341732-37342000 |
| chr11: 129245109-129246395 | chr16: 21294981-21295414 | chr19: 37568953-37569549 |
| chr11: 129685738-129686211 | chr16: 21831483-21831944 | chr19: 37803517-37803822 |
| chr11: 130029659-130030262 | chr16: 2245867-2247100 | chr19: 37957727-37958390 |
| chr11: 130297402-130298517 | chr16: 25078048-25078532 | chr19: 37959853-37960615 |
| chr11: 133825338-133827457 | chr16: 2835064-2835958 | chr19: 38145827-38147219 |
| chr11: 134201785-134202407 | chr16: 28634441-28635031 | chr19: 38182794-38183327 |
| chr11: 13689589-13690724 | chr16: 28752083-28752332 | chr19: 38210108-38210692 |
| chr11: 15094958-15095872 | chr16: 29624388-29625391 | chr19: 3821975-3822278 |
| chr11: 1592499-1592810 | chr16: 29887789-29888316 | chr19: 38754847-38755699 |
| chr11: 16632509-16632725 | chr16: 30428871-30429799 | chr19: 38852312-38853485 |
| chr11: 17373020-17373665 | chr16: 3078142-3078553 | chr19: 39359917-39361160 |
| chr11: 17565767-17566052 | chr16: 3079537-3079954 | chr19: 39440405-39441011 |
| chr11: 18067717-18067928 | chr16: 30913356-30913942 | chr19: 39574701-39575137 |
| chr11: 18415923-18416680 | chr16: 31008833-31009036 | chr19: 39687598-39687964 |
| chr11: 18727321-18727727 | chr16: 31117102-31117435 | chr19: 39810993-39811293 |
| chr11: 19798538-19798949 | chr16: 31213567-31214287 | chr19: 39894559-39894768 |
| chr11: 20385162-20385673 | chr16: 31342453-31343140 | chr19: 39993358-39993765 |
| chr11: 2165136-2165672 | chr16: 31483277-31483646 | chr19: 39997591-39998291 |
| chr11: 22362863-22363377 | chr16: 31580071-31580317 | chr19: 40323908-40325005 |
| chr11: 22850766-22851367 | chr16: 31580560-31581023 | chr19: 40732076-40732665 |
| chr11: 2290105-2292932 | chr16: 3199653-3199937 | chr19: 41025315-41026106 |
| chr11: 2465172-2465648 | chr16: 3225355-3225594 | chr19: 41055125-41055331 |
| chr11: 2466248-2466818 | chr16: 32822902-32823585 | chr19: 41060059-41060565 |
| chr11: 27740645-27741236 | chr16: 3355021-3356012 | chr19: 41106059-41106415 |
| chr11: 30040073-30040294 | chr16: 4166656-4167012 | chr19: 41109614-41110075 |
| chr11: 30606005-30608128 | chr16: 4357553-4357955 | chr19: 41111084-41111767 |
| chr11: 3181575-3182120 | chr16: 4363936-4364151 | chr19: 41115446-41115767 |
| chr11: 32354761-32355484 | chr16: 4377509-4378162 | chr19: 41119032-41120394 |
| chr11: 32448262-32449744 | chr16: 4421643-4422240 | chr19: 41633777-41634047 |
| chr11: 35547140-35547396 | chr16: 4587837-4588890 | chr19: 41698744-41699325 |
| chr11: 416420-417650 | chr16: 46823933-46824263 | chr19: 42419339-42419564 |
| chr11: 43596991-43597336 | chr16: 47176788-47178446 | chr19: 42501262-42501486 |
| chr11: 43902255-43902528 | chr16: 48399731-48400040 | chr19: 42720827-42722132 |
| chr11: 45307442-45307975 | chr16: 50581476-50582735 | chr19: 42901017-42901375 |
| chr11: 45686161-45687495 | chr16: 52579637-52580845 | chr19: 42905724-42906350 |
| chr11: 45921388-45922167 | chr16: 52581294-52581610 | chr19: 42927286-42928598 |
| chr11: 46316876-46317485 | chr16: 54316904-54317149 | chr19: 43979360-43979684 |
| chr11: 46389075-46389378 | chr16: 54962423-54967805 | chr19: 44324437-44325009 |
| chr11: 47736740-47737106 | chr16: 55357522-55359040 | chr19: 44405818-44406053 |
| chr11: 49229718-49230040 | chr16: 56224732-56224980 | chr19: 44763979-44764312 |
| chr11: 504190-504659 | chr16: 56225256-56226351 | chr19: 44952417-44952809 |
| chr11: 57117316-57117575 | chr16: 56677121-56677598 | chr19: 45257704-45257918 |
| chr11: 57243682-57244463 | chr16: 56701864-56702208 | chr19: 45281134-45281355 |
| chr11: 57249947-57250848 | chr16: 56703388-56703648 | chr19: 4535070-4535339 |
| chr11: 58940831-58941052 | chr16: 57835956-57836989 | chr19: 4558364-4558579 |
| chr11: 61061752-61063063 | chr16: 58058713-58058928 | chr19: 45655294-45657246 |
| chr11: 61159837-61160285 | chr16: 58535041-58535596 | chr19: 45843871-45844418 |
| chr11: 61283865-61284147 | chr16: 66638255-66639561 | chr19: 45888769-45889465 |
| chr11: 61322733-61323285 | chr16: 66982435-66982945 | chr19: 45947711-45948047 |
| chr11: 61335113-61335340 | chr16: 67034471-67035340 | chr19: 46032305-46032855 |
| chr11: 61355039-61355363 | chr16: 67188614-67189652 | chr19: 4607080-4607350 |
| chr11: 61519742-61519972 | chr16: 67193006-67193219 | chr19: 46144873-46145631 |
| chr11: 61722745-61723483 | chr16: 67196793-67199988 | chr19: 46282545-46283127 |
| chr11: 62476771-62477481 | chr16: 67204209-67204637 | chr19: 46379884-46380207 |
| chr11: 62690975-62691488 | chr16: 67211934-67212509 | chr19: 46456210-46456503 |
| chr11: 62693374-62694772 | chr16: 67312897-67313731 | chr19: 46518284-46520080 |
| chr11: 63685490-63685968 | chr16: 67564059-67564332 | chr19: 46974558-46975073 |
| chr11: 63775347-63775613 | chr16: 67700010-67701354 | chr19: 46996328-46998437 |
| chr11: 63803376-63804365 | chr16: 68002838-68003039 | chr19: 47137781-47138070 |
| chr11: 64038965-64039306 | chr16: 68273546-68274084 | chr19: 47614409-47614661 |
| chr11: 64409878-64411253 | chr16: 68678687-68680149 | chr19: 47910108-47910563 |
| chr11: 64993281-64993484 | chr16: 69760294-69760506 | chr19: 47922252-47922777 |
| chr19: 48216487-48216837 | chr22: 23522551-23524465 | chr6: 10410110-10410690 |
| chr19: 48833395-48833720 | chr22: 23908323-23909178 | chr6: 10414217-10414498 |
| chr19: 48837142-48837549 | chr22: 24110030-24110995 | chr6: 10415019-10415318 |
| chr19: 48901805-48902123 | chr22: 24551814-24552696 | chr6: 10417385-10417842 |
| chr19: 49061546-49061769 | chr22: 26565200-26565986 | chr6: 10419400-10420323 |
| chr19: 49199965-49200184 | chr22: 27152698-27153133 | chr6: 10421415-10421727 |
| chr19: 49242019-49242962 | chr22: 29426511-29426843 | chr6: 10422322-10422648 |
| chr19: 49255779-49256495 | chr22: 29467292-29467817 | chr6: 10426201-10426638 |
| chr19: 49340489-49340774 | chr22: 31218227-31218893 | chr6: 105627407-105627830 |
| chr19: 49522774-49523093 | chr22: 32026298-32026874 | chr6: 106958233-106958718 |
| chr19: 49575130-49576076 | chr22: 33453893-33454505 | chr6: 106959765-106960985 |
| chr19: 49646093-49646308 | chr22: 37655606-37655839 | chr6: 108436110-108436522 |
| chr19: 49655103-49655395 | chr22: 37730552-37731415 | chr6: 108455061-108455273 |
| chr19: 49935752-49936275 | chr22: 38073038-38073412 | chr6: 112575092-112575483 |
| chr19: 49939611-49940045 | chr22: 38453188-38453503 | chr6: 117584585-117584816 |
| chr19: 50016532-50016851 | chr22: 38808785-38809017 | chr6: 118241348-118241569 |
| chr19: 50030982-50031300 | chr22: 39745984-39746634 | chr6: 124124178-124125309 |
| chr19: 50037079-50037670 | chr22: 39853324-39854065 | chr6: 125283125-125284389 |
| chr19: 50096610-50096912 | chr22: 40057942-40058844 | chr6: 125420744-125421141 |
| chr19: 50183923-50184172 | chr22: 40390485-40391490 | chr6: 127836731-127837706 |
| chr19: 50312012-50312821 | chr22: 42062791-42063002 | chr6: 1311069-1314220 |
| chr19: 50651174-50651535 | chr22: 42470036-42470669 | chr6: 132271357-132271658 |
| chr19: 50706483-50707196 | chr22: 43505952-43506167 | chr6: 134159127-134159349 |
| chr19: 50708602-50708855 | chr22: 45015934-45016488 | chr6: 134213960-134214351 |
| chr19: 50831455-50832070 | chr22: 45403037-45406372 | chr6: 13873766-13874262 |
| chr19: 50833814-50834128 | chr22: 46262066-46263747 | chr6: 13925100-13925510 |
| chr19: 50836391-50837447 | chr22: 46366727-46368726 | chr6: 146136326-146136564 |
| chr19: 51069360-51069600 | chr22: 50328694-50329178 | chr6: 150246753-150247257 |
| chr19: 51161924-51162514 | chr22: 50453057-50454137 | chr6: 150284683-150286515 |
| chr19: 51198809-51199347 | chr22: 50628950-50629249 | chr6: 150463772-150465002 |
| chr19: 51227662-51228883 | chr22: 50982571-50982994 | chr6: 151561284-151562550 |
| chr19: 51321268-51321781 | chr3: 101497831-101498648 | chr6: 151646669-151646958 |
| chr19: 51416003-51416249 | chr3: 101568108-101569046 | chr6: 152128823-152129771 |
| chr19: 51522005-51522803 | chr3: 10206450-10207024 | chr6: 152623280-152623480 |
| chr19: 51568155-51568412 | chr3: 112930438-112931506 | chr6: 152957811-152958472 |
| chr19: 51842129-51842353 | chr3: 113251700-113252290 | chr6: 155316300-155317197 |
| chr19: 52206245-52206726 | chr3: 117716101-117716564 | chr6: 158957221-158958677 |
| chr19: 52207133-52207731 | chr3: 119041383-119042255 | chr6: 159290674-159291195 |
| chr19: 52222424-52223208 | chr3: 119421856-119422334 | chr6: 1604607-1615866 |
| chr19: 52452317-52452543 | chr3: 120003884-120004426 | chr6: 166267510-166268461 |
| chr19: 52531324-52531724 | chr3: 120626881-120627579 | chr6: 166401527-166402659 |
| chr19: 52800400-52800871 | chr3: 122640714-122641355 | chr6: 17102310-17102539 |
| chr19: 52839445-52839937 | chr3: 12328995-12329227 | chr6: 19837505-19839314 |
| chr19: 52900956-52901158 | chr3: 124303530-124304092 | chr6: 21594614-21596437 |
| chr19: 5293011-5294211 | chr3: 124931162-124931747 | chr6: 21664508-21665178 |
| chr19: 53030825-53031359 | chr3: 126242784-126243358 | chr6: 21665716-21666031 |
| chr19: 53073309-53074039 | chr3: 127391012-127392434 | chr6: 21666656-21666862 |
| chr19: 53104385-53105149 | chr3: 128145258-128145656 | chr6: 24360030-24360411 |
| chr19: 53141176-53141813 | chr3: 128205496-128212274 | chr6: 25652381-25652709 |
| chr19: 53193140-53193945 | chr3: 128336407-128337113 | chr6: 26020672-26021125 |
| chr19: 5339641-5341061 | chr3: 128564772-128565007 | chr6: 26021958-26022193 |
| chr19: 53400461-53401205 | chr3: 129323916-129325332 | chr6: 26044204-26044469 |
| chr19: 53426079-53426389 | chr3: 129345668-129346521 | chr6: 26045645-26046033 |
| chr19: 53496733-53497028 | chr3: 129721369-129722131 | chr6: 26172034-26172398 |
| chr19: 53606060-53606784 | chr3: 13008649-13009210 | chr6: 26184037-26184336 |
| chr19: 53635626-53636230 | chr3: 131753881-131754139 | chr6: 26197071-26197537 |
| chr19: 53661431-53661848 | chr3: 133464950-133465420 | chr6: 26199198-26199466 |
| chr19: 53662190-53662500 | chr3: 134082861-134083201 | chr6: 26204765-26205213 |
| chr19: 53696030-53696650 | chr3: 134125488-134125871 | chr6: 26217212-26217521 |
| chr19: 54023869-54024560 | chr3: 136751342-136751683 | chr6: 26240698-26240951 |
| chr19: 54024646-54024923 | chr3: 138153270-138154621 | chr6: 26250437-26250827 |
| chr19: 54369388-54369809 | chr3: 140769887-140771080 | chr6: 26251899-26252233 |
| chr19: 54382665-54382951 | chr3: 145878431-145879287 | chr6: 26271346-26271588 |
| chr19: 54409967-54410200 | chr3: 145968335-145969008 | chr6: 26272471-26272696 |
| chr19: 54599161-54599456 | chr3: 150802997-150805168 | chr6: 26273291-26273557 |
| chr19: 54665922-54666377 | chr3: 152552603-152553712 | chr6: 26987599-26988074 |
| chr19: 54982389-54982897 | chr3: 156533840-156535131 | chr6: 27100175-27101124 |
| chr19: 5536532-5536789 | chr3: 157155268-157155826 | chr6: 27107139-27107394 |
| chr19: 55591434-55591644 | chr3: 158288801-158289271 | chr6: 27598688-27599146 |
| chr19: 55591906-55593980 | chr3: 159756633-159756997 | chr6: 27777856-27778251 |
| chr19: 55667533-55668465 | chr3: 160822495-160823260 | chr6: 27782248-27782485 |
| chr19: 55672024-55673212 | chr3: 168864031-168864449 | chr6: 27791781-27792217 |
| chr19: 5567519-5567939 | chr3: 169482339-169483052 | chr6: 27798991-27799427 |
| chr19: 55865017-55865434 | chr3: 169487146-169487522 | chr6: 27835191-27835461 |
| chr19: 55865825-55866324 | chr3: 173113271-173113681 | chr6: 28367125-28367651 |
| chr19: 55953372-55954641 | chr3: 179168736-179169593 | chr6: 2841811-2842273 |
| chr19: 56159258-56159937 | chr3: 179754521-179755245 | chr6: 2875602-2875811 |
| chr19: 56728589-56729375 | chr3: 181413015-181414022 | chr6: 2971087-2971962 |
| chr19: 56879418-56879995 | chr3: 181430142-181431076 | chr6: 3231596-3232289 |
| chr19: 56904637-56905355 | chr3: 181437185-181437478 | chr6: 32632159-32633027 |
| chr19: 56915357-56915856 | chr3: 182896921-182897381 | chr6: 33539023-33539339 |
| chr19: 57049676-57050646 | chr3: 182971430-182972635 | chr6: 36098040-36098621 |
| chr19: 57078366-57079301 | chr3: 183542497-183543804 | chr6: 39196818-39197517 |
| chr19: 57106576-57106896 | chr3: 184319377-184319822 | chr6: 40554596-40555928 |
| chr19: 57149424-57149631 | chr3: 184320006-184320218 | chr6: 40995803-40996241 |
| chr19: 57276615-57276942 | chr3: 184321784-184322277 | chr6: 41339237-41340027 |
| chr19: 57702697-57703425 | chr3: 18485113-18487056 | chr6: 41410590-41411140 |
| chr19: 57831600-57832250 | chr3: 184971642-184972002 | chr6: 41438063-41438594 |
| chr19: 57862442-57863236 | chr3: 185000558-185000896 | chr6: 42109905-42110361 |
| chr19: 57874764-57875110 | chr3: 186490603-186490813 | chr6: 4282005-4282310 |
| chr19: 57999026-57999613 | chr3: 186648175-186649103 | chr6: 42928219-42928810 |
| chr19: 58011125-58011743 | chr3: 187455331-187455843 | chr6: 43021126-43021694 |
| chr19: 5802235-5802569 | chr3: 187456251-187456580 | chr6: 43044045-43045057 |
| chr19: 58038573-58039208 | chr3: 187457732-187457948 | chr6: 43142014-43142217 |
| chr19: 58070554-58071273 | chr3: 188665276-188665552 | chr6: 43237261-43237643 |
| chr19: 58111230-58111770 | chr3: 193720954-193721291 | chr6: 43970074-43970922 |
| chr19: 58125531-58125902 | chr3: 193776092-193776308 | chr6: 44119527-44119781 |
| chr19: 58175624-58176104 | chr3: 193858771-193859695 | chr6: 44187187-44187400 |
| chr19: 58220190-58220517 | chr3: 194117602-194118988 | chr6: 45387347-45388164 |
| chr19: 58280927-58281486 | chr3: 194207386-194208785 | chr6: 4775132-4777550 |
| chr19: 58446337-58446800 | chr3: 196255496-196256013 | chr6: 49518316-49519186 |
| chr19: 58458687-58459219 | chr3: 197281606-197283128 | chr6: 52441270-52441793 |
| chr19: 58609339-58609988 | chr3: 197391890-197392481 | chr6: 53212618-53214043 |
| chr19: 58661738-58662287 | chr3: 23244051-23245071 | chr6: 54711154-54712033 |
| chr19: 58666024-58666833 | chr3: 24535845-24537436 | chr6: 56407465-56407926 |
| chr19: 58739944-58740554 | chr3: 24870675-24871369 | chr6: 56818874-56820308 |
| chr19: 58858454-58859223 | chr3: 27410613-27411066 | chr6: 69345217-69345483 |
| chr19: 58951215-58952250 | chr3: 27756213-27756422 | chr6: 70576975-70577572 |
| chr19: 59025309-59025705 | chr3: 27756661-27756941 | chr6: 710835-711297 |
| chr19: 6199293-6199551 | chr3: 32021970-32023364 | chr6: 7141037-7141576 |
| chr19: 6463992-6464780 | chr3: 32443153-32443455 | chr6: 72129509-72130756 |
| chr19: 6530827-6531552 | chr3: 38035702-38036000 | chr6: 73972820-73973027 |
| chr19: 6740670-6741203 | chr3: 38040290-38040535 | chr6: 74019429-74020053 |
| chr19: 675672-675881 | chr3: 38080629-38081187 | chr6: 74024403-74025020 |
| chr19: 6767946-6768160 | chr3: 38179858-38180689 | chr6: 74161087-74162090 |
| chr19: 681346-681576 | chr3: 40428652-40429015 | chr6: 74233269-74233546 |
| chr19: 7953281-7953708 | chr3: 40657160-40657395 | chr6: 75794881-75795088 |
| chr19: 8273531-8273920 | chr3: 44690127-44690587 | chr6: 75914706-75916387 |
| chr19: 8274242-8275045 | chr3: 44726929-44727237 | chr6: 83073417-83075319 |
| chr19: 9473590-9474001 | chr3: 44754100-44754399 | chr6: 87647254-87647707 |
| chr19: 9896564-9896994 | chr3: 44770937-44771137 | chr6: 94126273-94129677 |
| chr2: 100937780-100939059 | chr3: 45187027-45187946 | chr6: 96463870-96464136 |
| chr2: 101033607-101034296 | chr3: 46734732-46735717 | chr6: 99279318-99283842 |
| chr2: 10260280-10260931 | chr3: 46742765-46743086 | chr7: 100091181-100091598 |
| chr2: 102758807-102759577 | chr3: 46887629-46887919 | chr7: 100167137-100168144 |
| chr2: 105488369-105489991 | chr3: 46940055-46940394 | chr7: 100201615-100203652 |
| chr2: 105760110-105761018 | chr3: 50275394-50275645 | chr7: 100224271-100224558 |
| chr2: 106886119-106886738 | chr3: 50377804-50378540 | chr7: 100230650-100231320 |
| chr2: 107502354-107504216 | chr3: 50402104-50402942 | chr7: 100434983-100435193 |
| chr2: 10861207-10862382 | chr3: 50604906-50605359 | chr7: 100845051-100845726 |
| chr2: 110370907-110373301 | chr3: 51989764-51990639 | chr7: 102157824-102158510 |
| chr2: 110518072-110518913 | chr3: 52279602-52280140 | chr7: 102329951-102330654 |
| chr2: 112656187-112656918 | chr3: 53190510-53190764 | chr7: 104624338-104624848 |
| chr2: 119981080-119981818 | chr3: 54155240-54157025 | chr7: 106684814-106685765 |
| chr2: 121101047-121101432 | chr3: 55515153-55515613 | chr7: 108095402-108097206 |
| chr2: 121101801-121104534 | chr3: 55517659-55517939 | chr7: 114562171-114563193 |
| chr2: 121104713-121104935 | chr3: 55519090-55519623 | chr7: 115850371-115851028 |
| chr2: 121199724-121199993 | chr3: 55520227-55522344 | chr7: 116139775-116140352 |
| chr2: 121200504-121200788 | chr3: 55522561-55522836 | chr7: 121513047-121513911 |
| chr2: 121499229-121499578 | chr3: 56501869-56502345 | chr7: 12610166-12610834 |
| chr2: 121624828-121625209 | chr3: 57198244-57199378 | chr7: 126891301-126894205 |
| chr2: 127643801-127644104 | chr3: 57994922-57995218 | chr7: 126987644-126988233 |
| chr2: 127729673-127729905 | chr3: 59035407-59035944 | chr7: 127880751-127881375 |
| chr2: 128173476-128173793 | chr3: 62304515-62304780 | chr7: 128509207-128510201 |
| chr2: 128180295-128181381 | chr3: 62859618-62861190 | chr7: 128530630-128531356 |
| chr2: 130691703-130692102 | chr3: 64430586-64431192 | chr7: 128809090-128809393 |
| chr2: 131129719-131130511 | chr3: 65534685-65535344 | chr7: 128828334-128829366 |
| chr2: 131513364-131514183 | chr3: 69129059-69129515 | chr7: 130126018-130126801 |
| chr2: 131720820-131721867 | chr3: 69434927-69435583 | chr7: 130417913-130419378 |
| chr2: 131792238-131793189 | chr3: 69591012-69592050 | chr7: 132260475-132262527 |
| chr2: 132121264-132121762 | chr3: 72788073-72788416 | chr7: 1329382-1329673 |
| chr2: 132285409-132285992 | chr3: 73673231-73674375 | chr7: 133812006-133812233 |
| chr2: 133173533-133174369 | chr3: 74663507-74664121 | chr7: 138666359-138666972 |
| chr2: 133426653-133428870 | chr3: 75834255-75834761 | chr7: 138720045-138721019 |
| chr2: 134023948-134024467 | chr3: 77088499-77089198 | chr7: 139167481-139168712 |
| chr2: 134785626-134785845 | chr3: 85008285-85008837 | chr7: 14029379-14029593 |
| chr2: 145273457-145275378 | chr3: 96532024-96533625 | chr7: 1408517-1408878 |
| chr2: 145281737-145282269 | chr3: 98451287-98452225 | chr7: 148036495-148036848 |
| chr2: 14772378-14775809 | chr3: 98620132-98620910 | chr7: 148982080-148982675 |
| chr2: 154334450-154335458 | chr3: 9904332-9904811 | chr7: 149119399-149120307 |
| chr2: 155554006-155556069 | chr3: 9956823-9957664 | chr7: 149495-150038 |
| chr2: 160654230-160654631 | chr3: 99594970-99595215 | chr7: 150019951-150020752 |
| chr2: 160918675-160919177 | chr3: 9993731-9994136 | chr7: 150655109-150655643 |
| chr2: 161263872-161264460 | chr4: 1005931-1006282 | chr7: 150675207-150675866 |
| chr2: 162930234-162930879 | chr4: 102711830-102712199 | chr7: 150676179-150676585 |
| chr2: 164592918-164593511 | chr4: 103997209-103998007 | chr7: 151078463-151079536 |
| chr2: 166649910-166650966 | chr4: 10462833-10463689 | chr7: 151136934-151137273 |
| chr2: 168149034-168150797 | chr4: 1049653-1049923 | chr7: 154861798-154862074 |
| chr2: 171569878-171573904 | chr4: 109683730-109684362 | chr7: 155579869-155580392 |
| chr2: 172944989-172945384 | chr4: 111532648-111533391 | chr7: 156810573-156814593 |
| chr2: 172945912-172946212 | chr4: 113435899-113438002 | chr7: 158785262-158785771 |
| chr2: 172947844-172948200 | chr4: 113445023-113445350 | chr7: 16460779-16461265 |
| chr2: 172949243-172950126 | chr4: 1161128-1161682 | chr7: 1703862-1710517 |
| chr2: 172951290-172952159 | chr4: 118006539-118006859 | chr7: 191876-193799 |
| chr2: 172952810-172953160 | chr4: 119273306-119274458 | chr7: 20817456-20818227 |
| chr2: 172959268-172962054 | chr4: 123747741-123748980 | chr7: 20823308-20825662 |
| chr2: 172971046-172971387 | chr4: 124333-124841 | chr7: 21582444-21583313 |
| chr2: 173600282-173600970 | chr4: 124426781-124427157 | chr7: 21985072-21985842 |
| chr2: 1746834-1748971 | chr4: 126235896-126238930 | chr7: 22893795-22894577 |
| chr2: 174877566-174877778 | chr4: 13529255-13529865 | chr7: 23513535-23514412 |
| chr2: 175193399-175193764 | chr4: 13548571-13549956 | chr7: 24323559-24325080 |
| chr2: 175199464-175202639 | chr4: 1396292-1401730 | chr7: 25891957-25892615 |
| chr2: 175204844-175207553 | chr4: 141294667-141295274 | chr7: 25898238-25898771 |
| chr2: 176989338-176989587 | chr4: 142053328-142054601 | chr7: 26191795-26192757 |
| chr2: 176993480-176995557 | chr4: 154143933-154144463 | chr7: 27190275-27191115 |
| chr2: 177001222-177001783 | chr4: 154170369-154170600 | chr7: 27194584-27194827 |
| chr2: 177005365-177005666 | chr4: 154605087-154606052 | chr7: 27208872-27209616 |
| chr2: 177029414-177029941 | chr4: 156588298-156589292 | chr7: 27212417-27214396 |
| chr2: 177052958-177054350 | chr4: 15704641-15705000 | chr7: 27219310-27219750 |
| chr2: 177502241-177502910 | chr4: 157892686-157893286 | chr7: 27224268-27224596 |
| chr2: 180725718-180726465 | chr4: 157997167-157997686 | chr7: 27275056-27275626 |
| chr2: 183902403-183903625 | chr4: 166299976-166300668 | chr7: 29603110-29603328 |
| chr2: 185463209-185463589 | chr4: 166794546-166795341 | chr7: 30028519-30029822 |
| chr2: 189156417-189157719 | chr4: 168155006-168155765 | chr7: 31232678-31232900 |
| chr2: 191044980-191045829 | chr4: 170946767-170947925 | chr7: 35301151-35301731 |
| chr2: 192109829-192110940 | chr4: 171010944-171011503 | chr7: 37487355-37488672 |
| chr2: 197457393-197458522 | chr4: 175750331-175750584 | chr7: 37960317-37961046 |
| chr2: 200320503-200329681 | chr4: 176922474-176922820 | chr7: 38670440-38671008 |
| chr2: 201450527-201451027 | chr4: 176923076-176923630 | chr7: 42276004-42277850 |
| chr2: 201983199-201983709 | chr4: 176986922-176987360 | chr7: 42533077-42533283 |
| chr2: 207308267-207308907 | chr4: 183369277-183370459 | chr7: 44079718-44080568 |
| chr2: 20865290-20867589 | chr4: 184319565-184320198 | chr7: 44364433-44365328 |
| chr2: 21022565-21022934 | chr4: 184643915-184644414 | chr7: 48128047-48128499 |
| chr2: 213402181-213403867 | chr4: 186455984-186456864 | chr7: 5111621-5112088 |
| chr2: 214148958-214149426 | chr4: 187476280-187476844 | chr7: 51383312-51384938 |
| chr2: 217236026-217237129 | chr4: 190942735-190944898 | chr7: 5336514-5336894 |
| chr2: 217497812-217498847 | chr4: 21950086-21950346 | chr7: 54731780-54732479 |
| chr2: 217556803-217557174 | chr4: 26030421-26030692 | chr7: 55000877-55001135 |
| chr2: 218621280-218621498 | chr4: 2765639-2766063 | chr7: 56183578-56184165 |
| chr2: 219156978-219157380 | chr4: 298804-299312 | chr7: 63767751-63767991 |
| chr2: 219252422-219252631 | chr4: 30718854-30719847 | chr7: 64349425-64350151 |
| chr2: 219748277-219748805 | chr4: 30721205-30724842 | chr7: 64407590-64408299 |
| chr2: 219762988-219763537 | chr4: 330163-332068 | chr7: 65037625-65037864 |
| chr2: 219773547-219774103 | chr4: 3464800-3465440 | chr7: 65509075-65509594 |
| chr2: 219827660-219828205 | chr4: 379900-380399 | chr7: 65878464-65878977 |
| chr2: 219866132-219868054 | chr4: 38869402-38869780 | chr7: 6654746-6655860 |
| chr2: 219922155-219923130 | chr4: 4108735-4109499 | chr7: 69062375-69065037 |
| chr2: 219924820-219926130 | chr4: 41218315-41218768 | chr7: 70060835-70061456 |
| chr2: 220159439-220159977 | chr4: 41646293-41646582 | chr7: 73021487-73021720 |
| chr2: 220196432-220196768 | chr4: 41753670-41753947 | chr7: 73867788-73868061 |
| chr2: 220348956-220349784 | chr4: 42399153-42400802 | chr7: 74202334-74203629 |
| chr2: 220361463-220363254 | chr4: 4577005-4577448 | chr7: 74572475-74573770 |
| chr2: 220377744-220377946 | chr4: 46391904-46392572 | chr7: 75889087-75889345 |
| chr2: 220406364-220406840 | chr4: 48271363-48271846 | chr7: 75889498-75889760 |
| chr2: 223288923-223290013 | chr4: 48272027-48272298 | chr7: 75896511-75896944 |
| chr2: 223536123-223536565 | chr4: 48492118-48493589 | chr7: 76026791-76027223 |
| chr2: 224701963-224702790 | chr4: 4854614-4855210 | chr7: 77649015-77649543 |
| chr2: 225906654-225907464 | chr4: 4873263-4873613 | chr7: 82072022-82073520 |
| chr2: 228582486-228582821 | chr4: 48908293-48908850 | chr7: 82791675-82792412 |
| chr2: 228736231-228736544 | chr4: 52917389-52918280 | chr7: 8473140-8475199 |
| chr2: 229045958-229046553 | chr4: 53617119-53617607 | chr7: 84814840-84816242 |
| chr2: 230135709-230136004 | chr4: 55991403-55992171 | chr7: 852961-853230 |
| chr2: 231692698-231693496 | chr4: 5713036-5713451 | chr7: 87256959-87258444 |
| chr2: 231712776-231712982 | chr4: 57687611-57687906 | chr7: 88388489-88389283 |
| chr2: 231902027-231903122 | chr4: 62065884-62068801 | chr7: 90225083-90226364 |
| chr2: 232276743-232277135 | chr4: 667274-667601 | chr7: 915754-916644 |
| chr2: 232765247-232765491 | chr4: 7194538-7195467 | chr7: 94536850-94537477 |
| chr2: 232791576-232792016 | chr4: 74486045-74486258 | chr7: 95025560-95026122 |
| chr2: 233367781-233368577 | chr4: 74702421-74702627 | chr7: 96631384-96631800 |
| chr2: 233740669-233741879 | chr4: 74809870-74810089 | chr7: 96746768-96747190 |
| chr2: 234776883-234777098 | chr4: 74864114-74864329 | chr7: 98971650-98973170 |
| chr2: 236578081-236580153 | chr4: 74964657-74965279 | chr7: 993402-994574 |
| chr2: 238395062-238396241 | chr4: 77507078-77507339 | chr7: 99768885-99769559 |
| chr2: 238599858-238601430 | chr4: 77610325-77610943 | chr8: 102092795-102093240 |
| chr2: 238768074-238768831 | chr4: 786119-786341 | chr8: 103822615-103823263 |
| chr2: 239755097-239758310 | chr4: 7940564-7941853 | chr8: 104310810-104311620 |
| chr2: 241391877-241393598 | chr4: 79472807-79473177 | chr8: 104383410-104384109 |
| chr2: 241395249-241395802 | chr4: 81123509-81124318 | chr8: 104510871-104513913 |
| chr2: 241771656-241772194 | chr4: 81124469-81124845 | chr8: 105235385-105235979 |
| chr2: 241855375-241855631 | chr4: 81951942-81952808 | chr8: 105600454-105601704 |
| chr2: 241922778-241923143 | chr4: 8594413-8594777 | chr8: 10586614-10586886 |
| chr2: 242498014-242499274 | chr4: 87515184-87515735 | chr8: 107669778-107670591 |
| chr2: 242742599-242743758 | chr4: 8869045-8870067 | chr8: 110374553-110374793 |
| chr2: 242785912-242786616 | chr4: 8893372-8896268 | chr8: 11058625-11058844 |
| chr2: 242808406-242808618 | chr4: 89378224-89378948 | chr8: 11059012-11059242 |
| chr2: 242823447-242823970 | chr4: 91048412-91049682 | chr8: 110986114-110986983 |
| chr2: 24300061-24300294 | chr4: 95678799-95679809 | chr8: 11555100-11555603 |
| chr2: 26396103-26397204 | chr5: 102201584-102201912 | chr8: 11759863-11760212 |
| chr2: 26401696-26402099 | chr5: 10307521-10307913 | chr8: 119963947-119964178 |
| chr2: 26407423-26408253 | chr5: 112630220-112630624 | chr8: 119964390-119964637 |
| chr2: 26726190-26726710 | chr5: 112823257-112824304 | chr8: 120220389-120221222 |
| chr2: 26785115-26785687 | chr5: 113391072-113392005 | chr8: 12990091-12990914 |
| chr2: 27341568-27341923 | chr5: 113696517-113699195 | chr8: 13133843-13134312 |
| chr2: 27958208-27958723 | chr5: 115151349-115152713 | chr8: 131455249-131456285 |
| chr2: 31360322-31361821 | chr5: 11903551-11904703 | chr8: 133492399-133493586 |
| chr2: 31456741-31457653 | chr5: 121412501-121414077 | chr8: 142318354-142319155 |
| chr2: 38763115-38763564 | chr5: 121647451-121648147 | chr8: 143530646-143530872 |
| chr2: 42068364-42068594 | chr5: 122424906-122425958 | chr8: 143531119-143534495 |
| chr2: 43864323-43864956 | chr5: 122426126-122426836 | chr8: 143556715-143556997 |
| chr2: 45161428-45163058 | chr5: 122433879-122435551 | chr8: 144099457-144099731 |
| chr2: 45235512-45237792 | chr5: 125930602-125931049 | chr8: 144503421-144503808 |
| chr2: 47499465-47499813 | chr5: 126564998-126565580 | chr8: 144640397-144640772 |
| chr2: 47796924-47799166 | chr5: 127872564-127874945 | chr8: 144798488-144799038 |
| chr2: 48757212-48757785 | chr5: 128300801-128301329 | chr8: 144822012-144822805 |
| chr2: 54785027-54785969 | chr5: 128795504-128797417 | chr8: 144842965-144843542 |
| chr2: 56410866-56412539 | chr5: 131346893-131347776 | chr8: 145555343-145562310 |
| chr2: 58273643-58274422 | chr5: 132155289-132155497 | chr8: 145697880-145698784 |
| chr2: 64836207-64836765 | chr5: 132946745-132948467 | chr8: 145700239-145700822 |
| chr2: 65086805-65087180 | chr5: 134824932-134825224 | chr8: 145806259-145806713 |
| chr2: 66660453-66660794 | chr5: 134827286-134827644 | chr8: 145909677-145912846 |
| chr2: 66661119-66661657 | chr5: 135170272-135171156 | chr8: 15397637-15398287 |
| chr2: 68870352-68871055 | chr5: 135527057-135528978 | chr8: 16884364-16885339 |
| chr2: 69240084-69241004 | chr5: 139017134-139017668 | chr8: 17354322-17355250 |
| chr2: 70351316-70351550 | chr5: 139040820-139041028 | chr8: 1771362-1772760 |
| chr2: 7057531-7058433 | chr5: 139047906-139048235 | chr8: 22089368-22089668 |
| chr2: 70994448-70995385 | chr5: 139076543-139077179 | chr8: 22408673-22409635 |
| chr2: 71192095-71192495 | chr5: 139081002-139081202 | chr8: 22456092-22456508 |
| chr2: 71503548-71504233 | chr5: 139135590-139135976 | chr8: 22457124-22457753 |
| chr2: 71680559-71681276 | chr5: 139174905-139176172 | chr8: 22960385-22960927 |
| chr2: 71693165-71694127 | chr5: 139227606-139228279 | chr8: 23081957-23082975 |
| chr2: 73151201-73152060 | chr5: 139742900-139743148 | chr8: 23260443-23261867 |
| chr2: 74347274-74347777 | chr5: 139927158-139927491 | chr8: 24770909-24772547 |
| chr2: 74425445-74426423 | chr5: 140011483-140012739 | chr8: 25897201-25897612 |
| chr2: 74725040-74727038 | chr5: 140167092-140168139 | chr8: 26371173-26372830 |
| chr2: 74776017-74776897 | chr5: 140207726-140208078 | chr8: 27183091-27183390 |
| chr2: 74781495-74782685 | chr5: 140261885-140262153 | chr8: 27348659-27348883 |
| chr2: 75147006-75147336 | chr5: 140531158-140532017 | chr8: 29210484-29210801 |
| chr2: 75426593-75428030 | chr5: 140568217-140569118 | chr8: 33371898-33372526 |
| chr2: 7571072-7571642 | chr5: 140573424-140574316 | chr8: 35092680-35093559 |
| chr2: 75787718-75788312 | chr5: 140604454-140605304 | chr8: 35093901-35094111 |
| chr2: 79220193-79220592 | chr5: 140615392-140616353 | chr8: 38325092-38326374 |
| chr2: 79739697-79740243 | chr5: 140620865-140621698 | chr8: 38644474-38645760 |
| chr2: 85359957-85362593 | chr5: 140741175-140741738 | chr8: 40755165-40755404 |
| chr2: 85640970-85641259 | chr5: 140750051-140750264 | chr8: 41624556-41625380 |
| chr2: 85811341-85811855 | chr5: 140762402-140762768 | chr8: 41654876-41655984 |
| chr2: 85980500-85982198 | chr5: 140767197-140767695 | chr8: 49231523-49231810 |
| chr2: 8818293-8818503 | chr5: 140777443-140777938 | chr8: 494156-496083 |
| chr2: 88316234-88316859 | chr5: 140802400-140802832 | chr8: 49647703-49647988 |
| chr2: 88469692-88470386 | chr5: 141132812-141133338 | chr8: 54569668-54570010 |
| chr2: 96054895-96055143 | chr5: 141228931-141230084 | chr8: 54789582-54790665 |
| chr2: 96192056-96193072 | chr5: 141705392-141705688 | chr8: 54791846-54795141 |
| chr2: 96314996-96315230 | chr5: 142782072-142785071 | chr8: 57232371-57232795 |
| chr2: 98703355-98703889 | chr5: 145316146-145316354 | chr8: 58907002-58907822 |
| chr20: 1206680-1207119 | chr5: 146257347-146258575 | chr8: 60030135-60032356 |
| chr20: 13200671-13202616 | chr5: 146614284-146614560 | chr8: 61193313-61194195 |
| chr20: 13975769-13976287 | chr5: 149681703-149682601 | chr8: 65491084-65491319 |
| chr20: 13976701-13977068 | chr5: 150004671-150004984 | chr8: 65492936-65494452 |
| chr20: 18039533-18039928 | chr5: 150284386-150284635 | chr8: 6691834-6693135 |
| chr20: 1874934-1875718 | chr5: 150325905-150326194 | chr8: 67089250-67089962 |
| chr20: 19738040-19739773 | chr5: 150537020-150537418 | chr8: 6949350-6950039 |
| chr20: 22562737-22566104 | chr5: 151304227-151304824 | chr8: 70946891-70947299 |
| chr20: 23028404-23032218 | chr5: 153853057-153853551 | chr8: 72917306-72917641 |
| chr20: 24898955-24899181 | chr5: 153862143-153862451 | chr8: 74005022-74005856 |
| chr20: 30196119-30196585 | chr5: 154026819-154027257 | chr8: 7543874-7544385 |
| chr20: 30582789-30583099 | chr5: 15500077-15500923 | chr8: 80695831-80696458 |
| chr20: 30605745-30605970 | chr5: 155107505-155108934 | chr8: 81805956-81806327 |
| chr20: 3229239-3229693 | chr5: 157002175-157003182 | chr8: 82192379-82193685 |
| chr20: 32450501-32450765 | chr5: 157285770-157286254 | chr8: 82644604-82644849 |
| chr20: 33865768-33866091 | chr5: 158758475-158758839 | chr8: 8820767-8821112 |
| chr20: 3387966-3388901 | chr5: 159343216-159343574 | chr8: 89339966-89340481 |
| chr20: 34188720-34190267 | chr5: 159738782-159739913 | chr8: 95651207-95651760 |
| chr20: 36793550-36793867 | chr5: 160974693-160975384 | chr8: 95652456-95652873 |
| chr20: 37230524-37230742 | chr5: 16179065-16180420 | chr8: 95653899-95654733 |
| chr20: 37274692-37275134 | chr5: 168727430-168728275 | chr8: 97156764-97158030 |
| chr20: 39311379-39312435 | chr5: 170288880-170289737 | chr8: 97505748-97507607 |
| chr20: 39316551-39319987 | chr5: 170877799-170878210 | chr8: 9756178-9756993 |
| chr20: 4202149-4202765 | chr5: 172068287-172069174 | chr8: 99305904-99306726 |
| chr20: 42285962-42286535 | chr5: 172385523-172385912 | chr9: 103173890-103174153 |
| chr20: 43438738-43439546 | chr5: 172710766-172711062 | chr9: 103790613-103791764 |
| chr20: 44098281-44099536 | chr5: 172754057-172757098 | chr9: 103791945-103792173 |
| chr20: 44452577-44453162 | chr5: 174151479-174152364 | chr9: 104248248-104249501 |
| chr20: 44539730-44540099 | chr5: 175223610-175224679 | chr9: 1051820-1052240 |
| chr20: 44642095-44642406 | chr5: 175621334-175621827 | chr9: 10612636-10613333 |
| chr20: 44746823-44747060 | chr5: 176236762-176238081 | chr9: 107509907-107510768 |
| chr20: 44935933-44937310 | chr5: 176789979-176790296 | chr9: 110249749-110252660 |
| chr20: 45142001-45142337 | chr5: 176830276-176831639 | chr9: 112262011-112262317 |
| chr20: 45523251-45524020 | chr5: 177098635-177099525 | chr9: 112402768-112403349 |
| chr20: 47443735-47445181 | chr5: 177366539-177366973 | chr9: 114287381-114287695 |
| chr20: 48598960-48599657 | chr5: 177433282-177434067 | chr9: 116111664-116112189 |
| chr20: 50158905-50159509 | chr5: 177540208-177541234 | chr9: 116450146-116450454 |
| chr20: 55500348-55501102 | chr5: 178016559-178017670 | chr9: 116860474-116860695 |
| chr20: 55839288-55839766 | chr5: 178322714-178323538 | chr9: 123631107-123631672 |
| chr20: 55840217-55841794 | chr5: 178367621-178368725 | chr9: 123690772-123691675 |
| chr20: 55964273-55964656 | chr5: 178770725-178772794 | chr9: 124061806-124062229 |
| chr20: 55964917-55965271 | chr5: 180479586-180480959 | chr9: 124461798-124462190 |
| chr20: 56323974-56324254 | chr5: 180542154-180542402 | chr9: 124498514-124498962 |
| chr20: 56725858-56726113 | chr5: 2038528-2038949 | chr9: 124975754-124976692 |
| chr20: 57224696-57226322 | chr5: 31855004-31855426 | chr9: 125109008-125109644 |
| chr20: 57581903-57582595 | chr5: 36690208-36690658 | chr9: 126135408-126136193 |
| chr20: 57797224-57797441 | chr5: 373843-374426 | chr9: 126762469-126762683 |
| chr20: 59826978-59828978 | chr5: 38556223-38557563 | chr9: 126807511-126808181 |
| chr20: 6103437-6103970 | chr5: 38845503-38846476 | chr9: 129677707-129678009 |
| chr20: 61147458-61147787 | chr5: 41510325-41510651 | chr9: 130461544-130461839 |
| chr20: 61200973-61201272 | chr5: 42423531-42423740 | chr9: 131012455-131013429 |
| chr20: 61456340-61456565 | chr5: 42424339-42425047 | chr9: 131965038-131965636 |
| chr20: 61884645-61886387 | chr5: 42994627-42994936 | chr9: 132020630-132021038 |
| chr20: 61927195-61927482 | chr5: 42995123-42995415 | chr9: 132082872-132083582 |
| chr20: 61937483-61937738 | chr5: 43017969-43018668 | chr9: 132099124-132099616 |
| chr20: 61992187-61993599 | chr5: 43040346-43040633 | chr9: 132145577-132146328 |
| chr20: 62600654-62601676 | chr5: 43040846-43041161 | chr9: 132331219-132331458 |
| chr20: 62673793-62674131 | chr5: 43396898-43397364 | chr9: 132359673-132360061 |
| chr20: 62714764-62715761 | chr5: 472601-474261 | chr9: 132382433-132383004 |
| chr20: 62958974-62959513 | chr5: 474959-475319 | chr9: 132499969-132500553 |
| chr20: 708602-709290 | chr5: 49736608-49737300 | chr9: 13278313-13279805 |
| chr20: 8112885-8113592 | chr5: 55776605-55777233 | chr9: 132934214-132934483 |
| chr20: 9048959-9050018 | chr5: 57878726-57879177 | chr9: 133308594-133309448 |
| chr20: 9819272-9819861 | chr5: 58334837-58335881 | chr9: 133412891-133413096 |
| chr21: 18984536-18985697 | chr5: 60921535-60922472 | chr9: 134151854-134153015 |
| chr21: 27011625-27012398 | chr5: 6448754-6449629 | chr9: 134158161-134158682 |
| chr21: 28216559-28218117 | chr5: 66299769-66300083 | chr9: 136451013-136451276 |
| chr21: 32929928-32932017 | chr5: 67584214-67584451 | chr9: 137217063-137218078 |
| chr21: 36041306-36043224 | chr5: 68710808-68711520 | chr9: 137299191-137299437 |
| chr21: 38119794-38120742 | chr5: 691081-691376 | chr9: 137533360-137534397 |
| chr21: 38352857-38353274 | chr5: 72415612-72416766 | chr9: 138985838-138987846 |
| chr21: 38362016-38362868 | chr5: 72715408-72715997 | chr9: 139014622-139014848 |
| chr21: 40032244-40033665 | chr5: 72732366-72733732 | chr9: 139159210-139159560 |
| chr21: 40760627-40760829 | chr5: 74349801-74350239 | chr9: 139551255-139551559 |
| chr21: 42878752-42880674 | chr5: 75378975-75380796 | chr9: 139552948-139553269 |
| chr21: 43373136-43374062 | chr5: 76011121-76012292 | chr9: 139553660-139553915 |
| chr21: 43917047-43917268 | chr5: 76115511-76116089 | chr9: 139595846-139596130 |
| chr21: 44073202-44074650 | chr5: 76941396-76941888 | chr9: 139872238-139873143 |
| chr21: 45148455-45149262 | chr5: 78365299-78365711 | chr9: 140051063-140051730 |
| chr21: 46129392-46129689 | chr5: 87437096-87437505 | chr9: 140317161-140318663 |
| chr21: 46351329-46352911 | chr5: 87976095-87976546 | chr9: 14348685-14349074 |
| chr21: 46706692-46707049 | chr5: 92906240-92908875 | chr9: 14349308-14349515 |
| chr22: 17849475-17850733 | chr5: 94619460-94621121 | chr9: 17134822-17135706 |
| chr22: 18923471-18923840 | chr5: 95170618-95170855 | chr9: 214587-215431 |
| chr22: 19753313-19755013 | chr5: 9544693-9546715 | chr9: 21559134-21559816 |
| chr22: 21319179-21319912 | chr5: 96038210-96038884 | chr9: 2241892-2242102 |
| chr22: 22862624-22863220 | chr6: 101841426-101841905 | chr9: 27528358-27528725 |
| chr9: 27528977-27529885 | | |
| chr9: 33044246-33044612 | | |
| chr9: 33447447-33447824 | | |
| chr9: 33750520-33751160 | | |
| chr9: 34377402-34377610 | | |
| chr9: 34379542-34380017 | | |
| chr9: 34577867-34578258 | | |
| chr9: 34589114-34591978 | | |
| chr9: 35756949-35757339 | | |
| chr9: 36036799-36037564 | | |
| chr9: 36258171-36258886 | | |
| chr9: 37575919-37576445 | | |
| chr9: 38069785-38069991 | | |
| chr9: 38423948-38424584 | | |
| chr9: 4297818-4300182 | | |
| chr9: 46148701-46149726 | | |
| chr9: 4662253-4662951 | | |
| chr9: 707022-707420 | | |
| chr9: 71788716-71789542 | | |
| chr9: 72658837-72659277 | | |
| chr9: 77502094-77502518 | | |
| chr9: 79073908-79074561 | | |
| chr9: 79520804-79521508 | | |
| chr9: 80911780-80912611 | | |
| chr9: 85677016-85678321 | | |
| chr9: 86571048-86572027 | | |
| chr9: 8857486-8858708 | | |
| chr9: 88713706-88714908 | | |
| chr9: 89560585-89562647 | | |
| chr9: 90112515-90113817 | | |
| chr9: 90340716-90341542 | | |
| chr9: 90589210-90589807 | | |
| chr9: 93563776-93564546 | | |
| chr9: 93955501-93956420 | | |
| chr9: 94183408-94183994 | | |
| chr9: 95569430-95572255 | | |
| chr9: 95896008-95897016 | | |
| chr9: 97021465-97021967 | | |
| chr9: 97766650-97767955 | | |
| chr9: 97810766-97811272 | | |
| chr9: 99145525-99145849 | | |

TABLE 3
Pan Cancer Methylation Sites

| | | |
|---|---|---|
| chr1: 10762450-10766925 | chr12: 101107864-101113622 | chr17: 48039283-48045064 |
| chr1: 110608266-110615303 | chr12: 103694091-103698418 | chr17: 48192635-48197085 |
| chr1: 113263574-113267787 | chr12: 104695349-104699984 | chr17: 48543571-48548900 |
| chr1: 113284333-113289172 | chr12: 106972413-106983086 | chr17: 4998370-5003205 |
| chr1: 114693137-114698672 | chr12: 113011100-113015529 | chr17: 50233176-50238466 |
| chr1: 115878168-115883332 | chr12: 113513165-113517970 | chr17: 59483574-59487780 |
| chr1: 116378360-116384364 | chr12: 113588807-113593304 | chr17: 59526980-59537254 |
| chr1: 1179757-1184470 | chr12: 113898751-113918717 | chr17: 6614423-6619471 |
| chr1: 119524783-119532712 | chr12: 114831912-114854360 | chr17: 6677206-6681710 |
| chr1: 119541057-119553320 | chr12: 114876144-114888579 | chr17: 70109980-70122442 |
| chr1: 12121489-12126148 | chr12: 115107504-115112061 | chr17: 71946479-71951255 |
| chr1: 145073484-145077845 | chr12: 117796077-117801448 | chr17: 72853622-72860012 |
| chr1: 146550329-146554577 | chr12: 119210111-119214393 | chr17: 72913569-72918510 |
| chr1: 1468605-1477220 | chr12: 120833587-120837927 | chr17: 73747619-73752178 |
| chr1: 147780067-147784473 | chr12: 122014171-122019693 | chr17: 74015770-74020658 |
| chr1: 149330994-149335389 | chr12: 123752050-123756373 | chr17: 74531282-74536566 |
| chr1: 155145186-155149444 | chr12: 127208779-127213651 | chr17: 75240872-75254180 |
| chr1: 155262319-155267536 | chr12: 127938452-127942907 | chr17: 75275318-75280172 |
| chr1: 155288607-155293001 | chr12: 129335871-129340653 | chr17: 75366689-75372506 |
| chr1: 156103708-156108171 | chr12: 130385610-130391139 | chr17: 75396285-75400527 |
| chr1: 156336759-156341251 | chr12: 130906778-130911191 | chr17: 75445478-75449821 |
| chr1: 156356051-156360252 | chr12: 131197825-131202157 | chr17: 77803867-77811046 |
| chr1: 156388404-156393581 | chr12: 132903450-132908206 | chr17: 7830533-7835164 |
| chr1: 156861416-156865711 | chr12: 14132627-14137242 | chr17: 78997641-79001641 |
| chr1: 160338605-160342843 | chr12: 15473319-15477901 | chr17: 7903928-7909445 |
| chr1: 161693638-161699298 | chr12: 184864-189610 | chr17: 79312963-79322653 |
| chr1: 164543541-164547917 | chr12: 29300035-29304954 | chr17: 79857809-79862963 |
| chr1: 165321704-165328328 | chr12: 3306813-3312270 | chr17: 932418-937088 |
| chr1: 16858874-16864296 | chr12: 3473011-3477654 | chr18: 11146308-11151936 |
| chr1: 170628457-170632851 | chr12: 41084523-41089102 | chr18: 11748954-11754756 |
| chr1: 173636663-173641045 | chr12: 45442203-45447386 | chr18: 12252148-12257089 |
| chr1: 175566377-175570808 | chr12: 48397169-48401372 | chr18: 13639585-13644415 |
| chr1: 177131393-177135846 | chr12: 49181050-49185282 | chr18: 13866533-13871026 |
| chr1: 179542721-179547307 | chr12: 49369691-49377550 | chr18: 19742937-19754363 |
| chr1: 180196120-180206975 | chr12: 49482921-49487178 | chr18: 30347691-30354302 |
| chr1: 181285301-181289873 | chr12: 5016586-5023171 | chr18: 35142908-35149628 |
| chr1: 181450707-181455073 | chr12: 5151013-5156346 | chr18: 43606141-43610510 |
| chr1: 18434552-18439673 | chr12: 52113411-52117679 | chr18: 44334184-44340100 |
| chr1: 18954896-18970739 | chr12: 52406382-52410675 | chr18: 44770993-44780084 |
| chr1: 19201875-19206234 | chr12: 52650019-52654743 | chr18: 44787407-44792678 |
| chr1: 197885089-197889791 | chr12: 53105913-53110471 | chr18: 54786960-54791194 |
| chr1: 200007808-200012036 | chr12: 53357193-53361507 | chr18: 55017708-55023605 |
| chr1: 201250453-201255648 | chr12: 53489573-53493955 | chr18: 55092826-55110853 |
| chr1: 202160959-202165390 | chr12: 54069054-54073265 | chr18: 55920988-55926068 |
| chr1: 202676882-202681769 | chr12: 54319302-54323721 | chr18: 56885092-56889665 |
| chr1: 203042723-203047390 | chr12: 54336762-54341168 | chr18: 56937625-56943540 |
| chr1: 208130328-208135117 | chr12: 54352530-54382102 | chr18: 58998684-59003692 |
| chr1: 214151215-214161080 | chr12: 54421428-54428709 | chr18: 61141927-61145927 |
| chr1: 21614381-21619101 | chr12: 54438643-54450091 | chr18: 70531966-70538871 |
| chr1: 217308750-217313178 | chr12: 54517769-54522457 | chr18: 72914108-72919233 |
| chr1: 221048449-221070185 | chr12: 57616770-57621402 | chr18: 73165403-73169920 |
| chr1: 225863069-225867328 | chr12: 58001881-58006249 | chr18: 74151240-74157073 |
| chr1: 226073151-226077680 | chr12: 58156856-58162000 | chr18: 74797145-74802038 |
| chr1: 226125113-226129695 | chr12: 63541637-63546967 | chr18: 74959557-74965822 |
| chr1: 228783987-228788204 | chr12: 6436273-6440931 | chr18: 76730971-76743244 |
| chr1: 231294560-231299345 | chr12: 65216246-65221143 | chr18: 77545966-77560948 |
| chr1: 24227116-24231537 | chr12: 65512879-65517863 | chr18: 902579-911574 |
| chr1: 243644395-243648888 | chr12: 72663684-72669551 | chr19: 10404935-10409342 |
| chr1: 248018331-248023252 | chr12: 75600992-75605344 | chr19: 10461627-10466378 |
| chr1: 25253528-25261005 | chr12: 81100035-81104716 | chr19: 1061545-1066265 |
| chr1: 2770127-2774665 | chr12: 81469570-81474119 | chr19: 1106395-1111610 |
| chr1: 29583898-29588598 | chr12: 99137387-99141769 | chr19: 11592373-11596987 |
| chr1: 2977276-2982758 | chr13: 100545634-100550911 | chr19: 12664244-12668682 |
| chr1: 32050472-32054771 | chr13: 100639335-100644188 | chr19: 12765750-12769980 |
| chr1: 34626784-34632976 | chr13: 102566426-102571495 | chr19: 12829794-12834225 |
| chr1: 34640383-34645024 | chr13: 108516335-108521063 | chr19: 12878575-12882888 |
| chr1: 36547555-36551965 | chr13: 109145799-109151019 | chr19: 13122960-13127259 |
| chr1: 38217703-38222012 | chr13: 112705805-112730419 | chr19: 13133318-13138169 |
| chr1: 38459585-38463988 | chr13: 112756599-112763113 | chr19: 13196700-13200999 |
| chr1: 38939920-38944404 | chr13: 20873519-20878214 | chr19: 13211451-13215821 |
| chr1: 39042060-39046561 | chr13: 27332227-27337205 | chr19: 13614753-13619267 |
| chr1: 39978366-39983768 | chr13: 28364550-28370505 | chr19: 14087571-14091796 |
| chr1: 40233768-40239190 | chr13: 28496227-28501046 | chr19: 15290400-15294632 |
| chr1: 40767187-40771871 | chr13: 28547840-28552246 | chr19: 1746168-1752243 |
| chr1: 41282848-41287149 | chr13: 32887117-32892116 | chr19: 18977352-18983200 |
| chr1: 41829977-41834542 | chr13: 36042845-36055119 | chr19: 19366709-19374393 |
| chr1: 44029287-44033853 | chr13: 51415372-51420149 | chr19: 21767190-21771786 |
| chr1: 46949169-46953792 | chr13: 53417898-53424872 | chr19: 2422006-2429983 |
| chr1: 47007576-47012132 | chr13: 58201587-58210930 | chr19: 30713550-30719970 |
| chr1: 4711990-4718555 | chr13: 79179945-79185880 | chr19: 33623468-33627805 |
| chr1: 47907713-47913020 | chr13: 84451665-84455897 | chr19: 35631410-35635697 |
| chr1: 50878917-50884103 | chr13: 93877246-93882877 | chr19: 36244329-36249982 |
| chr1: 50890438-50895243 | chr14: 101190852-101195499 | chr19: 36334276-36339138 |
| chr1: 53525573-53530974 | chr14: 101921576-101927995 | chr19: 36498170-36502530 |
| chr1: 53740298-53744845 | chr14: 103653242-103657928 | chr19: 36521392-36525887 |
| chr1: 55503061-55508015 | chr14: 105165664-105170129 | chr19: 3866587-3871217 |
| chr1: 61513876-61518831 | chr14: 24042887-24048760 | chr19: 38698334-38702577 |
| chr1: 63780395-63798140 | chr14: 24639054-24644220 | chr19: 38874071-38878332 |
| chr1: 65729412-65733849 | chr14: 24801679-24806353 | chr19: 39735690-39741288 |
| chr1: 65989002-65993811 | chr14: 29234836-29239832 | chr19: 39752974-39758540 |
| chr1: 66256441-66260918 | chr14: 29252366-29257069 | chr19: 40312927-40317144 |
| chr1: 67216080-67220293 | chr14: 33400095-33406079 | chr19: 405012-411511 |
| chr1: 67771330-67775767 | chr14: 36971170-36996488 | chr19: 42889312-42893646 |
| chr1: 77745315-77750224 | chr14: 37047334-37055690 | chr19: 44201559-44205987 |
| chr1: 86619279-86624871 | chr14: 37114189-37138348 | chr19: 44276274-44280777 |
| chr1: 91170103-91194804 | chr14: 38676246-38682937 | chr19: 45258353-45263809 |
| chr1: 91298980-91303891 | chr14: 38722255-38727537 | chr19: 45896880-45902315 |
| chr1: 92943908-92954609 | chr14: 48141434-48147589 | chr19: 45999831-46004686 |
| chr10: 100990157-100994687 | chr14: 51336713-51341146 | chr19: 46316491-46321266 |
| chr10: 101277942-101292338 | chr14: 52732208-52737486 | chr19: 46913312-46917802 |
| chr10: 102277163-102281730 | chr14: 54416678-54420881 | chr19: 47149769-47155125 |
| chr10: 102417148-102421668 | chr14: 57258879-57286558 | chr19: 48963003-48967792 |
| chr10: 102471207-102493011 | chr14: 58329677-58335121 | chr19: 49667276-49671552 |
| chr10: 102505483-102511646 | chr14: 60971773-60980180 | chr19: 50879419-50883664 |
| chr10: 102889011-102908693 | chr14: 61101979-61106663 | chr19: 50929271-50933638 |
| chr10: 102973970-102980096 | chr14: 62277477-62282019 | chr19: 51167660-51174023 |
| chr10: 102994035-102998646 | chr14: 69254677-69259036 | chr19: 51599823-51604260 |
| chr10: 103041991-103046480 | chr14: 74704189-74710192 | chr19: 51813158-51817458 |
| chr10: 105359785-105364188 | chr14: 77734734-77739772 | chr19: 54410711-54415087 |
| chr10: 105418686-105423076 | chr14: 85995469-86002478 | chr19: 54479413-54485572 |
| chr10: 105525044-105529044 | chr14: 92787495-92792712 | chr19: 55595978-55600887 |
| chr10: 106397568-106404812 | chr14: 95235623-95241679 | chr19: 55813941-55818277 |
| chr10: 108921781-108926805 | chr14: 95824676-95828941 | chr19: 56596039-56602296 |
| chr10: 109672197-109676964 | chr15: 100911439-100916022 | chr19: 56986314-56991741 |
| chr10: 110669725-110674326 | chr15: 23155795-23160624 | chr19: 58092740-58097764 |
| chr10: 111214605-111219083 | chr15: 27110031-27115479 | chr19: 5827049-5831474 |
| chr10: 118028733-118036230 | chr15: 27213952-27218856 | chr19: 58543116-58556587 |
| chr10: 118890162-118902329 | chr15: 33007531-33013696 | chr19: 7931264-7936898 |
| chr10: 118998436-119003530 | chr15: 33600817-33606003 | chr19: 8672333-8676764 |
| chr10: 119309205-119315563 | chr15: 35044444-35049480 | chr19: 868775-873318 |
| chr10: 119492494-119496991 | chr15: 37388176-37392380 | chr2: 102801673-102806556 |
| chr10: 120351693-120357821 | chr15: 40266582-40271061 | chr2: 105457128-105482760 |
| chr10: 121575530-121580385 | chr15: 45406468-45411528 | chr2: 106679983-106684403 |
| chr10: 123920851-123925542 | chr15: 47474370-47479499 | chr2: 107101834-107106053 |
| chr10: 124899908-124913035 | chr15: 49252985-49257564 | chr2: 108600825-108605467 |
| chr10: 125423496-125428642 | chr15: 53074188-53089488 | chr2: 114031360-114038041 |
| chr10: 125648821-125653373 | chr15: 53095562-53100476 | chr2: 114254776-114260043 |
| chr10: 125730221-125734843 | chr15: 59155046-59159594 | chr2: 118979770-118984466 |
| chr10: 129532411-129539366 | chr15: 60285108-60300520 | chr2: 119590603-119618826 |
| chr10: 130336696-130340994 | chr15: 67071307-67075943 | chr2: 119912127-119918663 |
| chr10: 130506444-130510658 | chr15: 74417871-74425044 | chr2: 124780253-124785255 |
| chr10: 131262948-131267947 | chr15: 76628030-76635515 | chr2: 127411697-127416171 |
| chr10: 134595358-134604649 | chr15: 79572831-79577211 | chr2: 127780614-127784829 |
| chr10: 15759424-15764101 | chr15: 79722100-79727643 | chr2: 128419720-128424182 |
| chr10: 16559605-16565822 | chr15: 89145661-89151198 | chr2: 130761484-130765764 |
| chr10: 1776785-1782018 | chr15: 89310720-89315183 | chr2: 132180328-132185101 |
| chr10: 22621351-22636862 | chr15: 89901447-89924768 | chr2: 137520461-137525696 |
| chr10: 22762709-22769050 | chr15: 89947374-89955182 | chr2: 139535693-139540650 |
| chr10: 23459301-23465889 | chr15: 91640909-91645702 | chr2: 142885725-142890553 |
| chr10: 23478698-23484455 | chr15: 96871409-96879721 | chr2: 144692667-144697180 |
| chr10: 23981367-23986978 | chr15: 96893307-96912030 | chr2: 154725907-154731328 |
| chr10: 26502384-26509434 | chr15: 96957342-96962531 | chr2: 157183558-157188355 |
| chr10: 27545669-27550402 | chr16: 10910160-10914719 | chr2: 162271295-162286677 |
| chr10: 43426168-43431460 | chr16: 20082708-20087305 | chr2: 171669599-171682358 |
| chr10: 48436412-48441320 | chr16: 2226191-2232946 | chr2: 176929576-176984402 |
| chr10: 50600990-50608783 | chr16: 22822617-22828459 | chr2: 177010372-177027692 |
| chr10: 50815602-50822356 | chr16: 23722271-23726775 | chr2: 177034255-177045444 |
| chr10: 63210496-63215009 | chr16: 24265041-24269527 | chr2: 182319762-182325029 |
| chr10: 71329450-71335392 | chr16: 3011017-3015228 | chr2: 182519222-182523927 |
| chr10: 75405414-75409706 | chr16: 3065522-3070358 | chr2: 19558964-19563650 |
| chr10: 76571196-76575507 | chr16: 31051480-31055800 | chr2: 198027069-198031438 |
| chr10: 8074003-8080378 | chr16: 3188766-3193389 | chr2: 200331688-200336172 |
| chr10: 88120925-88129364 | chr16: 3218439-3223356 | chr2: 207504775-207509422 |
| chr10: 94178316-94182754 | chr16: 48842552-48847264 | chr2: 20868007-20873280 |
| chr10: 94453525-94457896 | chr16: 49307124-49319263 | chr2: 219734133-219738788 |
| chr10: 94818027-94831040 | chr16: 49870450-49874926 | chr2: 219846292-219860917 |
| chr10: 99787615-99793320 | chr16: 51145491-51149944 | chr2: 220171871-220176283 |
| chr11: 105479127-105483422 | chr16: 51166267-51171110 | chr2: 220297484-220302243 |
| chr11: 115628399-115633117 | chr16: 51181700-51190763 | chr2: 220410342-220414678 |
| chr11: 119291321-119295943 | chr16: 54323041-54327703 | chr2: 223157726-223187468 |
| chr11: 123064518-123068986 | chr16: 54968302-54974846 | chr2: 233249362-233255414 |
| chr11: 124627724-124631926 | chr16: 55362824-55367483 | chr2: 237066072-237080762 |
| chr11: 128417199-128421513 | chr16: 55511221-55515526 | chr2: 238862316-238867170 |
| chr11: 128692085-128696688 | chr16: 58028215-58033633 | chr2: 241457633-241462047 |
| chr11: 131778329-131783532 | chr16: 6066915-6072401 | chr2: 241756142-241762783 |
| chr11: 132811563-132816395 | chr16: 62067122-62072634 | chr2: 25497764-25502429 |
| chr11: 132932060-132936291 | chr16: 66610750-66615412 | chr2: 30451567-30457655 |
| chr11: 132950539-132955307 | chr16: 67206068-67210678 | chr2: 31803294-31808403 |
| chr11: 133992710-133997090 | chr16: 67569253-67574728 | chr2: 3284325-3288530 |
| chr11: 14993129-14997908 | chr16: 67916680-67920909 | chr2: 3748829-3753927 |
| chr11: 17738790-17745779 | chr16: 68478865-68484822 | chr2: 38299277-38306518 |
| chr11: 20179201-20184325 | chr16: 71457782-71462338 | chr2: 45153196-45159049 |
| chr11: 20616198-20625399 | chr16: 82658652-82663813 | chr2: 45167506-45173884 |
| chr11: 27741473-27746564 | chr16: 84000270-84004860 | chr2: 45225645-45230783 |
| chr11: 2888389-2893337 | chr16: 86528748-86534994 | chr2: 45238373-45243579 |
| chr11: 31823744-31850776 | chr16: 86547070-86552512 | chr2: 45393870-45400186 |
| chr11: 32450145-32459311 | chr16: 86609389-86615821 | chr2: 465850-470659 |
| chr11: 36395927-36401398 | chr16: 88941428-88945669 | chr2: 50572046-50576817 |
| chr11: 43566922-43571854 | chr17: 1171536-1176733 | chr2: 54084777-54089266 |
| chr11: 44323658-44329932 | chr17: 12566668-12571335 | chr2: 5829188-5834208 |
| chr11: 46297545-46302216 | chr17: 12875271-12879773 | chr2: 63280515-63289097 |
| chr11: 46364877-46369101 | chr17: 14199727-14204052 | chr2: 66650692-66656218 |
| chr11: 60716429-60720888 | chr17: 14246392-14250721 | chr2: 66670432-66675636 |
| chr11: 61535001-61539001 | chr17: 15818621-15823325 | chr2: 66806569-66811404 |
| chr11: 624729-642628 | chr17: 1878790-1883116 | chr2: 71785431-71789897 |
| chr11: 64134815-64140187 | chr17: 19881326-19885610 | chr2: 73141056-73150260 |
| chr11: 64476844-64481598 | chr17: 21365115-21369592 | chr2: 80527678-80532846 |
| chr11: 64813041-64817722 | chr17: 27897512-27902067 | chr2: 80547579-80551798 |
| chr11: 65350232-65355134 | chr17: 32482008-32486280 | chr2: 87013975-87020182 |
| chr11: 65407637-65412127 | chr17: 33774554-33778888 | chr2: 87086817-87091037 |
| chr11: 65814405-65818665 | chr17: 35289900-35302875 | chr2: 97190978-97195383 |
| chr11: 67348566-67353565 | chr17: 36715692-36720593 | chr20: 10196136-10200984 |
| chr11: 68620109-68624339 | chr17: 37319483-37324099 | chr20: 17204529-17210756 |
| chr11: 69515841-69521929 | chr17: 37761692-37766304 | chr20: 21374359-21380245 |
| chr11: 69829572-69834484 | chr17: 40935259-40939480 | chr20: 21483933-21498714 |
| chr11: 70209532-70213532 | chr17: 41275001-41280000 | chr20: 21684200-21697344 |
| chr11: 70506329-70510617 | chr17: 43035167-43039740 | chr20: 22546968-22561240 |
| chr11: 70670835-70675055 | chr17: 43470528-43476343 | chr20: 25061839-25067525 |
| chr11: 71950113-71954528 | chr17: 45947677-45951885 | chr20: 2537134-2541877 |
| chr11: 723597-728870 | chr17: 46602363-46610390 | chr20: 2727998-2733630 |
| chr11: 72530613-72535774 | chr17: 46618368-46634212 | chr20: 2778979-2783497 |
| chr11: 79146359-79154200 | chr17: 46667435-46676181 | chr20: 3143122-3147746 |
| chr11: 8188227-8192671 | chr17: 46689521-46699701 | chr20: 32854660-32859248 |
| chr11: 88239711-88244562 | chr17: 46794235-46802746 | chr20: 33294515-33300242 |
| chr11: 89222417-89226718 | chr17: 46822786-46827372 | chr20: 36010596-36015439 |
| chr20: 36224618-36228841 | chr6: 161186085-161190639 | chr20: 44655464-44661243 |
| chr20: 37350131-37359372 | chr6: 1617094-1623094 | |
| chr20: 39992546-39997810 | chr6: 166577974-166585423 | |
| chr20: 41815476-41821212 | chr6: 166664838-166669541 | |
| chr20: 44683772-44689610 | chr6: 170730120-170734442 | |
| chr20: 48182194-48186833 | chr6: 26612014-26616851 | |
| chr20: 51587708-51592020 | chr6: 27226101-27230364 | |
| chr20: 52787253-52792986 | chr6: 29593299-29597795 | |
| chr20: 5294267-5299798 | chr6: 29892141-29897117 | |
| chr20: 57087461-57092237 | chr6: 30093174-30097610 | |
| chr20: 57413136-57429047 | chr6: 30137719-30142263 | |
| chr20: 61701527-61706022 | chr6: 33046417-33050814 | |
| chr20: 688576-693099 | chr6: 33391593-33395908 | |
| chr20: 9494472-9498893 | chr6: 33653967-33658238 | |
| chr21: 19615099-19619874 | chr6: 35477389-35481678 | |
| chr21: 31309387-31314106 | chr6: 37614723-37619179 | |
| chr21: 32622145-32626382 | chr6: 38680950-38685265 | |
| chr21: 34393129-34402245 | chr6: 389189-395790 | |
| chr21: 38063180-38068185 | chr6: 4077053-4081443 | |
| chr21: 38074763-38083833 | chr6: 41526267-41530900 | |
| chr21: 42216490-42221222 | chr6: 41906746-41911711 | |
| chr22: 19744156-19748369 | chr6: 42070033-42074701 | |
| chr22: 19965280-19969808 | chr6: 42143848-42148053 | |
| chr22: 25079851-25084112 | chr6: 42877280-42881623 | |
| chr22: 29707282-29714013 | chr6: 46653263-46658738 | |
| chr22: 31196492-31201033 | chr6: 50680335-50685214 | |
| chr22: 31498397-31503239 | chr6: 50785287-50793573 | |
| chr22: 37210770-37215467 | chr6: 50808643-50820431 | |
| chr22: 37463057-37467331 | chr6: 55037171-55041392 | |
| chr22: 37909980-37914258 | chr6: 5995028-6009797 | |
| chr22: 38377094-38381964 | chr6: 70990041-70994912 | |
| chr22: 38474837-38480839 | chr6: 7227878-7232865 | |
| chr22: 39260339-39265211 | chr6: 72296275-72300528 | |
| chr22: 42303618-42309254 | chr6: 78170232-78176088 | |
| chr22: 42320044-42324909 | chr6: 85470703-85476132 | |
| chr22: 42683895-42688095 | chr6: 99273764-99278038 | |
| chr22: 44255943-44260612 | chr6: 99288280-99292771 | |
| chr22: 44285498-44290061 | chr7: 100073304-100077551 | |
| chr22: 44724725-44729590 | chr7: 100815485-100825701 | |
| chr22: 46316694-46321087 | chr7: 101003900-101009443 | |
| chr22: 46438394-46443019 | chr7: 103083711-103088132 | |
| chr22: 48882885-48889043 | chr7: 103966784-103971959 | |
| chr22: 50494442-50499393 | chr7: 113722925-113729795 | |
| chr3: 11032447-11037384 | chr7: 12149221-12153559 | |
| chr3: 113158300-113162641 | chr7: 121938007-121959341 | |
| chr3: 121900743-121905645 | chr7: 124402175-124406432 | |
| chr3: 126111548-126115967 | chr7: 127988927-127994616 | |
| chr3: 127631994-127636588 | chr7: 128553330-128558650 | |
| chr3: 127792370-127798136 | chr7: 129418287-129425355 | |
| chr3: 12836472-12840782 | chr7: 130788359-130794773 | |
| chr3: 128717866-128723245 | chr7: 1360812-1365643 | |
| chr3: 129691128-129696841 | chr7: 136551855-136558194 | |
| chr3: 13112628-13117245 | chr7: 142492564-142497248 | |
| chr3: 133391119-133395657 | chr7: 143580126-143584610 | |
| chr3: 137480965-137493004 | chr7: 149387655-149391976 | |
| chr3: 138654628-138661107 | chr7: 149742403-149748469 | |
| chr3: 147106512-147116479 | chr7: 152619917-152624149 | |
| chr3: 147124989-147144391 | chr7: 153746408-153752444 | |
| chr3: 154144348-154148965 | chr7: 153999965-154004281 | |
| chr3: 157810054-157823836 | chr7: 155162558-155177248 | |
| chr3: 170301045-170305768 | chr7: 155239324-155245757 | |
| chr3: 172163373-172168738 | chr7: 155256828-155263403 | |
| chr3: 184054420-184058671 | chr7: 155300254-155305158 | |
| chr3: 185909345-185914228 | chr7: 155593693-155607095 | |
| chr3: 186076711-186082111 | chr7: 156407024-156411865 | |
| chr3: 19187689-19192100 | chr7: 156793356-156803632 | |
| chr3: 192123822-192129994 | chr7: 156869055-156873297 | |
| chr3: 22411493-22416365 | chr7: 158934508-158940492 | |
| chr3: 236392-242140 | chr7: 19143873-19148256 | |
| chr3: 26662105-26666796 | chr7: 19182819-19187033 | |
| chr3: 27769639-27773942 | chr7: 20368004-20373504 | |
| chr3: 32859142-32863429 | chr7: 20828568-20832817 | |
| chr3: 3838514-3844772 | chr7: 23285222-23289508 | |
| chr3: 44061315-44065837 | chr7: 26413747-26418891 | |
| chr3: 44594536-44599018 | chr7: 27132098-27136736 | |
| chr3: 46616308-46620669 | chr7: 27144070-27150389 | |
| chr3: 49945622-49950430 | chr7: 27180614-27187562 | |
| chr3: 55506337-55510708 | chr7: 27195602-27208462 | |
| chr3: 62352292-62365082 | chr7: 27225521-27231043 | |
| chr3: 63261990-63266205 | chr7: 27258102-27262467 | |
| chr3: 64251534-64255819 | chr7: 27276946-27294197 | |
| chr3: 6900824-6906641 | chr7: 30719373-30724445 | |
| chr3: 71832069-71836653 | chr7: 32108064-32112910 | |
| chr3: 75665778-75671067 | chr7: 35294922-35300218 | |
| chr3: 75953760-75958308 | chr7: 37953623-37958555 | |
| chr3: 87839797-87844563 | chr7: 42265547-42269823 | |
| chr3: 9175692-9180189 | chr7: 43150021-43155340 | |
| chr4: 100868378-100873994 | chr7: 49811009-49817752 | |
| chr4: 105147-109898 | chr7: 53284852-53289192 | |
| chr4: 107954556-107959453 | chr7: 54610325-54614558 | |
| chr4: 109091039-109096546 | chr7: 56353509-56357798 | |
| chr4: 110220971-110226257 | chr7: 6588564-6592957 | |
| chr4: 111552966-111557504 | chr7: 6659876-6664695 | |
| chr4: 114898356-114902810 | chr7: 70594229-70600382 | |
| chr4: 122299568-122304290 | chr7: 71798758-71804768 | |
| chr4: 128542032-128546903 | chr7: 72836384-72840815 | |
| chr4: 134067163-134072442 | chr7: 73892816-73897110 | |
| chr4: 13522063-13528083 | chr7: 749713-754150 | |
| chr4: 140199065-140203449 | chr7: 87561343-87566571 | |
| chr4: 144618823-144624218 | chr7: 89745893-89751036 | |
| chr4: 147557206-147563901 | chr7: 90891568-90898683 | |
| chr4: 151502012-151507085 | chr7: 95223504-95228194 | |
| chr4: 154707513-154716240 | chr7: 96648222-96654246 | |
| chr4: 155661810-155666315 | chr7: 97359133-97365018 | |
| chr4: 156127169-156132209 | chr7: 97839637-97844005 | |
| chr4: 156678096-156683386 | chr8: 101115923-101120693 | |
| chr4: 15777999-15782729 | chr8: 102502479-102506841 | |
| chr4: 158141297-158146053 | chr8: 105476673-105481340 | |
| chr4: 164262822-164267772 | chr8: 11534768-11540961 | |
| chr4: 169797087-169801625 | chr8: 11555853-11569212 | |
| chr4: 172731735-172737118 | chr8: 120426399-120431178 | |
| chr4: 174420025-174462054 | chr8: 130993922-130998149 | |
| chr4: 185935243-185944747 | chr8: 132050204-132056749 | |
| chr4: 187217321-187221745 | chr8: 139506796-139511774 | |
| chr4: 188914606-188918876 | chr8: 142526186-142531029 | |
| chr4: 190935926-190942591 | chr8: 143543446-143548178 | |
| chr4: 204378-208892 | chr8: 144806222-144812978 | |
| chr4: 24799110-24803902 | chr8: 144988271-145004135 | |
| chr4: 25088107-25092510 | chr8: 145101286-145110027 | |
| chr4: 41747185-41751811 | chr8: 145923411-145928101 | |
| chr4: 41867175-41884964 | chr8: 21642909-21649845 | |
| chr4: 46993129-46997872 | chr8: 21903462-21907757 | |
| chr4: 47032428-47036940 | chr8: 23560476-23569678 | |
| chr4: 4857633-4871173 | chr8: 24810947-24816299 | |
| chr4: 54964164-54978202 | chr8: 25898563-25907842 | |
| chr4: 5707986-5712495 | chr8: 26719643-26726566 | |
| chr4: 57519622-57524703 | chr8: 37820487-37826008 | |
| chr4: 5889204-5897116 | chr8: 41422342-41427300 | |
| chr4: 66533194-66537620 | chr8: 4846969-4854635 | |
| chr4: 680725-685079 | chr8: 49466684-49470959 | |
| chr4: 81107888-81121391 | chr8: 50820271-50824860 | |
| chr4: 85401831-85425190 | chr8: 53849702-53856426 | |
| chr4: 90226715-90231010 | chr8: 55364181-55382186 | |
| chr4: 93224349-93229007 | chr8: 57356127-57361415 | |
| chr4: 94753787-94758310 | chr8: 65279904-65292946 | |
| chr4: 959348-964155 | chr8: 65708991-65713722 | |
| chr5: 11382682-11387521 | chr8: 68862585-68866946 | |
| chr5: 115695135-115699589 | chr8: 70979874-70986888 | |
| chr5: 122428677-122433443 | chr8: 72466561-72471561 | |
| chr5: 134361093-134388370 | chr8: 85094760-85099247 | |
| chr5: 139136876-139141242 | chr8: 86348766-86353196 | |
| chr5: 140050060-140055381 | chr8: 87079654-87084046 | |
| chr5: 140303713-140309193 | chr8: 97167732-97174022 | |
| chr5: 140344106-140348931 | chr8: 9758751-9766748 | |
| chr5: 140785448-140790044 | chr8: 98287605-98292404 | |
| chr5: 140796758-140801359 | chr8: 99958498-99963438 | |
| chr5: 140808495-140814617 | chr8: 99982585-99988983 | |
| chr5: 140862528-140866748 | chr9: 100608697-100622192 | |
| chr5: 145716290-145727852 | chr9: 102588743-102593303 | |
| chr5: 146886751-146891840 | chr9: 104497850-104503076 | |
| chr5: 148031473-148036080 | chr9: 112079403-112084905 | |
| chr5: 158476379-158480630 | chr9: 115820072-115825416 | |
| chr5: 158521907-158526598 | chr9: 120173254-120179496 | |
| chr5: 159397005-159401928 | chr9: 120505228-120509642 | |
| chr5: 170733170-170746107 | chr9: 122129087-122134214 | |
| chr5: 172108283-172113166 | chr9: 123654751-123658972 | |
| chr5: 172657050-172674971 | chr9: 124411513-124416193 | |
| chr5: 174156681-174161729 | chr9: 124985744-124993086 | |
| chr5: 175083005-175087756 | chr9: 126771247-126782953 | |
| chr5: 178419226-178424337 | chr9: 129370738-129391231 | |
| chr5: 179226284-179231003 | chr9: 131152347-131157923 | |
| chr5: 180484155-180488892 | chr9: 132457588-132462017 | |
| chr5: 1872908-1889743 | chr9: 133532535-133544394 | |
| chr5: 2736954-2759024 | chr9: 134427867-134432491 | |
| chr5: 31191953-31196419 | chr9: 135036714-135041978 | |
| chr5: 3588645-3605054 | chr9: 135453165-135468240 | |
| chr5: 37832672-37837128 | chr9: 136292739-136297236 | |
| chr5: 38255826-38261136 | chr9: 137965111-137969727 | |
| chr5: 45693395-45698510 | chr9: 139094666-139098993 | |
| chr5: 50683454-50688148 | chr9: 139394206-139399040 | |
| chr5: 52775789-52779996 | chr9: 139713664-139718441 | |
| chr5: 54517055-54529760 | chr9: 16724860-16729273 | |
| chr5: 59187047-59191894 | chr9: 17904420-17909488 | |
| chr5: 63253045-63259886 | chr9: 19786216-19791288 | |
| chr5: 71012918-71017715 | chr9: 21968914-21973190 | |
| chr5: 72524204-72531976 | chr9: 22003888-22008229 | |
| chr5: 72592148-72597808 | chr9: 23818692-23824135 | |
| chr5: 72674121-72680421 | chr9: 23848911-23853522 | |
| chr5: 76921888-76938984 | chr9: 32780937-32785625 | |
| chr5: 77138543-77149785 | chr9: 35842850-35846850 | |
| chr5: 77251833-77256049 | chr9: 36737535-36741782 | |
| chr5: 77266351-77270787 | chr9: 37000490-37004957 | |
| chr5: 77803754-77808313 | chr9: 77110713-77115927 | |
| chr5: 87966636-87972070 | chr9: 79631327-79640169 | |
| chr5: 87978879-87987810 | chr9: 86150354-86155777 | |
| chr5: 88183225-88187589 | chr9: 91790663-91795611 | |
| chr5: 92921488-92926497 | chr9: 95475297-95479708 | |
| chr5: 92937796-92942216 | chr9: 96106467-96110992 | |
| chr6: 100036542-100041477 | chr9: 96708812-96720186 | |
| chr6: 100895008-100917245 | chr9: 967530-975276 | |
| chr6: 101844767-101849135 | chr9: 97399287-97404067 | |
| chr6: 10379559-10392565 | chr9: 98109365-98114362 | |
| chr6: 106427112-106436459 | chrX: 152610776-152615464 | |
| chr6: 108483672-108499996 | chrX: 67350651-67354923 | |
| chr6: 10879847-10884051 | chrX: 99889300-99893794 | |
| chr6: 110297366-110303267 | | |
| chr6: 117196090-117200705 | | |
| chr6: 117589534-117594279 | | |
| chr6: 117867098-117871530 | | |
| chr6: 127439554-127443760 | | |
| chr6: 134208640-134213218 | | |
| chr6: 134636798-134641021 | | |
| chr6: 137240316-137247442 | | |
| chr6: 1376446-1396170 | | |
| chr6: 137807343-137819223 | | |
| chr6: 138743349-138747593 | | |
| chr6: 150333526-150338278 | | |
| chr6: 150356873-150361394 | | |
| chr6: 154358587-154363008 | | |
| chr6: 168839439-168843699 | | |

What is claimed is:

1. A method of characterizing a cell-free DNA (cfDNA) sample from a subject, comprising:
a) subjecting the cfDNA sample to whole genome bisulfite sequencing, reduced representation bisulfite sequencing or targeted bisulfite sequencing to generate sequencing data comprising reads of methylation sequences for a genomic sequence from the cfDNA sample, wherein the genomic sequence comprises one or more CpG Islands (CGIs) selected from Table 2 or Table 3 that are methylated in the genome of extraembryonic ectoderm (ExE) and not methylated in corresponding epiblast or adult tissue;
b) analyzing the sequencing data comprising reads of methylation sequences for the genomic sequence comprising the one or more CGIs selected from Table 2 or Table 3; and
c) classifying the cfDNA sample using the analyzed sequence data.

2. The method of claim 1, wherein the sequencing data comprises sequence information for less than 0.3% of the genome of the subject.

3. The method of claim 1, wherein the sequencing data comprises sequence information substantially limited to one or more regions of the subject's genome having a plurality of CGI methylated in the genome of ExE and not methylated in corresponding epiblast or adult tissue.

4. The method of claim 1, wherein the sequencing data comprising reads of methylation sequences for a genomic sequence from the cfDNA sample has been enriched for sequences comprising methylation.

5. The method of claim 4, wherein the enrichment comprises an MBD2 protein-based enrichment method.

6. The method of claim 1, wherein the genomic sequence comprises a contiguous sequence of about 8 megabases of the human genome comprising a plurality of CpG Islands (CGIs) methylated in the genome of extraembryonic ectoderm (ExE) and/or one or more regions identified in Table 3; or
wherein the genomic sequence comprises 50-75 CGIs methylated in the genome of ExE.

7. The method of claim 1, wherein the cfDNA is classified as originating from a tumor.

8. The method of claim 1, wherein the cfDNA is classified as originating from one or more tumor types or tissues.

9. The method of claim 8, wherein the one or more tumor types comprise one or more of acute myeloid leukemia, bladder cancer, breast cancer, colon cancer, esophageal cancer, kidney cancer, liver cancer, lung cancer, ovarian cancer, pancreatic cancer, prostate cancer, or stomach cancer.

10. The method of claim 1, wherein the cfDNA is classified as comprising fully methylated reads.

11. A method of characterizing a cell-free DNA (cfDNA) sample from a subject, comprising
a) subjecting a cfDNA sample from the subject to whole genome bisulfite sequencing, reduced representation bisulfite sequencing or targeted bisulfite sequencing to generate sequencing data comprising reads of methylation sequences for a genomic sequence from the cfDNA sample, wherein the genomic sequence comprises a plurality of CpG Islands (CGI) methylated in the genome of extraembryonic ectoderm (ExE) and not methylated in corresponding epiblast or adult tissue;
b) receiving the sequencing data comprising reads of methylation sequences for the genomic sequence from the cfDNA sample;
c) applying a machine learning method to estimate a probability distribution of haplotypes across a plurality of CGIs in the genome of ExE; and
d) determining a classification of tumor versus normal based on a prediction score obtained from the machine learning method.

12. The method of claim 11, wherein the machine learning method is random forest, deep learning, or is a support vector machine.

13. A method of characterizing a cell-free DNA (cfDNA) sample from a subject, comprising
a) subjecting a cfDNA sample obtained from the subject after a cancer treatment to whole genome bisulfite sequencing, reduced representation bisulfite sequencing or targeted bisulfite sequencing to generate sequencing data comprising reads of methylation sequences for a genomic sequence from the cfDNA sample;
b) receiving the sequencing data comprising reads of methylation sequences for the genomic sequence from the cfDNA sample;
c) determining a quantity of haplotypes comprising five methylated CpG sites; and
d) characterizing the cfDNA sample as comprising fully methylated cfDNA if the quantity of the haplotypes is greater than a significance threshold.

14. The method of claim 13, further comprising detecting cancer in the subject when the cfDNA sample is characterized as comprising fully methylated cfDNA, or detecting eradication of cancer in the subject when the cfDNA sample is characterized as not comprising fully methylated cfDNA.

15. The method of claim 13, wherein the sequencing data comprises sequence information for less than 0.3% of the genome of the subject.

16. The method of claim 13, wherein the sequencing data comprises sequence information substantially limited to one or more regions of the subject's genome having a plurality of CGI methylated in the genome of ExE and not methylated in corresponding epiblast or adult tissue.

17. The method of claim 13, wherein the sequencing data comprising reads of methylation sequences for a genomic sequence from the cfDNA sample has been enriched for sequences comprising methylation, optionally wherein the enrichment comprises an MBD2 protein-based enrichment method.

18. The method of claim 13, wherein the five CpG sites are five consecutive CpG sites.

19. The method of claim 13, wherein the genomic sequence comprises a contiguous sequence of about 8 megabases of the human genome comprising a plurality of CpG Islands (CGIs) methylated in the genome of extraembryonic ectoderm (ExE) and/or one or more regions identified in Table 3; or
wherein the genomic sequence comprises 50-75 CGIs methylated in the genome of ExE.

20. The method of claim 13, further comprising a step of determining a tissue of origin from the sequencing data.
